(19) United States
(12) Patent Application Publication
(10) Pub. No.: US 2020/0149042 A1
(43) Pub. Date: May 14, 2020

(54) MODULATING BIOMARKERS TO INCREASE TUMOR IMMUNITY AND IMPROVE THE EFFICACY OF CANCER IMMUNOTHERAPY

(71) Applicants: William N. HAINING, Newton, MA (US); Robert MANGUSO, Boston, MA (US); Dana-Farber Cancer Institute, Inc., Boston, MA (US)

(72) Inventors: William N. Haining, Newton, MA (US); Robert Manguso, Boston, MA (US)

(21) Appl. No.: 16/629,169

(22) PCT Filed: Jul. 16, 2018
(86) PCT No.: PCT/US18/42242

§ 371 Date: Jan. 7, 2020

Related U.S. Application Data
(60) Provisional application No. 62/532,589, filed on Jul. 14, 2017.

(51) Int. Cl. C12N 15/113; A01K 67/027; A61P 35/00

(52) U.S. Cl. CPC C12N 15/113; A01K 67/0278; A61P 35/00; C12N 2310/20; C12N 2310/141; A01K 2207/15; A01K 2207/05; A01K 2227/105; C07K 2317/56; C07K 2317/24

(57) ABSTRACT

The present invention relates, in part, to methods of treating a cancer in a subject comprising administering to the subject a therapeutically effective amount of an agent that inhibits one or more biomarkers listed in Table 1, such as regulators of ubiquitin proteasome pathway (e.g., STIP1 homology and U-Box containing protein 1 (STUB 1), ubiquilin-1 (UBQLN1), and heat shock protein 90 kDa beta member 1 (HSP90B 1)), in combination with an immunotherapy.

### CROSS-REFERENCE TO RELATED APPLICATIONS

This application claims the benefit of U.S. Provisional Application No. 62/532,589, filed on 14 Jul. 2017; the entire contents of said application are incorporated herein in their entirety by this reference.

### BACKGROUND OF THE INVENTION

The striking clinical success of cancer immunotherapy with checkpoint blockade suggests it is likely to form the foundation of curative therapy for many malignancies (Reck et al. (2016) N. Engl. J. Med. 375:1823-1833; Hodi et al. (2010) N. Engl. J. Med. 363:711-723; Postow et al. (2015) N. Engl. J. Med. 372:2006-2017; Wolchok et al. (2013) N. Engl. J. Med. 369:122-133; Ferris et al. (2016) N. Engl. J. Med. 375:1856-1867; Brahmer et al. (2012) N. Engl. J. Med. 366:2455-2465; Nghiem et al. (2016) N. Engl. J. Med. 374:2542-2552; Topalian et al. (2012) N. Engl. J. Med. 366:2443-2454); Motzer et al. (2015) N. Engl. J. Med. 373:1803-1813). However, despite these successes, checkpoint blockade does not achieve sustained clinical response in most patients (Tumeh et al. (2014) Nature 515:568-571; Kelderman et al. (2014) Mol. Oncol. 8:1132-1139; Zaretsky et al. (2016) N. Engl. J. Med. 375:819-829). Additional therapeutic strategies are therefore needed to increase the clinical efficacy of immunotherapy. Moreover, the optimal strategy for combining emerging cancer immunotherapies with checkpoint blockade remains uncertain.

A relatively small number of genes, such as PD-L1, that enable tumors to evade the immune system have been discovered and most of these are already the focus of intense efforts to develop new immunotherapies (Freeman et al. (2000) J. Exp. Med. 192:1027-1034; Hirano et al. (2005) Cancer Res. 65:1089-1096; Dong et al. (2002) Nat. Med. 8:793-800; Balachandran et al. (2011) Nat. Med. 17:1094-1100; Spranger et al. (2013) Sci Transl Med. 5:200ra116; Holmgaard et al. (2013) J. Exp. Med. 210:1389-1402; Sockolosky et al. (2016) Proc. Natl. Acad. Sci. U.S.A. 113:E2646-654; Liu et al. (2015) Nat. Med. 21:1209-1215; Weiskopf et al. (2016) J. Clin. Invest. 126:2610-2620; Tseng et al. (2013) Proc. Natl. Acad. Sci. U.S.A. 110: 11103-11108; Sica et al. (2003) Immunity 18:849-861; Zang et al. (2007) Proc. Natl. Acad. Sci. U.S.A. 104, 19458-19463). Although cancer cells could, in theory, express many more genes that regulate their response or resistance to tumor immunity, strategies to systematically discover such genes are lacking.

Loss-of-function genetic screens have been increasingly used to study the functional consequences of gene deletion on tumor cells (Howard et al. (2016) Functional Genomic Characterization of Cancer Genomes. Cold Spring Harb. Symp. Quant. Biol. (2016); Ebert et al. (2008) Nature 451:335-339; Cowley et al. (2014) Scientific Data 1:article number 140035). These approaches include pooled genetic screens using CRISPR-Cas9-mediated genome editing that simultaneously test the role of a large number of genes on tumor cell growth, viability or drug resistance (Wang et al. (2014) Science 343:80-84; Shalem et al. (2014) Science 343:84-87). However, these screens have generally been conducted in vitro, where the contribution of the immune system is absent, or have studied phenotypes such as metastasis that do not directly evaluate the role of tumor immunity (Hart et al. (2015) Cell 163:1515-1526; Yu et al. (2016) Nat. Biotechnol. 34:419-423; Chen et al. (2015) Cell 160:1246-1260).

Despite the dramatic clinical success of cancer immunotherapy with PD-1 checkpoint blockade, most patients do not experience sustained clinical benefit from treatment. Accordingly a great need in the art exists for additional therapeutic strategies.

### SUMMARY OF THE INVENTION

The present invention is based, at least in part, on the discovery that inhibiting or blocking one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), in combination with an immunotherapy, results in a synergistic therapeutic benefit for treating cancers that is unexpected given the lack of such benefit observed for the immunotherapy alone.

In one aspect, a method of treating a subject afflicted with a cancer comprising administering to the subject a therapeutically effective amount of an agent that inhibits the copy number, the expression level, and/or the activity of one or more biomarkers listed in Table 1 or a fragment thereof, in combination with an immunotherapy, is provided.

Numerous embodiments are further provided that can be applied to any aspect of the present invention and/or combined with any other embodiment described herein. For example, in one embodiment, the agent described herein decreases the copy number, the expression level, and/or the activity of one or more biomarkers in Table 1 (e.g., one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1))). In another embodiment, the agent selectively decreases the activity of one or more biomarkers in Table 1, such as decreasing the ubiquitin ligase activity and/or the substrate binding activity of one or more regulators of ubiquitin proteasome pathwasy (e.g., STUB1, UBQLN1, and HSP90B1).

In one embodiment, the agent described herein is a small molecule inhibitor, CRISPR single-guide RNA (sgRNA), RNA interfering agent, antisense oligonucleotide, peptide or peptidomimetic inhibitor, aptamer, or intrabody. In another embodiment, the RNA interfering agent is a small interfering RNA (siRNA), CRISPR RNA (crRNA), a small hairpin RNA (shRNA), a microRNA (miRNA), or a piwi-interacting RNA (piRNA). In still another embodiment, the RNA interfering agent is a CRISPR single-guide RNA (sgRNA). In yet another embodiment, the sgRNA comprises a nucleic acid sequence selected from the group consisting of nucleic acid sequence listed in Table 2. In one embodiment, the agent described herein comprises an intrabody, or an antigen binding fragment thereof, which specifically binds to the one or more biomarkers in Table 1 and/or a substrate of the one or more biomarkers in Table 1. In another embodiment, the intrabody, or antigen binding fragment thereof, is murine, chimeric, humanized, composite, or human. In still another embodiment, the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabodies fragments. In yet another embodiment, the intrabody, or antigen binding fragment thereof, is conjugated to a cytotoxic agent. In one embodiment, the cytotoxic agent is selected from the group consisting of a chemotherapeutic agent, a biologic agent, a toxin, and a radioactive isotope. In still another embodiment, the agent described herein increases the sensitivity of the cancer cells to an immunotherapy. In another embodiment, the immunotherapy and/or a cancer therapy is administered before, after, or concurrently with the agent. In still another embodiment, the immunotherapy comprises an anti-cancer vaccine and/or virus. In yet embodiment, the immunotherapy is cell-based. In one embodiment, immunotherapy inhibits an immune checkpoint. In another embodiment, the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, TLT-4, TIGIT, HHLA2, butyrophilins, IDO, CD39, CD73 and A2aR. In still another embodiment, the immune checkpoint is selected from the group consisting of PD-1, PD-L1, and PD-L2. In yet another embodiment, the immune checkpoint is PD-1. In one embodiment, the one or more biomarker described herein comprises a nucleic acid sequence having at least 95% identity to a nucleic acid sequence listed in Table 1 and/or encodes an amino acid sequence having at least 95% identity to an amino acid sequence listed in Table 1. In another embodiment, the one or more biomarker is human, mouse, chimeric, or a fusion. In still another embodiment, the agent reduces the number of proliferating cells in the cancer and/or reduces the volume or size of a tumor comprising the cancer cells. In another embodiment, the agent increases the sensitivity of the cancer to the immunotherapy. In still another embodiment, the one or more biomarkers comprise an amino acid sequence listed in Table 1, optionally wherein the amino acid sequence is selected from the group consisting of SEQ ID Nos: 2, 4, 6, 8, 10, 12, 14, 16, and 18. In yet another embodiment, the one or more biomarkers are encoded by a nucleic acid sequence listed in Table 1, optionally wherein the nucleic acid sequence is selected from the group consisting of SEQ ID Nos: 1, 3, 5, 7, 9, 11, 13, 15, and 17. In one embodiment, the cancer is melanoma. In another embodiment, the subject is an animal model of the cancer, preferably a mouse model, or a human. In still another embodiment, the method described herein further comprises administering to the subject at least one additional cancer therapy or regimen. In another embodiment, the at least one additional cancer therapy or regimen is administered before, after, or concurrently with the agent and/or the immunotherapy. In yet another embodiment, the agent described herein is administered in a pharmaceutically acceptable formulation.

In another aspect, a method of killing cancer cells comprising contacting the cancer cells with an agent that inhibits the copy number, the expression level, and/or the activity of one or more biomarkers listed in Table 1 or a fragment thereof, in combination with an immunotherapy, is provided.

As described above, numerous embodiments are further provided that can be applied to any aspect of the present invention and/or combined with any other embodiment described herein. For example, in one embodiment, the agent described herein decreases the copy number, the expression level, and/or the activity of one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)). In another embodiment, the agent selectively decreases activity of one or more biomarkers in Table 1, such as decreasing the ubiquitin-proteasome pathway regulating activity and/or the substrate binding activity of one or more kinase signaling inhibitors. In still another embodiment, the agent is a small molecule inhibitor, CRISPR single-guide RNA (sgRNA), RNA interfering agent, antisense oligonucleotide, peptide or peptidomimetic inhibitor, aptamer, or intrabody. In one embodiment, the RNA interfering agent described herein is a small interfering RNA (siRNA), CRISPR RNA (crRNA), a small hairpin RNA (shRNA), a microRNA (miRNA), or a piwi-interacting RNA (piRNA). In another embodiment, the RNA interfering agent is a CRISPR single-guide RNA (sgRNA). In still another embodiment, the sgRNA comprises a nucleic acid sequence selected from the group consisting of nucleic acid sequence listed in Table 2. In one embodiment, the agent described herein comprises an intrabody, or an antigen binding fragment thereof, which specifically binds to the one or more biomarkers in Table 1 and/or a substrate of the one or more biomarkers in Table 1. In another embodiment, the intrabody, or antigen binding fragment thereof, is murine, chimeric, humanized, composite, or human. In still another embodiment, the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabodies fragments. In yet another embodiment, the intrabody, or antigen binding fragment thereof, is conjugated to a cytotoxic agent. In one embodiment, the cytotoxic agent is selected from the group consisting of a chemotherapeutic agent, a biologic agent, a toxin, and a radioactive isotope. In one embodiment, the agent described herein increases the sensitivity of the cancer cells to an immunotherapy. In another embodiment, the cancer cells are contacted with an immunotherapy and/or a cancer therapy before, after, or concurrently with the agent. In still another embodiment, the immunotherapy comprises an anti-cancer vaccine and/or virus. In one embodiment, the immunotherapy is cell-based. In another embodiment, the immunotherapy inhibits an immune checkpoint. In still another embodiment, the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR. In yet another embodiment, the immune checkpoint is selected from the group consisting of PD-1, PD-L1, and PD-L2. In one embodiment, the immune checkpoint is PD-1. In another embodiment, the biomarker described herein comprises a nucleic acid sequence having at least 95% identity to a nucleic acid sequence listed in Table 1 and/or encodes an amino acid sequence having at least 95% identity to an amino acid sequence listed in Table 1. In still another embodiment, the one or more biomarker is human, mouse, chimeric, or a fusion. In one embodiment, the agent described herein reduces the number of proliferating cells in the cancer and/or reduces the volume or size of a tumor comprising the cancer cells. In another embodiment, the agent increases the sensitivity of the cancer to the immunotherapy. In still another embodiment, the one or more biomarkers comprise an amino acid sequence listed in Table 1, optionally wherein the amino acid sequence is selected from the group consisting of SEQ ID Nos: 2, 4, 6, 8, 10, 12, 14, 16, and 18. In yet another embodiment, the one or more biomarkers are encoded by a nucleic acid sequence listed in Table 1, optionally wherein the nucleic acid sequence is selected from the group consisting of SEQ ID Nos: 1, 3, 5, 7, 9, 11, 13, 15, and 17. In one embodiment, the cancer is melanoma. In another embodiment, the subject is an animal model of the cancer, preferably a mouse model, or a human. In still another embodiment, the method described herein further comprises administering to the subject at least one additional cancer therapy or regimen. In yet another embodiment, the at least one additional cancer therapy or regimen is administered before, after, or concurrently with the agent and/or the immunotherapy. In another embodiment, the agent described herein is administered in a pharmaceutically acceptable formulation.

In still another aspect, a method of determining whether a subject afflicted with a cancer or at risk for developing a cancer would benefit from inhibiting the copy number, amount, and/or activity of at least one biomarker listed in Table 1 is provided, the method comprising a) obtaining a biological sample from the subject; b) determining the copy number, amount, and/or activity of at least one biomarker listed in Table 1; c) determining the copy number, amount, and/or activity of the at least one biomarker in a control; and d) comparing the copy number, amount, and/or activity of the at least one biomarker detected in steps b) and c); wherein the presence of, or a significant increase in, the copy number, amount, and/or activity of, the at least one biomarker listed in Table 1 in the subject sample relative to the control copy number, amount, and/or activity of the at least one biomarker indicates that the subject afflicted with the cancer or at risk for developing the cancer would benefit from inhibiting the copy number, amount, and/or activity of the at least one biomarker listed in Table 1.

As described above, numerous embodiments can be applied to any aspect of the present invention. For example, in one embodiment, the method described herein further comprises recommending, prescribing, or administering an agent that inhibits the at least one biomarker listed in Table 1 if the cancer is determined to benefit from the agent. In another embodiment, the method described herein further comprises administering at least one additional cancer therapy that is administered before, after, or concurrently with the agent. In still another embodiment, the method described herein further comprises recommending, prescribing, or administering cancer therapy other than an agent that inhibits the at least one biomarker listed in Table 1 if the cancer is determined to not benefit from the agent. In yet another embodiment, the cancer therapy is selected from the group consisting of immunotherapy, targeted therapy, chemotherapy, radiation therapy, hormonal therapy, an anti-cancer vaccine, an anti-cancer virus, and a checkpoint inhibitor. In one embodiment, the control sample is determined from a cancerous or non-cancerous sample from either the patient or a member of the same species to which the patient belongs. In another embodiment, the control sample comprises cells.

In yet another aspect, a method for predicting the clinical outcome of a subject afflicted with a cancer expressing one or more biomarkers listed in Table 1 or a fragment thereof to treatment with an immunotherapy is provided, the method comprising a) determining the copy number, amount, and/or activity of at least one biomarker listed in Table 1 in a subject sample; b) determining the copy number, amount, and/or activity of the at least one biomarker in a control having a good clinical outcome; and c) comparing the copy number, amount, and/or activity of the at least one biomarker in the subject sample and in the control; wherein the presence of, or a significant increase in, the copy number, amount, and/or activity of, the at least one biomarker listed in Table 1 in the subject sample as compared to the copy number, amount and/or activity in the control, is an indication that the subject has a poor clinical outcome.

In another aspect, a method for monitoring the progression of a cancer in a subject, wherein the subject is administered a therapeutically effective amount of an agent that inhibits the copy number, amount, and/or activity of at least one biomarker listed in Table 1 and an immunotherapy is provided, the method comprising a) detecting in a subject sample at a first point in time the copy number, amount, and/or activity of at least one biomarker listed in Table 1; b) repeating step a) at a subsequent point in time; and c) comparing the amount or activity of at least one biomarker listed in Table 1 detected in steps a) and b) to monitor the progression of the cancer in the subject.

In still another aspect, a method of assessing the efficacy of an agent that inhibits the copy number, amount, and/or activity of at least one biomarker listed in Table 1 and an immunotherapy for treating a cancer in a subject is provided, comprising a) detecting in a subject sample at a first point in time the copy number, amount, and/or or activity of at least one biomarker listed in Table 1; b) repeating step a) during at least one subsequent point in time after administration of the agent and the immunotherapy; and c) comparing the copy number, amount, and/or activity detected in steps a) and b), wherein the absence of, or a significant decrease in, the copy number, amount, and/or activity of, the at least one biomarker listed in Table 1, in the subsequent sample as compared to the copy number, amount, and/or activity in the sample at the first point in time, indicates that the agent and immunotherapy treats the cancer in the subject. In one embodiment, between the first point in time and the subsequent point in time, the subject has undergone treatment, completed treatment, and/or is in remission for the cancer. In another embodiment, the cancer treatment is selected from the group consisting of immunotherapy, targeted therapy, chemotherapy, radiation therapy, hormonal therapy, an anti-cancer vaccine, an anti-cancer virus, and a checkpoint inhibitor. In still another embodiment, the first and/or at least one subsequent sample is selected from the group consisting of ex vivo and in vivo samples. In yet another embodiment, the first and/or at least one subsequent sample is a portion of a single sample or pooled samples obtained from the subject.

As described above, numerous embodiments can be applied to any aspect of the present invention. For example, in one embodiment, the sample described herein comprises cells, serum, peritumoral tissue, and/or intratumoral tissue obtained from the subject. In another embodiment, the one or more biomarkers listed in Table 1 comprise STUB1, UBQLN1, and/or HSP90B1. In still another embodiment, the cancer is melanoma. In yet another embodiment, the cancer is in a subject and the subject is a mammal. In one embodiment, the mammal is a mouse or a human. In another embodiment, the mammal is a human.

In still another aspect, an agent that inhibits one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), is provided for treating a cancer in a subject, in combination with an immunotherapy. Such agent may comprise a small molecule inhibitor, an RNA interfering agent, an antisense oligonucleotide, a peptide or peptidomimetic inhibitor, an aptamer, and/or an intrabody, as described herein.

In yet another aspect, a vector comprising an agent that inhibits one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), for treating a cancer in a subject, in combination with an immunotherapy, is provided.

In another aspect, a host cell which comprises an agent that inhibits one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), for treating a cancer in a subject, in combination with an immunotherapy, is provided.

In still another aspect, a host cell which comprises a vector comprising an agent that inhibits one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), for treating a cancer in a subject, in combination with an immunotherapy, is provided.

In yet another aspect, a device or kit comprising the agent that inhibits one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), for treating a cancer in a subject, in combination with an immunotherapy, is provided.

### BRIEF DESCRIPTION OF THE DRAWINGS

FIG. 1 includes 6 panels, identified as panels A, B, C, D, E, and F, which show that in vivo pooled loss-of-function screening using CRISPR/Cas9 in tumor cells recovers known mediators of immune evasion. Panel A shows a schematic diagram of the in vivo screening system using the B16 transplantable tumor model. Tumor volumes (in mm³) were compared under each conditions, averaged for each group at each time point (Panel B, left) or for individual animals on the day of sacrifice (Panel B, right). Bars represent means, while whiskers represent standard deviation. Enrichment analysis was carried out using a hypergeometric test to show functional classes of genes, (from the Gene Ontology Consortium database (GO)) targeted by sgRNAs, that were enriched or depleted in tumors in animals, including animals treated with irradiated tumor cell vaccine (GVAX) and anti-PD-1 antibody and the TCRα^{-/-} animals (Panel C). Frequency histogram (Panel D, top) and collapsed histograms (Panel D, middle) of enrichment or depletion (normalized as Z scores) are shown for all 9,992 sgRNAs screened. Enrichment/depletion scores are averaged from 10 mice per condition. sgRNAs targeting PD-L1 are indicated by the red lines (Panel D, middle). PD-L1 expression is compared among Cas9-expressing B16 tumor cells transfected with one of the four sgRNAs targeting PD-L1 (red) or a control sgRNA (grey) (Panel D, bottom). Similar to Panel D, Panel E shows the depletion of CD47 by its specific sgRNAs (indicated in red (top and middle) and CD47 expression after CRISPR editing with sgRNAs targeting CD47 (bottom). Panel F compares tumor volumes over time between CD47 null (red) and control (grey) tumors growing in mice treated with GVAX and PD-1 blockade (average and standard error of the mean; n=10 animals per group). ** p<0.01; ***p<0.001; ****p<0.0001.

FIG. 2 includes 6 panels, identified as panels A, B, C, D, E, and F, which show the performance analysis of the screening in FIG. 1. Panel A shows Western blot of B16 cell lysate for Cas9 and β-ACTIN with or without transduction with a lentiviral vector encoding Cas9. A pie chart shows the fraction of genes targeted in the screening in each of the GO term categories indicated (Panel B). Two-dimensional histograms show the pair-wise distribution of sgRNAs abundance (averaged for each condition) (Panel C). Saturation analysis of animal replicates from the three in vivo screening conditions is shown in Panel D. Pearson correlations are calculated for the library distribution in one animal vs. any other animal, then for two animals averaged versus any other two averaged, and so on. Saturation approaches r=0.95. A matrix of the Pearson correlations of the library distribution from one animal compared to any other animal for B16 Pool 1 is shown (Panel E). Expression of CD47 by B16 cells transfected with either CD47-targeting (red) or control (grey) sgRNA is compared (Panel F).

FIG. 3 includes 3 panels, identified as panels A, B, and C, which show that loss of ubiquitin proteasome pathway genes (e.g., Stub1) causes resistance to immunotherapy. Panel A shows frequency histogram (top) and collapsed histograms (below) of enrichment or depletion (normalized as z scores) for all sgRNAs in GVAX+PD-1 blockade-treated mice relative to TCRα^{-/-} mice, as in FIG. 1. Red bars indicate the sgRNAs for the genes listed on the left. Representative flow plots show the frequencies of control or Stub1 null B16 cells for the conditions indicated (Panel B). Specifically, mixtures of Stub1 null tumors and control tumors (in a 1:10 ratio) were tested in vitro, in TCRα^{-/-} mice, or GVAX+PD-1 blockade-treated wild-type mice. The B16 cell numbers in immunotherapy-treated wild-type mice (treated with different sgRNAs) were compared relative to those in TCRα^{-/-} mice, the change in the ratios (log 2 normalized fold change) is shown in Panel C (mean and standard deviation; n=8-10 mice per group). ***p<0.001.

### DETAILED DESCRIPTION OF THE INVENTION

It has been determined herein that negative regulators of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), can be used to increase interferon sensing by tumor cells and augment tumor immunity and immunotherapies. Thus, the instant disclosure provides at least a method of treating cancers, e.g., those cancer types otherwise not responsive or weakly responsive to immunotherapies, with a combination of a negative regulator of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and another immunotherapy. The results described herein are unexpected given that analyses of STUB1, UBQLN1, and HSP90B1 function has heretofore been largely confined to hematopoietic cells and not examined in cancer cells, as well as the fact that modulating sensitivity to immunotherapy is critical for immunotherapy effects rather than simply modulating interferon availability since interferon therapy is known to not significantly augment immunotherapy effects. Accordingly, the present invention provides exemplary RNA interfering agents inhibiting one or more regulators of ubiquitin proteasome pathway (e.g., STUB1, ubiquilin-1, and HSP90B1), which may be used in the combination therapy and other methods described herein, such as agents that inhibit biomarker function and/or its ability to interact/bind to its substrates described herein, or by increasing its degradation and/or stability and/or interaction/binding to its inhibitors. Similarly, methods of screening for biomarker inhibitors and methods of diagnosing, prognosing, and monitoring cancer involving biomarker/immunotherapy combination therapies are provided.

### I. Definitions

The articles “a” and “an” are used herein to refer to one or to more than one (i.e. to at least one) of the grammatical object of the article. By way of example, “an element” means one element or more than one element.

The term “altered amount” or “altered level” refers to increased or decreased copy number (e.g., germline and/or somatic) of a biomarker nucleic acid, e.g., increased or decreased expression level in a cancer sample, as compared to the expression level or copy number of the biomarker nucleic acid in a control sample. The term “altered amount” of a biomarker also includes an increased or decreased protein level of a biomarker protein in a sample, e.g., a cancer sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “altered level of expression” of a biomarker refers to an expression level or copy number of the biomarker in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least 20%, 30%, 40%, 50%, 60%, 70%, 80%, 90%, 100%, 150%, 200%, 300%, 350%, 400%, 500%, 600%, 700%, 800%, 900%, 1000% or more times the expression level or copy number of the biomarker in a control sample (e.g., sample from a healthy subjects not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker).

The term “altered activity” of a biomarker refers to an activity of the biomarker which is increased or decreased in a disease state, e.g., in a cancer sample, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

Unless otherwise specified here within, the terms “antibody” and “antibodies” refers to antigen-binding portions adaptable to be expressed within cells as “intracellular antibodies.” (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

Antibodies may be polyclonal or monoclonal; xenogeneic, allogeneic, or syngeneic; or modified forms thereof (e.g. humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “assigned score” refers to the numerical value designated for each of the biomarkers after being measured in a patient sample. The assigned score correlates to the absence, presence or inferred amount of the biomarker in the sample. The assigned score can be generated manually (e.g., by visual inspection) or with the aid of instrumentation for image acquisition and analysis. In certain embodiments, the assigned score is determined by a qualitative assessment, for example, detection of a fluorescent readout on a graded scale, or quantitative assessment. In one embodiment, an “aggregate score,” which refers to the combination of assigned scores from a plurality of measured biomarkers, is determined. In one embodiment the aggregate score is a summation of assigned scores. In another embodiment, combination of assigned scores involves performing mathematical operations on the assigned scores before combining them into an aggregate score. In certain, embodiments, the aggregate score is also referred to herein as the “predictive score.”

The term “biomarker” refers to a measurable entity of the present invention that has been determined to be predictive of combinatorial therapy effects on a cancer, in which the combinatory therapy comprising inhibitors of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and immunotherapy (e.g., immune checkpoint inhibitors). Biomarkers can include, without limitation, nucleic acids and proteins, including those shown in the Tables, the Examples, the Figures, and otherwise described herein. As described herein, any relevant characteristic of a biomarker can be used, such as the copy number, amount, activity, location, modification (e.g., phosphorylation), and the like.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. Unless otherwise stated, the terms include metaplasias. In some embodiments, such cells exhibit such characteristics in part or in full due to the expression and activity of one or more biomarkers listed in Table 1-regulated signaling pathways (e.g., ubiquitin proteasome pathway, unfolded protein response, CXCR4-mediated signaling events, vesicle-mediated transport, clathrin-mediated endocytosis, NF-kappaB Signaling pathway, MAP kinase pathway, JAK-STAT signaling pathway, or other signaling pathways). In some embodiments, the cancer cells described herein are not sensitive to at least one of immunotherapies. Such insensitivity, without limitation, may be related to the inactivation or decreased activation, compared to control cells (e.g., normal and/or wild-type non-cancer cells, and/or cancer cells without this insensitivity to immunotherapies), of interferon signaling (e.g., IFNγ signaling) in such cancer cells and/or other surrounding cells and/or cells localized near to such cancer cells. In some embodiments, the cancer cells are treatable with an agent capable of antagonizing one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)) In some embodiments, the treatment with the agent antagonizing one or more biomarkers listed in Table 1 as described herein would increase IFNγ signaling in such cancer cells, compared to pre-treatment situations, or would restore IFNγ signaling in such cancer cells to at least comparable to the levels in control cells, so that such cancer cells would regain sensitivity to immunotherapies. The term “interferon signaling” or “IFNγ signaling” used herein refers to any cell signaling downstream and/or related to the interaction of interferon (e.g., IFNγ) and their receptor(s). Some exemplary IFNγ cell signaling include, without limitation, the activation of macrophages and/or induction of Class II major histocompatibility complex (MHC) molecule expression, and/or activation of multiple immune effector genes through the Janus kinase (JAK)-STAT signaling pathway (e.g., through STAT1 transcription factor).

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

In certain embodiments, the cancer encompasses colorectal cancer (e.g., colorectal carcinoma).

The term “colorectal cancer” as used herein, is meant to include cancer of cells of the intestinal tract below the small intestine (e.g., the large intestine (colon), including the cecum, ascending colon, transverse colon, descending colon, and sigmoid colon, and rectum). Additionally, as used herein, the term “colorectal cancer” is meant to further include cancer of cells of the duodenum and small intestine (jejunum and ileum). Colorectal cancer also includes neoplastic diseases involving proliferation of a single clone of cells of the colon and includes adenocarcinoma and carcinoma of the colon whether in a primary site or metastasized.

Colorectal cancer (CRC) is the third most commonly diagnosed cancer and ranks second in cancer mortality. Extensive genetic and genomic analysis of human CRC has uncovered germline and somatic mutations relevant to CRC biology and malignant transformation (Fearon et al. (1990) Cell 61, 759-767). These mutations have been linked to well-defined disease stages from aberrant crypt proliferation or hyperplasic lesions to benign adenomas, to carcinoma in situ, and finally to invasive and metastatic disease, thereby establishing a genetic paradigm for cancer initiation and progression. Genetic and genomic instability are catalysts for colon carcinogenesis (Lengauer et al. (1998) Nature 396:643-649). CRC can present with two distinct genomic profiles that have been termed (i) chromosomal instability neoplasia (CIN), characterized by rampant structural and numerical chromosomal aberrations driven in part by telomere dysfunction (Artandi et al. (2000) Nature 406:641-645; Fodde et al. (2001) Nat. Rev. Cancer 1:55-67; Maser and DePinho (2002) Science 297:565-569; Rudolph et al. (2001) Nat. Genet. 28:155-159) and mitotic aberrations (Lengauer et al. (1998) Nature 396:643-649) and (ii) microsatellite instability neoplasia (MIN), characterized by near diploid karyotypes with alterations at the nucleotide level due to mutations in mismatch repair (MMR) genes (Fishel et al. (1993) Cell 75:1027-1038; Ilyas et al. (1999) Eur. J. Cancer 35:335-351; Modrich (1991) Annu. Rev. Genet. 25:229-253; Parsons et al. (1995) Science 268:738-740; Parsons et al. (1993) Cell 75:1227-1236). Germline MMR mutations are highly penetrant lesions which drive the MIN phenotype in hereditary nonpolyposis colorectal cancers, accounting for 1-5% of CRC cases (de la Chapelle (2004) Nat. Rev. Cancer 4:769-780; Lynch and de la Chapelle (1999) J. Med. Genet. 36:801-818; Umar et al. (2004) Nat. Rev. Cancer 4:153-158). While CIN and MIN are mechanistically distinct, their genomic and genetic consequences emphasize the requirement of dominant mutator mechanisms to drive intestinal epithelial cells towards a threshold of oncogenic changes needed for malignant transformation.

A growing number of genetic mutations have been identified and functionally validated in CRC pathogenesis. Activation of the WNT signaling pathway is an early requisite event for adenoma formation. Somatic alterations are present in APC in greater than 70% of nonfamilial sporadic cases and appear to contribute to genomic instability and induce the expression of c-myc and Cyclin D1 (Fodde et al. (2001) Nat. Rev. Cancer 1:55-67), while activating β-catenin mutations represent an alternative means of WNT pathway deregulation in CRC (Morin (1997) Science 275:1787-1790). K-Ras mutations occur early in neoplastic progression and are present in approximately 50% of large adenomas (Fearon and Gruber (2001) Molecular abnormalities in colon and rectal cancer, ed. J. Mendelsohm, P. H., M. Israel, and L. Liotta, W.B. Saunders, Philadelphia). The BRAF serine/threonine kinase and PIK3CA lipid kinase are mutated in 5-18% and 28% of sporadic CRCs, respectively (Samuels et al. (2004) Science 304:554; Davies et al. (2002) Nature 417:949-954; Rajagopalan et al. (2002) Nature 418:934; Yuen et al. (2002) Cancer Res. 62:6451-6455). BRAF and K-ras mutations are mutually exclusive in CRC, suggesting over-lapping oncogenic activities (Davies et al. (2002) Nature 417:949-954; Rajagopalan et al. (2002) Nature 418:934). Mutations associated with CRC progression, specifically the adenoma-to-carcinoma transition, target the TP53 and the TGF-β pathways (Markowitz et al. (2002) Cancer Cell 1:233-236). Greater than 50% of CRCs harbor TP53 inactivating mutations (Fearon and Gruber (2001) Molecular abnormalities in colon and rectal cancer, ed. J. Mendelsohm, P. H., M. Israel, and L. Liotta, W.B. Saunders, Philadelphia) and 30% of cases possess TGFβ-RII mutations (Markowitz (2000) Biochim. Biophys. Acta 1470:M13-M20; Markowitz et al. (1995) Science 268:1336-1338). MIN cancers consistently inactivate TGFβ-RII by frameshift mutations, whereas CIN cancers target the pathway via inactivating somatic mutations in the TGFβ-RII kinase domain (15%) or in the downstream signaling components of the pathway, including SMAD4 (15%) or SMAD2 (5%) transcription factors (Markowitz (2000) Biochim. Biophys. Acta 1470:M13-M20). In some embodiments, the colorectal cancer is microsatellite instable (MSI) colorectal cancer (Llosa et al. (2014) Cancer Disc. CD-14-0863; published online Oct. 30, 2014). MSI represents about 15% of sporadic CRC and about 5-6% of stage IV CRCs. MSI is caused by epigenetic silencing or mutation of DNA mismatch repair genes and typically presents with lower stage disease than microsatellite stable subset (MSS) CRC. MSI highly express immune checkpoints, such as PD-1, PD-L1, CTLA-4, LAG-3, and IDO. In other embodiments, the colorectal cancer is MSS CRC.

In certain embodiments, the cancer encompasses melanoma. The term “melanoma” as used herein, is generally meant to include cancers that develop from the pigment-containing cells, known as melanocytes, in the basal layer of the epidermis. Melanomas typically occur in the skin but may rarely occur in the mouth, intestines, or eye. In women they most commonly occur on the legs, while in men they are most common on the back. Sometimes they develop from a mole with concerning changes including an increase in size, irregular edges, change in color, itchiness, or skin breakdown. Thus, the term “melanoma” also includes cancers developing from these cells, tissues, and organs.

Melanomas are among the most dangerous forms of skin cancer and develop when unrepaired DNA damage to skin cells (most often caused by ultraviolet radiation from sunshine or tanning beds) triggers gene mutations that lead the skin cells to multiply rapidly and form malignant tumors. The primary cause of melanoma is ultraviolet light (UV) exposure in those with low levels of skin pigment. Melanomas often resemble moles; some develop from moles. Those with many moles, a history of affected family members, and who have poor immune function are at greater risk. A number of rare genetic defects such as xeroderma pigmentosum also increase risk (Azoury and Lange, 2014 Surg Clin North Am. 2014 94:945-962).

Melanoma can be divided into different types, including, at least, lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, melanoma with small nevus-like cells, melanoma with features of a Spitz nevus, uveal melanoma, etc. (see James, et al., 2006 Andrews' Diseases of the Skin: clinical Dermatology. Saunders Elsevier. pp. 694-9) Diagnosis is by biopsy of any concerning skin lesion, including, at least, shave (tangential) biopsy, punch biopsy, incisional and excisional biopsies, “optical” biopsies (e.g., by reflectance confocal microscopy (RCM)), fine needle aspiration (FNA) biopsy, surgical lymph node biopsy, sentinel lymph node biopsy, etc. In addition, visual inspection may also be used for diagnosis, such as a popular method for the signs and symptoms of melanoma as mnemonic “ABCDE”: Asymmetrical skin lesion, Border of the lesion is irregular, Color: melanomas usually have multiple colors, Diameter: moles greater than 6 mm are more likely to be melanomas than smaller moles, and Enlarging: Enlarging or evolving. Another method as the “ugly duckling sign” is also known in the art (Mascaro and Mascaro, 1998 Arch Dermatol. 134: 1484-1485).

Treatment of melanoma includes surgery, chemotherapy (such as temozolomide, dacarbazine (also termed DTIC), etc.), radiation therapy, oncolytic virotherapy (e.g., see Forbes et al., 2013 Front. Genet. 4:184), and immunotherapy (e.g., interleukin-2 (IL-2), interferon, etc.). Targeted therapies (e.g., as in Maverakis et al., 2015 Acta Derm Venereol. 95: 516-524) may include: 1) adoptive cell therapy (ACT) using TILs immune cells (tumor infiltrating lymphocytes) isolated from a person's own melanoma tumor). Cells are grown in large numbers in a laboratory and returned to the patient after a treatment that temporarily reduces normal T cells in the patient's body. TIL therapy following lymphodepletion can result in durable complete response in a variety of setups (Besser et al., 2010 Clin. Cancer Res. 16:2646-2655); and 2) adoptive transfer of genetically altered (expressing T cell receptors (TCRs)) autologous lymphocytes into patient's lymphocytes, where the altered lymphocytes recognize and bind to the surface of melanoma cells and kill them. Other therapies include, at least, B-Raf inhibitors (such as vemurafenib, see Chapman et al., 2011 N. Engl. J Med. 364:2507-2516) and ipilimumab (alone or in combination with dacarbazine, see, e.g., Robert et al. (2011) N. Engl. J. Med. 364:2517-2526).

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the present invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid or protein is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with cancer, or from a corresponding non-cancerous tissue in the same subject who has cancer.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal can result in cytokine secretion, e.g., in a T cell that has received a T cell-receptor-mediated signal. Immune cells that have received a cell-receptor mediated signal, e.g., via an activating receptor are referred to herein as “activated immune cells.”

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “diagnosing cancer” includes the use of the methods, systems, and code of the present invention to determine the presence or absence of a cancer or subtype thereof in an individual. The term also includes methods, systems, and code for assessing the level of disease activity in an individual.

A molecule is “fixed” or “affixed” to a substrate if it is covalently or non-covalently associated with the substrate such that the substrate can be rinsed with a fluid (e.g. standard saline citrate, pH 7.4) without a substantial fraction of the molecule dissociating from the substrate.

The term “expression signature” or “signature” refers to a group of one or more coordinately expressed biomarkers related to a measured phenotype. For example, the genes, proteins, metabolites, and the like making up this signature may be expressed in a specific cell lineage, stage of differentiation, or during a particular biological response. The biomarkers can reflect biological aspects of the tumors in which they are expressed, such as the cell of origin of the cancer, the nature of the non-malignant cells in the biopsy, and the oncogenic mechanisms responsible for the cancer. Expression data and gene expression levels can be stored on computer readable media, e.g., the computer readable medium used in conjunction with a microarray or chip reading device. Such expression data can be manipulated to generate expression signatures.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In some embodiments, the immunotherapy described herein comprises at least one of immunogenic chemotherapies. The term “immunogenic chemotherapy” refers to any chemotherapy that has been demonstrated to induce immunogenic cell death, a state that is detectable by the release of one or more damage-associated molecular pattern (DAMP) molecules, including, but not limited to, calreticulin, ATP and HMGB1 (Kroemer et al. (2013), Annu. Rev. Immunol., 31:51-72). Specific representative examples of consensus immunogenic chemotherapies include anthracyclines, such as doxorubicin and the platinum drug, oxaliplatin, 5′-fluorouracil, among others.

In some embodiments, immunotherapy comprises inhibitors of one or more immune checkpoints. The term “immune checkpoint” refers to a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein. In one embodiment, the immune checkpoint is PD-1.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

The nucleic acid and amino acid sequences of a representative human PD-1 biomarker is available to the public at the GenBank database under NM_005018.2 and NP_005009.2 and is shown in Table 1 (see also Ishida et al. (1992) 20 EMBO J 11:3887; Shinohara et al. (1994) Genomics 23:704; U.S. Pat. No. 5,698,520). PD-1 has an extracellular region containing immunoglobulin superfamily domain, a transmembrane domain, and an intracellular region including an immunoreceptor tyrosine-based inhibitory motif (ITIM) (Ishida et al. (1992) EMBO J. 11:3887; Shinohara et al. (1994) Genomics 23:704; and U.S. Pat. No. 5,698,520) and an immunoreceptor tyrosine-based switch motif (ITSM). These features also define a larger family of polypeptides, called the immunoinhibitory receptors, which also includes gp49B, PIR-B, and the killer inhibitory receptors (KIRs) (Vivier and Daeron (1997) Immunol. Today 18:286). It is often assumed that the tyrosyl phosphorylated ITIM and ITSM motif of these receptors interacts with SH2-domain containing phosphatases, which leads to inhibitory signals. A subset of these immunoinhibitory receptors bind to MHC polypeptides, for example the KIRs, and CTLA4 binds to B7-1 and B7-2. It has been proposed that there is a phylogenetic relationship between the MHC and B7 genes (Henry et al. (1999) Immunol. Today 20(6):285-8). Nucleic acid and polypeptide sequences of PD-1 orthologs in organisms other than humans are well-known and include, for example, mouse PD-1 (NM 008798.2 and NP_032824.1), rat PD-1 (NM_001106927.1 and NP_001100397.1), dog PD-1 (XM_543338.3 and XP_543338.3), cow PD-1 (NM_001083506.1 and NP_001076975.1), and chicken PD-1 (XM_422723.3 and XP_422723.2).

PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells.

The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell costimulatory or inhibitory signals, and the ability to modulate the immune response.

The term “PD-1 ligand” refers to binding partners of the PD-1 receptor and includes both PD-L1 (Freeman et al. (2000) J. Exp. Med. 192:1027-1034) and PD-L2 (Latchman et al. (2001) Nat. Immunol. 2:261). At least two types of human PD-1 ligand polypeptides exist. PD-1 ligand proteins comprise a signal sequence, and an IgV domain, an IgC domain, a transmembrane domain, and a short cytoplasmic tail. Both PD-L1 (See Freeman et al. (2000) for sequence data) and PD-L2 (See Latchman et al. (2001) Nat. Immunol. 2:261 for sequence data) are members of the B7 family of polypeptides. Both PD-L1 and PD-L2 are expressed in placenta, spleen, lymph nodes, thymus, and heart. Only PD-L2 is expressed in pancreas, lung and liver, while only PD-L1 is expressed in fetal liver. Both PD-1 ligands are upregulated on activated monocytes and dendritic cells, although PD-L1 expression is broader. For example, PD-L1 is known to be constitutively expressed and upregulated to higher levels on murine hematopoietic cells (e.g., T cells, B cells, macrophages, dendritic cells (DCs), and bone marrow-derived mast cells) and non-hematopoietic cells (e.g., endothelial, epithelial, and muscle cells), whereas PD-L2 is inducibly expressed on DCs, macrophages, and bone marrow-derived mast cells (see Butte et al. (2007) Immunity 27:111).

PD-1 ligands comprise a family of polypeptides having certain conserved structural and functional features. The term “family” when used to refer to proteins or nucleic acid molecules, is intended to mean two or more proteins or nucleic acid molecules having a common structural domain or motif and having sufficient amino acid or nucleotide sequence homology, as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics. PD-1 ligands are members of the B7 family of polypeptides. The term “B7 family” or “B7 polypeptides” as used herein includes costimulatory polypeptides that share sequence homology with B7 polypeptides, e.g., with B7-1, B7-2, B7h (Swallow et al. (1999) Immunity 11:423), and/or PD-1 ligands (e.g., PD-L1 or PD-L2). For example, human B7-1 and B7-2 share approximately 26% amino acid sequence identity when compared using the BLAST program at NCBI with the default parameters (Blosum62 matrix with gap penalties set at existence 11 and extension 1 (See the NCBI website). The term B7 family also includes variants of these polypeptides which are capable of modulating immune cell function. The B7 family of molecules share a number of conserved regions, including signal domains, IgV domains and the IgC domains. IgV domains and the IgC domains are art-recognized Ig superfamily member domains. These domains correspond to structural units that have distinct folding patterns called Ig folds. Ig folds are comprised of a sandwich of two β sheets, each consisting of anti-parallel β strands of 5-10 amino acids with a conserved disulfide bond between the two sheets in most, but not all, IgC domains of Ig, TCR, and MHC molecules share the same types of sequence patterns and are called the C1-set within the Ig superfamily. Other IgC domains fall within other sets. IgV domains also share sequence patterns and are called V set domains. IgV domains are longer than IgC domains and contain an additional pair of β strands.

Preferred B7 polypeptides are capable of providing costimulatory or inhibitory signals to immune cells to thereby promote or inhibit immune cell responses. For example, B7 family members that bind to costimulatory receptors increase T cell activation and proliferation, while B7 family members that bind to inhibitory receptors reduce costimulation. Moreover, the same B7 family member may increase or decrease T cell costimulation. For example, when bound to a costimulatory receptor, PD-1 ligand can induce costimulation of immune cells or can inhibit immune cell costimulation, e.g., when present in soluble form. When bound to an inhibitory receptor, PD-1 ligand polypeptides can transmit an inhibitory signal to an immune cell. Preferred B7 family members include B7-1, B7-2, B7h, PD-L1 or PD-L2 and soluble fragments or derivatives thereof. In one embodiment, B7 family members bind to one or more receptors on an immune cell, e.g., CTLA4, CD28, ICOS, PD-1 and/or other receptors, and, depending on the receptor, have the ability to transmit an inhibitory signal or a costimulatory signal to an immune cell, preferably a T cell.

Modulation of a costimulatory signal results in modulation of effector function of an immune cell. Thus, the term “PD-1 ligand activity” includes the ability of a PD-1 ligand polypeptide to bind its natural receptor(s) (e.g. PD-1 or B7-1), the ability to modulate immune cell costimulatory or inhibitory signals, and the ability to modulate the immune response.

The term “PD-L1” refers to a specific PD-1 ligand. Two forms of human PD-L1 molecules have been identified. One form is a naturally occurring PD-L1 soluble polypeptide, i.e., having a short hydrophilic domain and no transmembrane domain, and is referred to herein as PD-L1S. The second form is a cell-associated polypeptide, i.e., having a transmembrane and cytoplasmic domain, referred to herein as PD-L1M. The nucleic acid and amino acid sequences of representative human PD-L1 biomarkers regarding PD-L1M are also available to the public at the GenBank database under NM_014143.3 and NP_054862.1. PD-L1 proteins comprise a signal sequence, and an IgV domain and an IgC domain. The signal sequence of PD-L1S is shown from about amino acid 1 to about amino acid 18. The signal sequence of PD-L1M is shown:from about amino acid 1 to about amino acid 18. The IgV domain of PD-L1S is shown from about amino acid 19 to about amino acid 134 and the IgV domain of PD-L1M is shown from about amino acid 19 to about amino acid 134. The IgC domain of PD-L1S is shown from about amino acid 135 to about amino acid 227 and the IgC domain of PD-L1M is shown from about amino acid 135 to about amino acid 227. The hydrophilic tail of the PD-L1 exemplified in PD-L1S comprises a hydrophilic tail shown from about amino acid 228 to about amino acid 245. The PD-L1 polypeptide exemplified in PD-L1M comprises a transmembrane domain shown from about amino acids 239 to about amino acid 259 and a cytoplasmic domain shown from about 30 amino acid 260 to about amino acid 290. In addition, nucleic acid and polypeptide sequences of PD-L1 orthologs in organisms other than humans are well-known and include, for example, mouse PD-L1 (NM_021893.3 and NP_068693.1), rat PD-L1 (NM_001191954.1 and NP_001178883.1), dog PD-L1 (XM_541302.3 and XP_541302.3), cow PD-L1 (NM_001163412.1 and NP_001156884.1), and chicken PD-L1 (XM_424811.3 and XP_424811.3).

The term “PD-L2” refers to another specific PD-1 ligand. PD-L2 is a B7 family member expressed on various APCs, including dendritic cells, macrophages and bone-marrow derived mast cells (Zhong et al. (2007) Eur. J. Immunol. 37:2405). APC-expressed PD-L2 is able to both inhibit T cell activation through ligation of PD-1 and costimulate T cell activation, through a PD-1 independent mechanism (Shin et al. (2005) J. Exp. Med. 201:1531). In addition, ligation of dendritic cell-expressed PD-L2 results in enhanced dendritic cell cytokine expression and survival (Radhakrishnan et al. (2003) J. Immunol. 37:1827; Nguyen et al. (2002) J. Exp. Med. 196:1393). The nucleic acid and amino acid sequences of representative human PD-L2 biomarkers are well-known in the art and are also available to the public at the GenBank database under NM_025239.3 and NP_079515.2. PD-L2 proteins are characterized by common structural elements. In some embodiments, PD-L2 proteins include at least one or more of the following domains: a signal peptide domain, a transmembrane domain, an IgV domain, an IgC domain, an extracellular domain, a transmembrane domain, and a cytoplasmic domain. For example, amino acids 1-19 of PD-L2 comprises a signal sequence. As used herein, a “signal sequence” or “signal peptide” serves to direct a polypeptide containing such a sequence to a lipid bilayer, and is cleaved in secreted and membrane bound polypeptides and includes a peptide containing about 15 or more amino acids which occurs at the N-terminus of secretory and membrane bound polypeptides and which contains a large number of hydrophobic amino acid residues. For example, a signal sequence contains at least about 10-30 amino acid residues, preferably about 15-25 amino acid residues, more preferably about 18-20 amino acid residues, and even more preferably about 19 amino acid residues, and has at least about 35-65%, preferably about 38-50%, and more preferably about 40-45% hydrophobic amino acid residues (e.g., valine, leucine, isoleucine or phenylalanine). In another embodiment, amino acid residues 220-243 of the native human PD-L2 polypeptide and amino acid residues 201-243 of the mature polypeptide comprise a transmembrane domain. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, W. N. et al. (1996) Annu. Rev. Neurosci. 19: 235-263. In still another embodiment, amino acid residues 20-120 of the native human PD-L2 polypeptide and amino acid residues 1-101 of the mature polypeptide comprise an IgV domain. Amino acid residues 121-219 of the native human PD-L2 polypeptide and amino acid residues 102-200 of the mature polypeptide comprise an IgC domain. As used herein, IgV and IgC domains are recognized in the art as Ig superfamily member domains. These domains correspond to structural units that have distinct folding patterns called Ig folds. Ig folds are comprised of a sandwich of two 1 sheets, each consisting of antiparallel (3 strands of 5-10 amino acids with a conserved disulfide bond between the two sheets in most, but not all, domains. IgC domains of Ig, TCR, and MHC molecules share the same types of sequence patterns and are called the Cl set within the Ig superfamily. Other IgC domains fall within other sets. IgV domains also share sequence patterns and are called V set domains. IgV domains are longer than C-domains and form an additional pair of strands. In yet another embodiment, amino acid residues 1-219 of the native human PD-L2 polypeptide and amino acid residues 1-200 of the mature polypeptide comprise an extracellular domain. As used herein, the term “extracellular domain” represents the N-terminal amino acids which extend as a tail from the surface of a cell. An extracellular domain of the present invention includes an IgV domain and an IgC domain, and may include a signal peptide domain. In still another embodiment, amino acid residues 244-273 of the native human PD-L2 polypeptide and amino acid residues 225-273 of the mature polypeptide comprise a cytoplasmic domain. As used herein, the term “cytoplasmic domain” represents the C-terminal amino acids which extend as a tail into the cytoplasm of a cell. In addition, nucleic acid and polypeptide sequences of PD-L2 orthologs in organisms other than humans are well-known and include, for example, mouse PD-L2 (NM_021396.2 and NP_067371.1), rat PD-L2 (NM_001107582.2 and NP_001101052.2), dog PD-L2 (XM_847012.2 and XP_852105.2), cow PD-L2 (XM_586846.5 and XP_586846.3), and chimpanzee PD-L2 (XM_001140776.2 and XP_001140776.1).

The term “PD-L2 activity,” “biological activity of PD-L2,” or “functional activity of PD-L2,” refers to an activity exerted by a PD-L2 protein, polypeptide or nucleic acid molecule on a PD-L2-responsive cell or tissue, or on a PD-L2 polypeptide binding partner, as determined in vivo, or in vitro, according to standard techniques. In one embodiment, a PD-L2 activity is a direct activity, such as an association with a PD-L2 binding partner. As used herein, a “target molecule” or “binding partner” is a molecule with which a PD-L2 polypeptide binds or interacts in nature, such that PD-L2-mediated function is achieved. In an exemplary embodiment, a PD-L2 target molecule is the receptor RGMb. Alternatively, a PD-L2 activity is an indirect activity, such as a cellular signaling activity mediated by interaction of the PD-L2 polypeptide with its natural binding partner (i.e., physiologically relevant interacting macromolecule involved in an immune function or other biologically relevant function), e.g., RGMb. The biological activities of PD-L2 are described herein. For example, the PD-L2 polypeptides of the present invention can have one or more of the following activities: 1) bind to and/or modulate the activity of the receptor RGMb, PD-1, or other PD-L2 natural binding partners, 2) modulate intra- or intercellular signaling, 3) modulate activation of immune cells, e.g., T lymphocytes, and 4) modulate the immune response of an organism, e.g., a mouse or human organism.

“Anti-immune checkpoint therapy” refers to the use of agents that inhibit immune checkpoint nucleic acids and/or proteins. Inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy).

The term “STUB1,” also known as STIP1 homology and U-Box containing protein 1 and CHIP (C-terminus of HSC70-Interacting Protein), refers to a member of a group of proteins capable of binding to and inhibiting the ATPase activity of the chaperone proteins HSC70 and HSP70 and blocking the forward reaction of the HSC70-HSP70 substrate-binding cycle (Ballinger et al. (1999) Mol. Cell. Biol. 19:4535-4545). In addition, STUB1 possesses E3 ubiquitin ligase activity and promotes ubiquitylation (Jiang et al. (2001) J. Biol. Chem. 276:42938-42944; Murata et al. (2003) Int J Biochem Cell Biol. 35:572-578). STUB1/CHIP has a tetratricopeptide repeat (TPR) motif and a a modified RING finger (U-box) domain, while the TPR motif associates with Hsc70 and Hsp90 and the U-box domain executes a ubiquitin ligase activity. Thus, STUB1 is an ideal molecule acting as a protein quality-control ubiquitin ligase that selectively leads abnormal proteins recognized by molecular chaperones to degradation by the proteasome. STUB1 enhances HSP70 induction during acute stress and also mediates its turnover during the stress recovery process. Hence STUB1/CHIP appears to maintain protein homeostasis by controlling chaperone levels during stress and recovery (Qian et al. (2006) Nature 440:551-555). The ubiquitylation substrates of STUB1 include, at least, shock cognate 71 kDa protein (Hspa8) and DNA polymerase beta (Polb). Mutations in stub1 cause spinocerebellar ataxia, autosomal recessive 16 (Synofzik et al. (2014) Orphanet J Rare Dis 9:57). Alternative splicing results in multiple stub1 transcript variants. There is a pseudogene for stub1 on chromosome 2. STUB1 has been reported to interact with C-Raf (Dogan et al. (2008) Nat. Cell Biol. 10:1447-1455), DNAJB1 ((Ballinger et al. (1999), supra), HSPA1A (Ballinger et al. (1999), supra; Imai et al. (2002) Mol Cell. 10:55-67), HSPA4 (Ballinger et al. (1999), supra), HSPA8 (Id.), Parkin (ligase) (Imai et al. (2002), supra), and RUNX2 (Li et al. (2008) J. Cell Biol. 181:959-972).

The nucleic acid and amino acid sequences of a representative human STUB1 is available to the public at the GenBank database (Gene ID 10273) and is shown in Table 1. Human STUB1 isoforms include the longer isoform a (GenBank database numbers NM_005861.3 and NP_005852.2, encoded by the shorter transcript variant 1), and the shorter isoforms b (NM_001293197.1 and NP_001280126.1, encoded by a longer transcript variant 2, which uses an alternate splice site in the 5′ region and initiates translation at a downstream in-frame start codon, compared to variant 1. The encoded isoform (b) has a shorter N-terminus than isoform a). The domain structure of STUB1 polypeptide is well known and accessible in UniProtKB database under the accession number Q9UNE7, including, in the order from the 5′ terminus to the 3′ terminus, a tetratricopeptide repeat (TPR) motif comprising at least three TPRs, e.g., from amino acid positions 26 to 59, 60 to 93, and 95 to 127 of NP_005852.2), and a modified RING finger (U-box), e.g., from amino acid 226 to 300 of NP_005852.2. The TPR domain is essential for ubiquitination mediated by UBE2D1 (Windheim et al. (2008) Biochem. J. 409:723-729). The domain typically contains 34 amino acids, having a consensus sequence of [WLF]-X(2)-[LIM]-[GAS]-X(2)-[YLF]-X(8)-[ASE]-X(3)-[FYL]-X(2)-[ASL]-X(4)-[PKE] (domain reference # cd00189 in Conserved Domain Database (CDD) with NCBI). The TPR domain has been found in a variety of organisms including bacteria, cyanobacteria, yeast, fungi, plants, and humans in various subcellular locations, involving in a variety of functions including protein-protein interactions, chaperone, cell-cycle, transciption, and protein transport complexes (Blatch and Lassle (1999) Bioessays 21:932-939). The number of TPR motifs varies among proteins, while 5-6 tandem repeats generate a right-handed helical structure with an amphipathic channel that is thought to accomodate an alpha-helix of a target protein. It has been proposed that TPR proteins preferably interact with WD-40 repeat proteins, but in many instances several TPR-proteins seem to aggregate to multi-protein complexes. Exemplary TPR-proteins include, e.g., Cdc16p, Cdc23p, and Cdc27p components of the cyclosome/APC, the Pex5p/Pas10p receptor for peroxisomal targeting signals, the Tom70p co-receptor for mitochondrial targeting signals, Ser/Thr phosphatase 5C and the p110 subunit of O-GlcNAc transferase, etc. The U-box domain is required for the ubiquitin protein ligase activity. The U-box domain (domain reference # c117544 in Conserved Domain Database (CDD)) is related to a type of RING finger (pfam00097) structure but lacks the zinc binding residues. Pfam00097 referes to the C3HC4 type zinc-finger (RING finger) in the Protein Families Database of Alignments (Pfam) database (a database of protein families that includes their annotations and multiple sequence alignments generated using hidden Markov models). Such type of RING finger is a cysteine-rich domain of 40 to 60 residues that coordinates two zinc ions, and has the consensus sequence: C-X2-C-X(9-39)-C-X(1-3)-H-X(2-3)-C-X2-C-X(4-48)-C-X2-C where X is any amino acid. Many proteins containing a RING finger play a key role in the ubiquitination pathway.

Nucleic acid and polypeptide sequences of STUB1/CHIP orthologs in organisms other than humans are well-known and include, for example, chimpanzee (Pan troglodytes) STUB1 (XM_009430024.2 and XP_009428299.1, and XM_009430025.2 and XP_009428300.1), Rhesus monkey STUB1 (NM_001257558.1 and NP_001244487.1), dog STUB1 (XM_005621746.2 and XP_005621803.1), mouse STUB1 (NM_019719.3 and NP_062693.1), cattle STUB1 (NM_001075166.1 and NP_001068634.1), Norway rat (Rattus norvegicus) STUB1 (NM_001025625.2 and NP_001020796.2), chicken STUB1 (NM_001031406.2 and NP_001026577.1), tropical clawed frog (Xenopus tropicalis) STUB1 (NM_001078879.1 and NP_001072347.1), zebrafish (Danio rerio) STUB1 (NM_199674.2 and NP_955968.2), fruit fly (Drosophila melanogaster) STUB1 (NM_058093.4 and NP_477441.1), zebrafish (Danio rerio) STUB1 (NM_199674.2 and NP_955968.2),

The term “STUB/CHIP activity” includes the ability of a STUB1/CHIP polypeptide (and its fragments, domains, and/or motifs thereof, discussed herein) to bind its substrate and catalyze ubiquitylation of its substrates). STUB1 activity may also include one or more of functions, such as affecting substrate function in a number of ways, including promoting protein degradation of its substrate through the proteasome by facilitating the ubiquitylation of the subjrate. For example, STUB1 may ubiquitylate misfolded chaperone substrates modulate the activity of several chaperone complexes, including Hsp70, Hsc70 and Hsp90. Other reported STUB1 functions include, e.g., mediating transfer of non-canonical short ubiquitin chains to HSPA8 that have no effect on HSPA8 degradation, mediating polyubiquitylation of DNA polymerase beta (POLB) at Lys-41, Lys-61, and Lys-81, thereby playing a role in base-excision repair, catalyzing polyubiquitylation by amplifying the HUWE1/ARF-BP1-dependent monoubiquitination and leading to POLB-degradation by the proteasome, mediating polyubiquitylation of CYP3A4, ubiquitylating EPHA2 and regulating the receptor stability and activity, acting as a co-chaperone for HSPA1A and HSPA1B chaperone proteins (Seo et al. (2016) Nat. Commun. 7:12882-12882). Furthermore, STUB1 can negatively regulates the suppressive function of regulatory T-cells (Treg) during inflammation by mediating the ubiquitination and degradation of FOXP3 in a HSPA1A/B-dependent manner (Chen et al. (2013) Immunity 39:272-285). STUB1 can also negatively regulates TGF-beta signaling by modulating the basal level of SMAD3 via ubiquitin-mediated degradation (Shang et al. (2014) Biochem. Biophys. Res. Commun. 446:387-392).

The term “STUB1/CHIP substrate(s)” refers to binding partners of a STUB1/CHIP polypeptide (and its fragments, domains, and/or motifs thereof, discussed herein), e.g., the proteins listed herein. Such binding partners are usually ubiquitylation substrates of STUB1, as exemplified herein.

The term “STUB1-regulated signaling pathway(s)” includes signaling pathways in which STUB1/CHIP (and its fragments, domains, and/or motifs thereof, discussed herein) binds to at least one of its substrate, through which at least one cellular function and/or activity and/or cellular protein profiles is changed (e.g., by promoting protein degradation of its substrates through the proteasome). In some embodiments, STUB1 promotes ubiquitylation and proteasome degradation for at least one of its substrates which bind to it. STUB1-regulated signaling pathways include at least those described herein, such as cellular response to misfoled proteins, DNA repaire, endoplasmic reticulum unfolded protein response, ERBB2 signaling pathway, misfolded or incompletely synthesized protein catabolic process, TGF-beta signaling pathway, chaperone-mediated protein complex assemply, ubiquitin-protein transferase activity, ubiquitin-depedent protein catabolic process, protein autoubiquitylation, protein maturation, glucocorticoid metabolic process, SMAD protein catabolic process, etc.

The term “STUB1 inhibitor(s)” includes any natural or non-natural agent prepared, synthesized, manufactured, and/or purified by human that is capable of reducing, inhibiting, blocking, preventing, and/or that inhibits the ability of a STUB1 polypeptide (and its fragments, domains, and/or motifs thereof, discussed herein). In one embodiment, such inhibitors may reduce or inhibit the binding/interaction between STUB1 and its substrates or other binding partners. In another embodiment, such inhibitors may reduce or inhibit the catalytic function of STUB1 as an E3 ubiquitylation ligase. In still another embodiment, such inhibitors may increase or promote the turnover rate, reduce or inhibit the expression and/or the stability (e.g., the half-life), and/or change the cellular localization of STUB1, resulting in at least a decrease in STUB1 levels and/or activity. Such inhibitors may be any molecule, including but not limited to small molecule compounds, antibodies or intrabodies, RNA interfereing (RNAi) agents (including at least siRNAs, shRNAs, microRNAs (miRNAs), piwi, and other well-known agents). Such inhibitors may be specific to STUB1 or also inhibit at least one of other E3 ubiquitin ligases. RNA interference for STUB1 polypeptides are well known and commercially available (e.g., human or mouse shRNA (Cat. # TR316909, TG316909, and TL503190) and siRNA (Cat. # SR306955 and SR408908) products and human or mouse gene knockout kit via CRISPR (Cat. # KN200310 and KN316920) from Origene (Rockville, Md.), siRNA/shRNA products (Cat. # sc-43555 and sc-44731) from Santa Cruz Biotechonology (Dallas, Tex.), etc.). Methods for detection, purification, and/or inhibition of STUB1 (e.g., by anti-STUB1 antibodies) are also well known and commercially available (e.g., multiple anti-STUB1 antibodies from Origene (Cat. # AP10553SU-N, AP15976PU-T, TA302767, TA322552, TA324315, TA324478, etc.), Cell Signaling Technology (Danvers, Mass., Cat. #2080), abcam (Cambridge, Mass., Cat. # ab2917, ab134064, ab109103, ab168768, ab180038, etc.), R&D Systems (Minneapolis, Minn., Cat. # AF4685), Santa Cruz Biotechnology (Cat. # sc-133066 and sc-133083), etc.). STUB1 knockout cell lines are also well known and commercially available (e.g., Cat # HZGHC10273 from Horizon Discovery, Canbridge, UK).

The term “UBQLN1,” also known as ubiquilin 1, refers to a member of a group of ubiquitin-like proteins (ubiquilin) that share a high degree of similarity with related products in yeast, rat and frog. Ubiquilins contain an N-terminal ubiquitin-like domain and a C-terminal ubiquitin-associated domain. They physically associate with both proteasomes and ubiquitin ligases, and thus are thought to functionally link the ubiquitination machinery to the proteasome to affect in vivo protein degradation. This ubiquilin has also been shown to modulate accumulation of presenilin proteins, and it is found in lesions associated with Alzheimer's and Parkinson's disease. Higher levels of ubiquilin-1 in the brain decreased malformation of the APP molecule which plays a key role in triggering Alzheimers disease, while lower levels of ubiquilin-1 in the brain were associated with increased malformation of APP (Stieren et al. (2011) J. Biol. Chem. 286:35689-35698). Two transcript variants encoding different isoforms have been found for this gene. UBQLN1 plays an important role in the regulation of different protein degradation mechanisms and pathways including ubiquitin-proteasome system (UPS), autophagy and endoplasmic reticulum-associated protein degradation (ERAD) pathway. UBQLN1 mediates the proteasomal targeting of misfolded or accumulated proteins for degradation by binding (via UBA domain) to their polyubiquitin chains and by interacting (via ubiquitin-like domain) with the subunits of the proteasome. UBQLN1 also plays a role in the ERAD pathway via its interaction with ER-localized proteins UBXN4, VCP and HERPUD1 and may form a link between the polyubiquitinated ERAD substrates and the proteasome. UBQLN1 plays a role in unfolded protein response (UPR) by attenuating the induction of UPR-inducible genes, DDTI3/CHOP, HSPA5 and PDIA2 during ER stress. UBQLN1 is involved in the regulation of macroautophagy and autophagosome formation and is required for maturation of autophagy-related protein LC3 from the cytosolic form LC3-I to the membrane-bound form LC3-II, in addition to asisting in the maturation of autophagosomes to autolysosomes by mediating autophagosome-lysosome fusion. UBQLN1 negatively regulates the TICAM1/TRIF-dependent toll-like receptor signaling pathway by decreasing the abundance of TICAM1 via the autophagic pathway. UBQLN1 accumulates to aggresomes, which may then be removed from cells by autophagocytosis. UBQLN1 promotes the ubiquitination and lysosomal degradation of ORAI1, consequently downregulating the ORAI1-mediated Ca²⁺ mobilization. UBQLN1 suppresses the maturation and proteasomal degradation of amyloid beta A4 protein (A4) by stimulating the lysine 63 (K63)-linked polyubiquitination. UBQLN1 delays the maturation of A4 by sequestering it in the Golgi apparatus and preventing its transport to the cell surface for subsequent processing. UBQLN1 is involved in pathways such as Clathrin-mediated endocytosis (e.g., cargo recognition process), vesicle-medated transport (e.g., membrane trafficking), protein processing in endoplasmic reticulum (ER), CXCR4-mediated signaling events, etc. UBQLN1 is suggested to bind to HERPUD1 (Kim et al. (2008) Biochem. Biophys. Res. Commun. 369:741-746), MTOR (Wu et al. (2002) Biochim. Biophys. Acta. 1542:41-56), P4HB (Ko et al. (2002) J. Biol. Chem. 277:35386-35392), PSEN1 and PSEN2 (Mah et al. (2000) J. Cell Biol. 151:847-862), UBE3A (Kleijnen et al. (2000) Mol. Cell. 6:409-419), TMCO6 (Rolland et al. (2014) Cell 159:1212-1226). UBQLN1 can form homodiers or heterordimers with UBQLN2. UBQLN1 is also suggested to bind CD47, NBL1, GABRA1, GABRA2, GABRA3, GABRA6, GABRB1, GABRB2, GABRB3, UBE3A, BTRC, P4HB, and MTOR. UBQLN1 interacts with the proteasome 19S subunit. UBQLN1 interacts (via ubiquitin-like domain) with TREX1 and may control TREX1 subcellular location. UBQLN1 forms a complex with UBXN4 and VCP. UBQLN1 also interacts (via UBA domain) with UBQLN4 (via ubiquitin-like domain). UBQLN1 is further found in a complex with UBQLN2 and MAP1LC3A/B/C. The monomeric form of UBQLN1 interacts with PSEN2 and PSEN1. UBQLN1 also interacts with ORAI1, TICAM1 (via the UBA domain of UBQLN1), EPS15, UBA52 (via the UBA domain of UBQLN1), PSMD3 and PSMD4 (via the ubiquitin-like domain of UBQLN1), HERPUD1, MAP1LC3A/B/C (in the presence of UBQLN4), EPS15 (via the ubiquitin-like domain of UBQLN1 and UIM domains of EPS15), EPS15L1 (via the ubiquitin-like domain of UBQLN1), HGS, and STAM2.

The nucleic acid and amino acid sequences of a representative human UBQLN1 is available to the public at the GenBank database (Gene ID 29979) and is shown in Table 1 (e.g., GenBank database numbers NM_013438.4 and NP_038466.2, representing the longer transcript variant 1 and the encoded longer protein isoform 1, and NM_053067.2 and NP_444295.1, representing the shorter transcript variant 2 (missing an alternate 3′ coding exon) and the encoded shorter protein isoform 2). The domain structure of UBQLN1 polypeptide is well known and accessible in UniProtKB database under the accession number Q9UMX0, including, in the order from the 5′ terminus to the 3′ terminus, a ubiquitin-like domin comprising, e.g., amino acid positions 37-111 of NP_038466.2, four STI1 domains comprising, e.g., amino acid positions 182-210, 212-251, 387-434, and 438-470 of NP_038466.2, and a UBA domain comprising, e.g., amino acid positions 546-586 of NP_038466.2. Dimerization of UBQIN1 is dependent upon the central region of the protein containing the STI1 domains and is independent of its ubiquitin-like and UBA domains (Ford and Monteiro (2006) Biochem. J. 399:397-404).

Nucleic acid and polypeptide sequences of UBQLN1 orthologs in organisms other than humans are well-known and include, for example, chimpanzee (Pan troglodytes) UBQLN1 (XM_009456778.2 and XP_009455053.2, and XM_016961026.1 and XP_016816515.1), dog UBQLN1 (XM_853048.4 and XP_858141.2, and XM_853126.4 and XP_858219.1), cattle UBQLN1 (NM_174628.1 and NP_777053.1), mouse UBQLN1 (NM_026842.4 and NP_081118.4, representing the longer transcript variant 1 and the encoded longer protein isoform 1, and NM_152234.2 and NP_689420.1, representing the shorter transcript variant 2 (lacking an in-frame exon in the 3′ coding region) and the encoded shorter protein isoform 2), Norway rat (Rattus norvegicus) UBQLN1 (NM_053747.2 and NP_446199.2), chicken UBQLN1 (XM_425028.4 and XP_425028.2, XM_004949193.2 and XP_004949250.1, and XM_004949194.2 and XP_004949251.1), tropical clawed frog UBQLN1 (NM_203890.1 and NP_989221.1), and fruit fly UBQLN1 (NM_134500.4 and NP_608344.1, and NM_001298528.1 and NP_001285457.1).

The term “UBQLN1 activity” includes the ability of a UBQLN1 polypeptide (and its fragments, domains, and/or motifs thereof, discussed herein) to bind its substrate and/or catalyze ubiquitination and proteosome degredation activity. UBQLN1 activity may also include one or more of functions, such as those disclosed herein in ubiquitin-proteasome pathway, unfolded protein response, Clathrin-mediated endocytosis, vesicle-mediated transport, and CXCR4-mediated signaling events. For example, UBQLN1 may interact with various proteins disclosed herein, such as its ubiquitination substrates and other binding partners, for it functions in signalings. UBQLN1 may also be proteolyticly modified, such as being glycosylated, or otherwise disclosed herein, for it functions.

The term “UBQLN1 substrate(s)” refers to binding partners of a UBQLN1 polypeptide (and its fragments, domains, and/or motifs thereof, discussed herein), e.g., the proteins listed herein. Furthermore, UBQLN1 substrates may refer to downstream members in the signaling pathways where UBQLN1 has a functional role, such as its ubiquitination substrates in the ubiquitin proteosome pathway.

The term “UBQLN1-regulated signaling pathway(s)” includes signaling pathways in which UBQLN1 (and its fragments, domains, and/or motifs thereof, discussed herein) binds to at least one of its substrate, through which at least one cellular function and/or activity and/or cellular protein profiles is changed (e.g., ubiquitin proteosome presentation). In some embodiments, UBQLN1 promotes unfolded protein response for at least one of its substrates which bind to it. UBQLN1-regulated signaling pathways include at least those described herein, such as ubiquitin-proteasome pathway, unfolded protein response, Clathrin-mediated endocytosis, vesicle-mediated transport, CXCR4-mediated signaling events, etc.

The term “UBQLN1 inhibitor(s)” includes any natural or non-natural agent prepared, synthesized, manufactured, and/or purified by human that is capable of reducing, inhibiting, blocking, preventing, and/or that inhibits the ability of a UBQLN1 polypeptide (and its fragments, domains, and/or motifs thereof, discussed herein). In one embodiment, such inhibitors may reduce or inhibit the binding/interaction between UBQLN1 and its substrates or other binding partners. In another embodiment, such inhibitors may reduce or inhibit UBQLN1 as a ubiquitin ligase for ubiquitin proteasome pathway or other related pathway described herein. In still another embodiment, such inhibitors may increase or promote the turnover rate, reduce or inhibit the expression and/or the stability (e.g., the half-life), and/or change the cellular localization of UBQLN1, resulting in at least a decrease in UBQLN1 levels and/or activity. Such inhibitors may be any molecule, including but not limited to small molecule compounds, antibodies or intrabodies, RNA interfereing (RNAi) agents (including at least siRNAs, shRNAs, microRNAs (miRNAs), piwi, and other well-known agents). Such inhibitors may be specific to UBQLN1 or also inhibit at least one of other ubiquitin ligases. RNA interference for UBQLN1 polypepitdes are well known and commercially available (e.g., human or mouse shRNA (Cat. # TL515928, TF300684, TF712115, etc.) products, siRNA products (Cat. # SR309322, SR417743, and SR502401), and human or mouse gene knockout kit via CRISPR (Cat. # KN318614 and KN204020) from Origene (Rockville, Md.), siRNA/shRNA products (Cat. # sc-41669 and sc-41670) and human or mouse gene knockout kit via CRISPR (Cat. # sc-425000) from Santa Cruz Biotechonology (Dallas, Tex.), and Ready-to-package AAV shRNA clones from Vigene Biosciences (Rockville, Md., Cat. # SH801808 and SH886096), etc.). Methods for detection, purification, and/or inhibition of UBQLN1 (e.g., by anti-UBQLN1 antibodies) are also well known and commercially available (e.g., multiple anti-UBQLN1 antibodies from Origene (Cat. # TA302118, TA302119, TA308768, TA324714, TA334488, TA349560, and others), Novus Biologicals (Littleton, Colo., Cat. # NBP1-56536, AF7969, H00029979-M01, H00029979-M02, etc.), abcam (Cambridge, Mass., Cat. # ab3314 and ab128011, etc.), etc.). UBQLN1 knockout human cell lines are also well known and available at the Horizon (Cambridge, UK, Cat. # HZGHC004088c002).

The term “HSP90B1,” also known as heat shock protein 90 kDa beta member 1, endoplasmin, gp96, grp94, and ERp99, refers to a member of a family of adenosine triphosphate(ATP)-metabolizing molecular chaperones with roles in stabilizing and folding other proteins, as well as the processing and transport of secreted proteins. The encoded protein HSP90B1 is localized to melanosomes and the endoplasmic reticulum. Expression of this protein is associated with a variety of pathogenic states, including tumor formation. There is a microRNA gene located within the 5′ exon of this gene. There are pseudogenes for this gene on chromosomes 1 and 15. HSP90B1 plays critical roles in folding proteins in the secretory pathway such as Toll-like receptors and integrins (Randow and Seed (2001) Nat. Cell Biol. 3:891-896; Yang et al. (2007) Immunity 26:215-226). HSP90B1 has been implicated as an essential immune chaperone to regulate both innate and adaptive immunity (Schild and Rammensee (2000) Nat. Immunol. 1:100-101). Tumor-derived HSP90B1 (vitespen) has entered clinical trials for cancer immunotherapy. HSP90B1 has ATPase activity and functions in ER-associated degradation (ERAD) and, when associated with CNPY3, is required for proper folding of Toll-like receptors. HSP90B1 is involved in pathways such as Unfolded Protein Response (UPR) (e.g., ATF6-alpha activated chaperones), estrogen signaling pathway (e.g., in glioma and/or prostate cancer), binding and uptake of ligands by scavenger receptors, cytokins signaling in immune system, actin dynamics signaling pathway, AKT signaling pathway, glucose/energy metabolism, IL6-mediated signaling events, SIDS susceptibility pathways, etc. HSP90B1 is suggested to bind to FANCC, CDC5L, CSNK2A1, POLR2E, SNW1, etc. HSP90B1 can form homodimers through disulfide-linkage. HSP90B1 is also a component of an EIF2 complex at least composed of CELF1/CUGBP1, CALR, CALR3, EIF2S1, EIF2S2, HSP90B1 and HSPA5. HSP90B1 is part of a large chaperone multiprotein complex comprising DNAJB11, HSP90B1, HSPA5, HYOU, PDIA2, PDIA4, PDIA6, PPIB, SDF2L1, UGT1A1 and very small amounts of ERP29, but not, or at very low levels, CALR nor CANX. HSP90B1 interacts with AIMP1 and regulates its retention in the endoplasmic reticulum. Similarly, HSP90B1 interacts with OS9, CNPY3, TLR4, METTL23, and TLR9, but not with TLR3. HSP90B1 also interacts with MZB 1 in a calcium-dependent manner. HSP90B1 is related to at least oral cavity cancer, bipolar disorder, and prostate cancer.

The nucleic acid and amino acid sequences of a representative human HSP90B1 is available to the public at the GenBank database (Gene ID 7184) and is shown in Table 1 (e.g., GenBank database numbers NM_003299.2 and NP_003290.1). The domain structure of HSP90B1 polypeptide is well known and accessible in UniProtKB database under the accession number P14625, including, in the order from the 5′ terminus to the 3′ terminus, a signal peptide comprising, e.g., amino acid positions 1-21 of NP_003290.1, a histidine kinase-like ATPase (HATPase) domain comprising, e.g., amino acid positions 96-254 of NP_003290.1, an HSP90 domain comprising, e.g., amino acid positions 257-755 of NP_003290.1, and an ER retention motif comprising, e.g., amino acid positions 800-803 of NP 003290.1.

Nucleic acid and polypeptide sequences of HSP90B1 orthologs in organisms other than humans are well-known and include, for example, chimpanzee (Pan troglodytes) HSP90B1 (NM_001246487.1 and NP_001233416.1), Rhesus monkey HSP90B1 (NM_001195524.1 and NP_001182453.1), dog HSP90B1 (NM_001003327.2 and NP_001003327.1), cattle HSP90B1 (NM_174700.2 and NP_777125.1), mouse HSP90B1 (NM_011631.1 and NP_035761.1), Norway rat (Rattus norvegicus) HSP90B1 (NM_001012197.2 and NP_001012197.2), chicken HSP90B1 (NM_204289.1 and NP_989620.1), tropical clawed frog HSP90B1 (NM_001045763.1 and NP_001039228.1), and zebrafish HSP90B1 (NM_198210.2 and NP_937853.1).

The term “HSP90B1 activity” includes the ability of an HSP90B1 polypeptide (and its fragments, domains, and/or motifs thereof, discussed herein) to bind its substrate and/or catalyze its ATPase and chaperon activity. HSP90B1 activity may also include one or more of functions, such as those disclosed herein in ER-associated degradation, Unfolded Protein Response (UPR) (e.g., ATF6-alpha activated chaperones), estrogen signaling pathway (e.g., in glioma and/or prostate cancer), binding and uptake of ligands by scavenger receptors, cytokins signaling in immune system, actin dynamics signaling pathway, AKT signaling pathway, glucose/energy metabolism, IL6-mediated signaling events, and SIDS susceptibility pathways. For example, HSP90B1 may interact with various proteins disclosed herein, such as its ATPase and/or chaperon substrates and other binding partners, for it functions in signalings. HSP90B1 may also be proteolyticly modified, such as being glycosylated, or otherwise disclosed herein, for it funcations.

The term “HSP90B1 substrate(s)” refers to binding partners of an HSP90B1 polypeptide (and its fragments, domains, and/or motifs thereof, discussed herein), e.g., the proteins listed herein. Furthermore, HSP90B1 substrates may refer to downstream members in the signaling pathways where HSP90B1 has a functional role, such as its ATPase and/or chaperon substrates or other substrates in ER-associated degradation and/or other related signaling pathways as described herein.

The term “HSP90B1-regulated signaling pathway(s)” includes signaling pathways in which HSP90B1 (and its fragments, domains, and/or motifs thereof, discussed herein) binds to at least one of its substrate, through which at least one cellular function and/or activity and/or cellular protein profiles is changed (e.g., unfolded protein response). In some embodiments, HSP90B1 functions as a molecular chaperon for at least one of its substrates which bind to it. HSP90B1-regulated signaling pathways include at least those described herein, such as ER-associated degradation, Unfolded Protein Response (UPR) (e.g., ATF6-alpha activated chaperones), estrogen signaling pathway (e.g., in glioma and/or prostate cancer), binding and uptake of ligands by scavenger receptors, cytokins signaling in immune system, actin dynamics signaling pathway, AKT signaling pathway, glucose/energy metabolism, IL6-mediated signaling events, SIDS susceptibility pathways, etc.

The term “HSP90B1 inhibitor(s)” includes any natural or non-natural agent prepared, synthesized, manufactured, and/or purified by human that is capable of reducing, inhibiting, blocking, preventing, and/or that inhibits the ability of an HSP90B1 polypeptide (and its fragments, domains, and/or motifs thereof, discussed herein). In one embodiment, such inhibitors may reduce or inhibit the binding/interaction between HSP90B1 and its substrates or other binding partners. In another embodiment, such inhibitors may reduce or inhibit HSP90B1 as an ATPase and a chaperon for unfoled protein response or other related pathway described herein. In still another embodiment, such inhibitors may increase or promote the turnover rate, reduce or inhibit the expression and/or the stability (e.g., the half-life), and/or change the cellular localization of HSP90B1, resulting in at least a decrease in HSP90B1 levels and/or activity. Such inhibitors may be any molecule, including but not limited to small molecule compounds, antibodies or intrabodies, RNA interfereing (RNAi) agents (including at least siRNAs, shRNAs, microRNAs (miRNAs), piwi, and other well-known agents). Such inhibitors may be specific to HSP90B1 or also inhibit at least one of other ATPase and/or chaperons. Some exemplary inhibitors include, at least, Geldanamycin (1,4-benzoquinone ansamycin), Radicicol, Diglyme (1-Methoxy-2-(2-Methoxyethoxy)Ethane), etc. Some commercially available inhibitors at APExBio (Houston, Tex.) include, at least, AT13387 (Cat. # A4056), 17-DMAG (Alvespimycin) HCl (Cat. # A2213), XL-888 (Cat. # A4388), PF-04929113 (SNX-5422) (Cat. # A4065), NVP-BEP800 (Cat. # A4064), MPC-3100 (Cat. # A4063), IPI-504 (Retaspimycin hydrochloride) (Cat. # A4061), BIIB021 (Cat. # A44058), EC 144 (Cat. # B4920), HSP990 (NVP-HSP990) (Cat. # B4797), Gedunin (Cat. # B7393), Macbecin I (Cat. # B7259), 17-AAG Hydrochloride (Cat. # A3109), 17-AAG (KOS953) (Cat. # A4054), KW-2478 (Cat. # A4062), AUY922 (NVP-AUY922) (Cat. # A4057), KW-2478 (Cat. # A4054), Ganetespib (STA-9090) (Cat. # A4385), Alvespimycin (Cat. # A3161), PU-H71 (Cat. # A3739), Retaspimycin (Cat. # A3756), and SNX-2112 (Cat. # A4068). RNA interference for HSP90B1 polypepitdes are well known and commercially available (e.g., human or mouse shRNA (Cat. # TG312309, TF511402, TF701702, etc.) products, siRNA products (Cat. # SR304925, SR420277, and SR505295), and human or mouse gene knockout kit via CRISPR (Cat. # KN307995 and KN209563) from Origene (Rockville, Md.), siRNA/shRNA products (Cat. # sc-35523, sc-44304, and sc-35524) and human or mouse gene knockout kit via CRISPR (Cat. # sc-400605) from Santa Cruz Biotechonology (Dallas, Tex.), and Ready-to-package AAV shRNA clones from Vigene Biosciences (Rockville, Md., Cat. # SH829080), etc.). Methods for detection, purification, and/or inhibition of HSP90B1 (e.g., by anti-HSP90B1 antibodies) are also well known and commercially available (e.g., multiple anti-HSP90B1 antibodies from Origene (Cat. #TA324795, TA322868, AM09037PU-N, AM50614PU-N, and others), Novus Biologicals (Littleton, Colo., Cat. # NB300-619, MAB7606, NBP2-42379, NBP2-44690, etc.), abcam (Cambridge, Mass., Cat. # ab2791, ab3674, ab212912, ab210960, ab212034, etc.), Santa Cruz Biotechnology (Cat. # sc-32249, sc-1794, sc-11402, and sc-56399), etc.).

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “interaction”, when referring to an interaction between two molecules, refers to the physical contact (e.g., binding) of the molecules with one another. Generally, such an interaction results in an activity (which produces a biological effect) of one or both of said molecules.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM, IgG1, IgG2C, and the like) that is encoded by heavy chain constant region genes.

As used herein, the term “KD” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

A “kit” is any manufacture (e.g. a package or container) comprising at least one reagent, e.g. a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

The term “neoadjuvant therapy” refers to a treatment given before the primary treatment. Examples of neoadjuvant therapy can include chemotherapy, radiation therapy, and hormone therapy. For example, in treating breast cancer, neoadjuvant therapy can allows patients with large breast cancer to undergo breast-conserving surgery.

The “normal” level of expression of a biomarker is the level of expression of the biomarker in cells of a subject, e.g., a human patient, not afflicted with a cancer. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “pre-determined” biomarker amount and/or activity measurement(s) may be a biomarker amount and/or activity measurement(s) used to, by way of example only, evaluate a subject that may be selected for a particular treatment, evaluate a response to a treatment such as combination therapies with an immunotherapy and an inhibitor of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and/or evaluate the disease state. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a cancer to combination therapies with an immunotherapy (e.g., using an immune checkpoint inhibitor) and an inhibitor of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)). Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer (e.g., those responding to a particular inhibitor of one or more biomarkers listed in Table 1/immunotherapy combination therapy or those developing resistance thereto).

The term “pre-malignant lesions” as described herein refers to a lesion that, while not cancerous, has potential for becoming cancerous. It also includes the term “pre-malignant disorders” or “potentially malignant disorders.” In particular this refers to a benign, morphologically and/or histologically altered tissue that has a greater than normal risk of malignant transformation, and a disease or a patient's habit that does not necessarily alter the clinical appearance of local tissue but is associated with a greater than normal risk of precancerous lesion or cancer development in that tissue (leukoplakia, erythroplakia, erytroleukoplakia lichen planus (lichenoid reaction) and any lesion or an area which histological examination showed atypia of cells or dysplasia. In one embodiment, a metaplasia is a pre-malignant lesion.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., solid tumors, such as esophageal cancer and gastric cancer), development of one or more clinical factors, or recovery from the disease.

The term “response to immunotherapy” or “response to inhibitor of one or more biomarkers/immunotherapy combination therapy” relates to any response of the hyperproliferative disorder (e.g., cancer) to an anti-cancer agent, such as an inhibitor of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), with or without an immunotherapy, preferably to a change in tumor mass and/or volume after initiation of neoadjuvant or adjuvant therapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of“body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, may be administered to a patient having or at risk for having cancer, to inhibit expression of a biomarker gene which is overexpressed in cancer and thereby treat, prevent, or inhibit cancer in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (KD) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a cancer, e.g., brain, lung, ovarian, pancreatic, liver, breast, prostate, and/or colorectal cancers, melanoma, multiple myeloma, and the like. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “synergistic effect” refers to the combined effect of two or more anti-cancer agents (e.g., inhibitors of one or more biomarkers listed in Table 1/immunotherapy combination therapy) can be greater than the sum of the separate effects of the anti-cancer agents/therapies alone.

The term “T cell” includes CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

As used herein, the term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134).

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers of the present invention (e.g., biomarkers listed in Tables 1 and 2) are well-known in the art and readily available on publicly available databases, such as the National Center for Biotechnology Information (NCBI). For example, exemplary nucleic acid and amino acid sequences derived from publicly available sequence databases are provided below and include, for example, PCT Publ. WO 2014/022759, which is incorporated herein in its entirety by this reference.

TABLE 1
SEQ ID NO: 1 Human STUB1 Variant 1 cDNA Sequence (NM_005861.3,
CDS region from position 416-1327)

| | |
|---|---|
| 1 | acaggctgtt gcgggagcgc gccctcagcg aagcaagtga ggcatctcac tgggaaagtc |
| 61 | gaatgtgtgt ggcggccgcc gccgaggcgg gttccgaaga gacctcagca gggcaggcca |
| 121 | gggcctacgc gaacgcccac ccttaagagc gcggggacag ggaactggag cgttcctccc |
| 181 | agcccccgac gtcgcgggcc cagtgtcccc gtccaggctg gttgggcgca cgcgcggccc |
| 241 | cactcgcccc cacgcgtgcg tccccgctgg tcccgccccc ggccggaagt tccggcggcg |
| 301 | gagctgggcc gggcccgagc ggatcgcggg ctcgggctgc ggggctccgg ctgcgggcgc |
| 361 | tgggccgcga ggcgcggagc ttgggagcgg agcccaggcc gtgccgcgcg gcgccatgaa |
| 421 | gggcaaggag gagaaggagg gcggcgcacg gctgggcgct ggcggcggaa gccccgagaa |
| 481 | gagcccgagc gcgcaggagc tcaaggagca gggcaatcgt ctgttcgtgg gccgaaagta |
| 541 | cccggaggcg gcggcctgct acggccgcgc gatcacccgg aacccgctgg tggccgtgta |
| 601 | ttacaccaac cgggccttgt gctacctgaa gatgcagcag cacgagcagg ccctggccga |
| 661 | ctgccggcgc gccctggagc tggacgggca gtctgtgaag gcgcacttct tcctggggca |
| 721 | gtgccagctg gagatggaga gctatgatga ggccatcgcc aatctgcagc gagcttacag |
| 781 | cctggccaag gagcagcggc tgaacttcgg ggacgacatc cccagcgctc ttcgaatcgc |
| 841 | gaagaagaag cgctggaaca gcattgagga gcggcgcatc caccaggaga gcgagctgca |
| 901 | ctcctacctc tccaggctca ttgccgcgga gcgtgagagg gagctggaag agtgccagcg |
| 961 | aaaccacgag ggtgatgagg acgacagcca cgtccgggcc cagcaggcct gcattgaggc |
| 1021 | caagcacgac aagtacatgg cggacatgga cgagcttttt tctcaggtgg atgagaagag |
| 1081 | gaagaagcga gacatccccg actacctgtg tggcaagatc agctttgagc tgatgcggga |
| 1141 | gccgtgcatc acgcccagtg gcatcaccta cgaccgcaag gacatcgagg agcacctgca |
| 1201 | gcgtgtgggt cattttgacc ccgtgacccg gagccccctg acccaggaac agctcatccc |
| 1261 | caacttggct atgaaggagg ttattgacgc attcatctct gagaatggct gggtggagga |
| 1321 | ctactgaggt tccctgccct acctggcgtc ctggtccagg ggagccctgg gcagaagccc |
| 1381 | ccggccccta tacatagttt atgttcctgg ccaccccgac cgcttccccc aagttctgct |
| 1441 | gttggactct ggactgtttc ccctctcagc atcgcttttg ctgggccgtg atcgtccccc |
| 1501 | tttgtgggct ggaaaagcag gtgagggtgg gctgggctga ggccattgcc gccactatct |
| 1561 | gtgtaataaa atccgtgagc acgaggtggg acgtgctggt gtgtgaaaaa aaaaaaaaaa |
| 1621 | aaaaaaaaaa aaaaaaaaaa aaaaaaaaaa |

SEQ ID NO: 2 Human STUB1 isoform a Amino Acid Sequence (NP_005852.2)

| | |
|---|---|
| 1 | mkgkeekegg arlgagggsp ekspsagelk eggnrlfvgr kypeaaacyg raitrnplva |
| 61 | vyytnralcy lkmgghegal adcrraleld gqsvkahffl gqcqlemesy deaianlgra |
| 121 | yslakegrln fgddipsalr iakkkrwnsi eerrihgese lhsylsrlia aerereleec |
| 181 | qrnhegdedd shvraqqaci eakhdkymad mdelfsqvde krkkrdipdy lcgkisfelm |
| 241 | repcitpsgi tydrkdieeh lqrvghfdpv trspltqeql ipnlamkevi dafisengwv |
| 301 | edy |

SEQ ID NO: 3 Human STUB1 Variant 2 cDNA Sequence (NM_001293197.1,
CDS region from position 721-1416)

| | |
|---|---|
| 1 | acaggctgtt gcgggagcgc gccctcagcg aagcaagtga ggcatctcac tgggaaagtc |
| 61 | gaatgtgtgt ggcggccgcc gccgaggcgg gttccgaaga gacctcagca gggcaggcca |
| 121 | gggcctacgc gaacgcccac ccttaagagc gcggggacag ggaactggag cgttcctccc |
| 181 | agcccccgac gtcgcgggcc cagtgtcccc gtccaggctg gttgggcgca cgcgcggccc |
| 241 | cactcgcccc cacgcgtgcg tccccgctgg tcccgccccc ggccggaagt tccggcggcg |
| 301 | gagctgggcc gggcccgagc ggatcgcggg ctcgggctgc ggggctccgg ctgcgggcgc |
| 361 | tgggccgcga ggcgcggagc ttgggagcgg agcccaggcc gtgccgcgcg gcgccatgaa |
| 421 | gggcaaggag gagaaggagg gcggcgcacg gctgggcgct ggcggcggaa gccccgagaa |
| 481 | gagcccgagc gcgcaggagc tcaaggagca gggcaatcgt ctgttcgtgg gccgaaagta |
| 541 | cccggaggcg gcggcctgct acggccgcgc gatcccagag cgcagaagct gggacgggcc |
| 601 | gtgggtcaga gtgggcacgc tgagcctacg ccctcatgcg gctggcccgg ccttggtccc |
| 661 | tagacccgga acccgctggt ggccgtgtat tacaccaacc gggccttgtg ctacctgaag |
| 721 | atgcagcagc acgagcaggc cctggccgac tgccggcgcg ccctggagct ggacgggcag |
| 781 | tctgtgaagg cgcacttctt cctggggcag tgccagctgg agatggagag ctatgatgag |
| 841 | gccatcgcca atctgcagcg agcttacagc ctggccaagg agcagcggct gaacttcggg |
| 901 | gacgacatcc ccagcgctct tcgaatcgcg aagaagaagc gctggaacag cattgaggag |
| 961 | cggcgcatcc accaggagag cgagctgcac tcctacctct ccaggctcat tgccgcggag |
| 1021 | cgtgagaggg agctggaaga gtgccagcga aaccacgagg gtgatgagga cgacagccac |
| 1081 | gtccgggccc agcaggcctg cattgaggcc aagcacgaca agtacatggc ggacatggac |
| 1141 | gagctttttt ctcaggtgga tgagaagagg aagaagcgag acatccccga ctacctgtgt |
| 1201 | ggcaagatca gctttgagct gatgcgggag ccgtgcatca cgcccagtgg catcacctac |
| 1261 | gaccgcaagg acatcgagga gcacctgcag cgtgtgggtc attttgaccc cgtgacccgg |
| 1321 | agccccctga cccaggaaca gctcatcccc aacttggcta tgaaggaggt tattgacgca |
| 1381 | ttcatctctg agaatggctg ggtggaggac tactgaggtt ccctgcccta cctggcgtcc |
| 1441 | tggtccaggg gagccctggg cagaagcccc cggcccctat acatagttta tgttcctggc |
| 1501 | caccccgacc gcttccccca agttctgctg ttggactctg gactgtttcc cctctcagca |
| 1561 | tcgcttttgc tgggccgtga tcgtccccct ttgtgggctg gaaaagcagg tgagggtggg |
| 1621 | ctgggctgag gccattgccg ccactatctg tgtaataaaa tccgtgagca cgaggtggga |
| 1681 | cgtgctggtg tgtgaaaaaa aaaaaaaaaa aaaaaaaaaa aaaaaaaaaa aaaaaaaaa |

SEQ ID NO: 4 Human STUB1 isoform b Amino Acid Sequence (NP_001280126.1)

| | |
|---|---|
| 1 | mqqheqalad crraleldgq svkahfflgq cqlemesyde aianlqrays lakeqrinfg |
| 61 | ddipsalria kkkrwnsiee rrihqeselh sylsrliaae rereleecqr nhegdeddsh |
| 121 | vraqqaciea khdkymadmd elfsqvdekr kkrdipdylc gkisfelmre pcitpsgity |
| 181 | drkdieehlq rvghfdpvtr spltqeqlip nlamkevida fisengwved y |

SEQ ID NO: 5 Mouse STUB1 cDNA Sequence (NM_019719.3, CDS region
from position 509-1423)

| | |
|---|---|
| 1 | ggccgcaggc agctcctccc gattcccgag gaagcccctc gagagcggtc tgatctgcgg |
| 61 | gcctctcttc taccctcaat tccgccttcc taccggcggt ccagacgttc ccaggctgtc |
| 121 | gcgtgagccc gtgtgtgccc tggtggtgcg ctaggacccg ggcctagtga ccggccctcg |
| 181 | gaaggccact tctcaatgac cgctgccacg ttcgcccagc ttggggccgg agggcggggc |
| 241 | ttgagcgccg atgggcggag ccttggtctg agcagggtag gtctcggtta ctgtagggcg |
| 301 | gggcctgtag agcgcggcgg aagcttctca gagctaaggg aactggctca atccacgagg |
| 361 | ctccgccttt gctcacgtgt ccccatgcca gctccgcccc acaccggaag ttccggtggc |
| 421 | ggatcgccga ccgggcggag ctgatcgctg cgcgggctgc gagatctagg tggccgggcg |
| 481 | cggagcccaa gccgtgccgc gcggcgccat gaagggcaag gaggaaaagg agggcggcgc |
| 541 | gcggctgggc actggtggcg gcggcagccc tgataagagc ccgagtgcgc aagagctcaa |
| 601 | ggagcaggga aaccggctct tcgtgggccg caagtacccg gaggcggcgg cctgctacgg |
| 661 | ccgcgccatc actcggaacc cacttgtggc agtgtactac actaaccggg ccctgtgcta |
| 721 | tctgaagatg cagcagcctg aacaggcact tgctgactgc cggcgagccc tggagctgga |
| 781 | cgggcagtct gtgaaggcgc acttcttcct ggggcagtgc cagctggaga tggagagtta |
| 841 | tgatgaggcc attgccaatc tgcagcgagc ctatagtttg gccaaggagc agcgactcaa |
| 901 | ctttggggat gatattccta gtgcccttcg cattgctaag aagaagcgct ggaacagtat |
| 961 | cgaggaacgg cgcatccacc aggagagtga gctgcattca tatctcacca ggctcattgc |
| 1021 | tgctgagcga gagagggaac tggaggagtg tcagcggaac cacgagggtc atgaagatga |
| 1081 | tggccacatc cgggcccagc aggcctgcat tgaggccaag cacgataaat acatggcaga |
| 1141 | tatggatgag ctcttctctc aggtggacga gaaaagaaag aagcgagata tccctgacta |
| 1201 | cttgtgtggc aagattagct ttgagctgat gcgggaaccc tgcattacac ccagtggtat |
| 1261 | cacctatgac cgcaaggaca ttgaggagca cctgcagcgt gtgggccact ttgaccctgt |
| 1321 | gacccggagc cctctgaccc aggaacagct catccccaac ttggccatga aggaagtcat |
| 1381 | tgacgctttc atctctgaga acggctgggt agaggactat tgaggcccca tgtcctgcct |
| 1441 | ggcaccctgg cccaggagga tctggagacg gaagctccag tccctgtata gtttgtgtcc |
| 1501 | ctgggcctgc ccccatcggc cctgctgatg ggttctgaac tgctcccctt ctcagcatac |
| 1561 | cccttgctgg accatgagcc tcccttgtcc cccttctggg ctggagagtg ggtgagggtg |
| 1621 | ggctgaggtt gctgctgctg ccactgtcct gtaataaagt ctgtgagcac ta |

SEQ ID NO: 6 Mouse STUB1 Amino Acid Sequence (NP_062693.1)

| | |
|---|---|
| 1 | mkgkeekegg arlgtggggs pdkspsaqel keqgnrlfvg rkypeaaacy graitrnplv |
| 61 | avyytnralc ylkmqqpeqa ladcrralel dgqsvkahff lgqcqlemes ydeaianlqr |
| 121 | ayslakeqrl nfgddipsal riakkkrwns ieerrihqes elhsyltrli aaererelee |
| 181 | cqrnheghed dghiraqqac leakhdkyma dmdelfsqvd ekrkkrdipd ylcgkisfel |
| 241 | mrepcitpsg itydrkdiee hlqrvghfdp vtrspltqeq lipnlamkev idafisengw |
| 301 | vedy |

SEQ ID NO: 7 Human UBQLN1 Variant 1 cDNA Sequence (NM_013438.4,
CDS region from position 575-2344)

| | |
|---|---|
| 1 | aatttcgcta accgtgacgt catctcggtg cgccgttaca gcgcttaact ggaaactgcg |
| 61 | atttagtttt tggtgaaagg agtggaaaaa aaccatgaac tgacctaccc cgccccattt |
| 121 | tgcttttagt ccttcacact ggcttcgcct gcactttggg caatggagcg ggaaaggaaa |
| 181 | aggcacgcag gctgagctca aagtcgcgcc agccggggat gactcggcga cgccgccagg |
| 241 | cgcgttaccc ggcgtgctcc gcgcggcgcc ggcgaaggga cgtgggggaa ggggcaggga |
| 301 | ggaggaagcg gtggctgctg cggatgtcgg tgtgagcgag cggcgcctga acacacggcg |
| 361 | gctgccgagc gcctgacccg ggcctgcgcc agagcctgca ccgagctccg gggccccaca |
| 421 | cccgctacgg tggccctgcg cccgttgcta ctgaggcggc gtgctctgca ttcttcgctg |
| 481 | tccaggcctg ccggctctgg tgtctgctgg ctcctccttg ctcgcctgct ccctcctgct |
| 541 | tgcctgagtc accgccgccg ccgccgccac agccatggcc gagagtggtg aaagcggcgg |
| 601 | tcctccgggc tcccaggata gcgccgccgg agccgaaggt gctggcgccc ccgcggccgc |
| 661 | tgcctccgcg gagcccaaaa tcatgaaagt caccgtgaag accccgaagg aaaaggagga |
| 721 | attcgccgtg cccgagaata gctccgtcca gcagtttaag gaagaaatct ctaaacgttt |
| 781 | taaatcacat actgaccaac ttgtgttgat atttgctgga aaaattttga aagatcaaga |
| 841 | taccttgagt cagcatggaa ttcatgatgg acttactgtt caccttgtca ttaaaacaca |
| 901 | aaacaggcct caggatcatt cagctcagca aacaaataca gctggaagca atgttactac |
| 961 | atcatcaact cctaatagta actctacatc tggttctgct actagcaacc cttttggttt |
| 1021 | aggtggcctt gggggacttg caggtctgag tagcttgggt ttgaatacta ccaacttctc |
| 1081 | tgaactacag agtcagatgc agcgacaact tttgtctaac cctgaaatga tggtccagat |
| 1141 | catggaaaat ccctttgttc agagcatgct ctcaaatcct gacctgatga gacagttaat |
| 1201 | tatggccaat ccacaaatgc agcagttgat acagagaaat ccagaaatta gtcatatgtt |
| 1261 | gaataatcca gatataatga gacaaacgtt ggaacttgcc aggaatccag caatgatgca |
| 1321 | ggagatgatg aggaaccagg accgagcttt gagcaaccta gaaagcatcc cagggggata |
| 1381 | taatgcttta aggcgcatgt acacagatat tcaggaacca atgctgagtg ctgcacaaga |
| 1441 | gcagtttggt ggtaatccat ttgcttcctt ggtgagcaat acatcctctg gtgaaggtag |
| 1501 | tcaaccttcc cgtacagaaa atagagatcc actacccaat ccatgggctc cacagacttc |
| 1561 | ccagagttca tcagcttcca gcggcactgc cagcactgtg ggtggcacta ctggtagtac |
| 1621 | tgccagtggc acttctgggc agagtactac tgcgccaaat ttggtgcctg gagtaggagc |
| 1681 | tagtatgttc aacacaccag gaatgcagag cttgttgcaa caaataactg aaaacccaca |
| 1741 | actgatgcaa aacatgttgt ctgcccccta catgagaagc atgatgcagt cactaagcca |
| 1801 | gaatcctgac cttgctgcac agatgatgct gaataatccc ctatttgctg gaaatcctca |
| 1861 | gcttcaagaa caaatgagac aacagctccc aactttcctc caacaaatgc agaatcctga |
| 1921 | tacactatca gcaatgtcaa accctagagc aatgcaggcc ttgttacaga ttcagcaggg |
| 1981 | tttacagaca ttagcaacgg aagccccggg cctcatccca gggtttactc ctggcttggg |
| 2041 | ggcattagga agcactggag gctcttcggg aactaatgga tctaacgcca cacctagtga |
| 2101 | aaacacaagt cccacagcag gaaccactga acctggacat cagcagttta ttcagcagat |
| 2161 | gctgcaggct cttgctggag taaatcctca gctacagaat ccagaagtca gatttcagca |
| 2221 | acaactggaa caactcagtg caatgggatt tttgaaccgt gaagcaaact tgcaagctct |
| 2281 | aatagcaaca ggaggtgata tcaatgcagc tattgaaagg ttactgggct cccagccatc |
| 2341 | atagcagcat ttctgtatct tgaaaaaatg taatttattt ttgataacgg ctcttaaact |
| 2401 | ttaaaatacc tgctttattt cattttgact cttggaattc tgtgctgtta taaacaaacc |
| 2461 | caatatgatg cattttaagg tggagtacag taagatgtgt gggtttttct gtatttttct |
| 2521 | tttctggaac agtgggaatt aaggctactg catgcatcac ttctgcattt attgtaattt |
| 2581 | tttaaaaaca tcacctttta tagttgggtg accagatttt gtcctgcatc tgtccagttt |
| 2641 | atttgctttt taaacattag cctatggtag taatttatgt agaataaaag cattaaaaag |
| 2701 | aagcaaatca tttgcactct ataatttgtg gtacagtatt gcttattgtg actttggcat |
| 2761 | gcatttttgc aaacaatgct gtaagattta tactactgat aattttgttt tatttgtata |
| 2821 | caatatagag tatgcacatt tgggactgca tttctggaaa catactgcaa taggctctct |
| 2881 | gagcaaaaca cctgtaacta aaaaagtgaa gataagaaaa tactcttaaa gctgagtatt |
| 2941 | tcctaattgt atagaatctt acagcatctt tgacaaacat ctcccagcaa aagtgccggt |
| 3001 | tagtcaggtt tgttgaaaat acagtagaaa agctgattct ggttatctct ttaaggacaa |
| 3061 | ttaattgtac agacacataa tgtaacattg tctcaacatt cattcacaga ttgactgtaa |
| 3121 | attaccttaa tctttgtgca gactgaagga acactgtagt ataccccaaa gtgcatttgc |
| 3181 | ctaggacttc tcagcttctc ccataggtag tttaacaggc attaaaattt gtaattgaaa |
| 3241 | tgttgctttc actgaaaaag tgtcttgatg tttcagttat ttttaatcgc cataaaaaaa |
| 3301 | tagaactatc ttttgggttt atctgttttc tcatgcacag gcaatacaca aatttaaaat |
| 3361 | gagttgtgag ccaattgttt ctgaagtgtt ttggtagttc tattaagaaa tagttaaata |
| 3421 | ttgtgctttt cagagcctca gagaaagggg gacggggtgg gggggtgggg cagcggaatc |
| 3481 | tgtcctggat ggggccagct taaataatac tggcaaccaa gattctgtta ggatttctgt |
| 3541 | gcatatagtg tagtaaagaa gtatcattca ggggtgaaaa acaaagagcc gttttaatga |
| 3601 | tgttgagtac atttggctgt tttatagcct ttttcttccc tcccccaaag aattctgttt |
| 3661 | gcctaactcc caaactgttg gggtggtaca ttcctttagg accaattaaa acataattga |
| 3721 | gggtcagtga tacatttggc tgactctggt tcagtattct cttaggtgat tatattctct |
| 3781 | catgtacagt tacaggaaat taaaatgtta aagtaaccta aaatgaattc agaccaataa |
| 3841 | aatcaaggga aatacaagtt gattgcatta cttctgtatg ttgcttgcta ttaaaaaggt |
| 3901 | taagaggcca ggttacccac cagtccttgc actgttctga cactttcccc aggaggaaaa |
| 3961 | caagtacaaa ggttacggtg gaggcataag tagaagagat tgttaagaag ggtattcatg |
| 4021 | tgtctttgct ctttctgctt tatgcctcag tttggtttaa aaacttctgt actggcaaat |
| 4081 | ggtggtattc agtgtgggat agtgtcataa ctaatttgac aatttattaa tcataaaata |
| 4141 | acaataaatc tctagctttt acacttgaaa aaaaaaa |

SEQ ID NO: 8 Human UBQLN1 Isoform 1 Amino Acid Sequence (NP_038466.2)

| | |
|---|---|
| 1 | maesgesggp pgsqdsaaga egagapaaaa saepkimkvt vktpkekeef avpenssvqq |
| 61 | fkeeiskrfk shtdqlvlif agkilkdqdt lsqhgihdgl tvhlviktqn rpqdhsaqqt |
| 121 | ntagsnvtts stpnsnstsg satsnpfglg glgglaglss lglnttnfse lqsqmqrqll |
| 181 | snpemmvqim enpfvqsmls npdlmrqlim anpqmqqliq rnpeishmln npdimrqtle |
| 241 | larnpammqe mmrnqdrals nlesipggyn alrrmytdiq epmlsaaqqeq fggnpfaslv |
| 301 | sntssgegsq psrtenrdpl pnpwapqtsq sssassgtas tvggttgsta sgtsgqstta |
| 361 | pnlvpgvgas mfntpgmqsl lqqitenpql mqnmlsapym rsmmqslsqn pdlaaqmmln |
| 421 | nplfagnpql qeqmrqqlpt flqqmqnpdt lsamsnpram gallqiqqgl qtlateapgl |
| 481 | ipgftpglga lgstggssgt ngsnatpsen tsptagttep ghqqfiqqm1 galagvnpql |
| 541 | qnpevrfqqq leqlsamgfl nreanlgali atggdinaai erllgsqps |

SEQ ID NO: 9 Human UBQLN1 Variant 2 cDNA Sequence (NM_053067.2,
CDS region from position 575-2260)

| | |
|---|---|
| 1 | aatttcgcta accgtgacgt catctcggtg cgccgttaca gcgcttaact ggaaactgcg |
| 61 | atttagtttt tggtgaaagg agtggaaaaa aaccatgaac tgacctaccc cgccccattt |
| 121 | tgcttttagt ccttcacact ggcttcgcct gcactttggg caatggagcg ggaaaggaaa |
| 181 | aggcacgcag gctgagctca aagtcgcgcc agccggggat gactcggcga cgccgccagg |
| 241 | cgcgttaccc ggcgtgctcc gcgcggcgcc ggcgaaggga cgtgggggaa ggggcaggga |
| 301 | ggaggaagcg gtggctgctg cggatgtcgg tgtgagcgag cggcgcctga acacacggcg |
| 361 | gctgccgagc gcctgacccg ggcctgcgcc agagcctgca ccgagctccg gggccccaca |
| 421 | cccgctacgg tggccctgcg cccgttgcta ctgaggcggc gtgctctgca ttcttcgctg |
| 481 | tccaggcctg ccggctctgg tgtctgctgg ctcctccttg ctcgcctgct ccctcctgct |
| 541 | tgcctgagtc accgccgccg ccgccgccac agccatggcc gagagtggtg aaagcggcgg |
| 601 | tcctccgggc tcccaggata gcgccgccgg agccgaaggt gctggcgccc ccgcggccgc |
| 661 | tgcctccgcg gagcccaaaa tcatgaaagt caccgtgaag accccgaagg aaaaggagga |
| 721 | attcgccgtg cccgagaata gctccgtcca gcagtttaag gaagaaatct ctaaacgttt |
| 781 | taaatcacat actgaccaac ttgtgttgat atttgctgga aaaattttga aagatcaaga |
| 841 | taccttgagt cagcatggaa ttcatgatgg acttactgtt caccttgtca ttaaaacaca |
| 901 | aaacaggcct caggatcatt cagctcagca aacaaataca gctggaagca atgttactac |
| 961 | atcatcaact cctaatagta actctacatc tggttctgct actagcaacc cttttggttt |
| 1021 | aggtggcctt gggggacttg caggtctgag tagcttgggt ttgaatacta ccaacttctc |
| 1081 | tgaactacag agtcagatgc agcgacaact tttgtctaac cctgaaatga tggtccagat |
| 1141 | catggaaaat ccctttgttc agagcatgct ctcaaatcct gacctgatga gacagttaat |
| 1201 | tatggccaat ccacaaatgc agcagttgat acagagaaat ccagaaatta gtcatatgtt |
| 1261 | gaataatcca gatataatga gacaaacgtt ggaacttgcc aggaatccag caatgatgca |
| 1321 | ggagatgatg aggaaccagg accgagcttt gagcaaccta gaaagcatcc cagggggata |
| 1381 | taatgcttta aggcgcatgt acacagatat tcaggaacca atgctgagtg ctgcacaaga |
| 1441 | gcagtttggt ggtaatccat ttgcttcctt ggtgagcaat acatcctctg gtgaaggtag |
| 1501 | tcaaccttcc cgtacagaaa atagagatcc actacccaat ccatgggctc cacagacttc |
| 1561 | ccagagttca tcagcttcca gcggcactgc cagcactgtg ggtggcacta ctggtagtac |
| 1621 | tgccagtggc acttctgggc agagtactac tgcgccaaat ttggtgcctg gagtaggagc |
| 1681 | tagtatgttc aacacaccag gaatgcagag cttgttgcaa caaataactg aaaacccaca |
| 1741 | actgatgcaa aacatgttgt ctgcccccta catgagaagc atgatgcagt cactaagcca |
| 1801 | gaatcctgac cttgctgcac agatgcagaa tcctgataca ctatcagcaa tgtcaaaccc |
| 1861 | tagagcaatg caggccttgt tacagattca gcagggttta cagacattag caacggaagc |
| 1921 | cccgggcctc atcccagggt ttactcctgg cttgggggca ttaggaagca ctggaggctc |
| 1981 | ttcgggaact aatggatcta acgccacacc tagtgaaaac acaagtccca cagcaggaac |
| 2041 | cactgaacct ggacatcagc agtttattca gcagatgctg caggctcttg ctggagtaaa |
| 2101 | tcctcagcta cagaatccag aagtcagatt tcagcaacaa ctggaacaac tcagtgcaat |
| 2161 | gggatttttg aaccgtgaag caaacttgca agctctaata gcaacaggag gtgatatcaa |
| 2221 | tgcagctatt gaaaggttac tgggctccca gccatcatag cagcatttct gtatcttgaa |
| 2281 | aaaatgtaat ttatttttga taacggctct taaactttaa aatacctgct ttatttcatt |
| 2341 | ttgactcttg gaattctgtg ctgttataaa caaacccaat atgatgcatt ttaaggtgga |
| 2401 | gtacagtaag atgtgtgggt ttttctgtat ttttcttttc tggaacagtg ggaattaagg |
| 2461 | ctactgcatg catcacttct gcatttattg taatttttta aaaacatcac cttttatagt |
| 2521 | tgggtgacca gattttgtcc tgcatctgtc cagtttattt gctttttaaa cattagccta |
| 2581 | tggtagtaat ttatgtagaa taaaagcatt aaaaagaagc aaatcatttg cactctataa |
| 2641 | tttgtggtac agtattgctt attgtgactt tggcatgcat ttttgcaaac aatgctgtaa |
| 2701 | gatttatact actgataatt ttgttttatt tgtatacaat atagagtatg cacatttggg |
| 2761 | actgcatttc tggaaacata ctgcaatagg ctctctgagc aaaacacctg taactaaaaa |
| 2821 | agtgaagata agaaaatact cttaaagctg agtatttcct aattgtatag aatcttacag |
| 2881 | catctttgac aaacatctcc cagcaaaagt gccggttagt caggtttgtt gaaaatacag |
| 2941 | tagaaaagct gattctggtt atctctttaa ggacaattaa ttgtacagac acataatgta |
| 3001 | acattgtctc aacattcatt cacagattga ctgtaaatta ccttaatctt tgtgcagact |
| 3061 | gaaggaacac tgtagtatac cccaaagtgc atttgcctag gacttctcag cttctcccat |
| 3121 | aggtagttta acaggcatta aaatttgtaa ttgaaatgtt gctttcactg aaaaagtgtc |
| 3181 | ttgatgtttc agttattttt aatcgccata aaaaaataga actatctttt gggtttatct |
| 3241 | gttttctcat gcacaggcaa tacacaaatt taaaatgagt tgtgagccaa ttgtttctga |
| 3301 | agtgttttgg tagttctatt aagaaatagt taaatattgt gcttttcaga gcctcagaga |
| 3361 | aagggggacg gggtgggggg gtggggcagc ggaatctgtc ctggatgggg ccagcttaaa |
| 3421 | taatactggc aaccaagatt ctgttaggat ttctgtgcat atagtgtagt aaagaagtat |
| 3481 | cattcagggg tgaaaaacaa agagccgttt taatgatgtt gagtacattt ggctgtttta |
| 3541 | tagccttttt cttccctccc ccaaagaatt ctgtttgcct aactcccaaa ctgttggggt |
| 3601 | ggtacattcc tttaggacca attaaaacat aattgagggt cagtgataca tttggctgac |
| 3661 | tctggttcag tattctctta ggtgattata ttctctcatg tacagttaca ggaaattaaa |
| 3721 | atgttaaagt aacctaaaat gaattcagac caataaaatc aagggaaata caagttgatt |
| 3781 | gcattacttc tgtatgttgc ttgctattaa aaaggttaag aggccaggtt acccaccagt |
| 3841 | ccttgcactg ttctgacact ttccccagga ggaaaacaag tacaaaggtt acggtggagg |
| 3901 | cataagtaga agagattgtt aagaagggta ttcatgtgtc tttgctcttt ctgctttatg |
| 3961 | cctcagtttg gtttaaaaac ttctgtactg gcaaatggtg gtattcagtg tgggatagtg |
| 4021 | tcataactaa tttgacaatt tattaatcat aaaataacaa taaatctcta gcttttacac |
| 4081 | ttgaaaaaaa aaa |

SEQ ID NO: 10 Human UBQLN1 Isoform 2 Amino Acid Sequence (NP_444295.1)

| | |
|---|---|
| 1 | maesgesggp pgsqdsaaga egagapaaaa saepkimkvt vktpkekeef avpenssvqq |
| 61 | fkeeiskrfk shtdqlvlif agkilkdqdt lsqhgihdgl tvhlviktqn rpqdhsaqqt |
| 121 | ntagsnvtts stpnsnstsg satsnpfglg glgglaglss lglnttnfse lqsqmqrqll |
| 181 | snpemmvqim enpfvqsmls npdlmrqlim anpqmqqliq rnpeishmln npdimrqtle |
| 241 | larnpammqe mmrnqdrals nlesipggyn alrrmytdiq epmlsaaqeq fggnpfaslv |
| 301 | sntssgegsq psrtenrdpl pnpwapqtsq sssassgtas tvggttgsta sgtsgqstta |
| 361 | pnlvpgvgas mfntpgmqsl lqqitenpql mqnmlsapym rsmmqslsqn pdlaaqmqnp |
| 421 | dtlsamsnpr amqallqiqq glqtlateap glipgftpgl galgstggss gtngsnatps |
| 481 | entsptagtt epghqqfiqq mlqalagvnp qlqnpevrfq qqleqlsamg flnreanlqa |
| 541 | liatggdina aierllgsqp s |

SEQ ID NO: 11 Mouse UBQLN1 Variant 1 cDNA Sequence (NM_026842.4,
CDS region from position 218-1966)

| | |
|---|---|
| 1 | agagtgccct gcgcggcgcc ggcgacgagc gtgggggagg tgcgggagga ggaagcggcg |
| 61 | gcgacgcgga gagtccccgc gtccacacgg cagccgatcg cccacccggg gcctgcaccc |
| 121 | gcagcccgcg tccacgcacc ctcggcggcc ccgcagcctg tcacgcctcg cggacccggc |
| 181 | cgcagcccgc agtgtcgcgc tcgcctgagt caccgccatg gccgagagcg cagagagcgg |
| 241 | cggcccaccg ggcgcgcagg acagtgcggc cgacggcggc cccgcagagc ccaagatcat |
| 301 | gaaggtcacg gtgaagacgc ctaaagagaa ggaggagttt gccgtgcccg agaacagctc |
| 361 | ggtccagcag ttcaaagagg aaatctctaa acgtttcaaa tctcatattg accaacttgt |
| 421 | gttgatattt gctggaaaaa ttttaaaaga tcaagatact ctgagtcagc atgggattca |
| 481 | tgatggactt acggttcacc ttgtcatcaa aacacagaac aggccgcaag ataattcagc |
| 541 | tcagcaaaca aatgcccctg gaagcactgt gaccagctca ccagctcccg atagcaaccc |
| 601 | cacgtctggt tctgctgcta acagctcctt tggcgtaggc ggacttggag gacttgcagg |
| 661 | tcttagcagc ttgggtttaa acaccaccaa cttctctgaa ctccagagcc aaatgcagcg |
| 721 | gcaacttttg tctaaccctg aaatgatggt ccagatcatg gaaaatccct ttgtccagag |
| 781 | catgctctca aatcctgacc tgatgcggca attgattatg gccaatccac agatgcaaca |
| 841 | gttgatacag agaaatccag agattagtca tatgcttaat aatccagata taatgagaca |
| 901 | aacactggaa cttgcgagaa atccagcgat gatgcaggaa atgatgagaa accaggaccg |
| 961 | agccttgagc aacctagaaa gtattcctgg gggctataat gccttacggc gcatgtacac |
| 1021 | cgatatccag gagcctatgc tgaatgctgc tcaagaacag tttggtggta atccatttgc |
| 1081 | gtccttagtg agcagttcgt cctcagcaga agggactcag ccttctcgaa cagaaaatag |
| 1141 | ggatccgctc cccaacccgt gggctccgca gacttcccag agctcaccag cctccggtac |
| 1201 | cactggcagc accactaaca ccatgagcac ctctggtggc actgccacca gtacccctgc |
| 1261 | tggacagagc acctcgggcc caagcttggt gcctggcgca ggagctagta tgttcaacac |
| 1321 | ccccggaatg cagagcctgc tgcagcagat aactgagaac ccacagctca tgcagaacat |
| 1381 | gctgtccgcc ccatacatga ggagcatgct gcagtccctg agccagaacc ccgaccttgc |
| 1441 | tgcgcagatg atgctgaata atcccctatt tgctggaaat cctcagcttc aagaacaaat |
| 1501 | gagacaacag ctcccaactt tcctccaaca aatgcagaac cccgacacgc tctcagcgat |
| 1561 | gtcgaatcct agagccatgc aggcgctgct gcagatccag cagggcttgc agacgctggc |
| 1621 | cacagaagct cctggcctca tcccagggtt tactcctggc ttggcagcag gcaattctgg |
| 1681 | aggctcttcc ggaaccaatg cacctagcac tgcacctagt gaagatacga acccccaagg |
| 1741 | gggcactgct gagccaggcc accagcagtt tatccagcag atgctgcagg cccttgcggg |
| 1801 | ggtgaatcct cagctgcaga gtccagaagt cagatttcag caacaactgg aacaactcag |
| 1861 | tgccatggga ttcttgaacc gtgaagcaaa cctgcaagct ctgatagcca cagggggcga |
| 1921 | catcaatgca gcaatcgaaa ggttgctggg ctcccagccg tcatagcagc atctctctat |
| 1981 | cctgagaaaa tgtaatttat ttttgataac ggctcttaaa tctttaaaat aacatgcttt |
| 2041 | atttcattgg gattctgtgc tgttataaag gaaaccaata ggaagtatct ggaggcagag |
| 2101 | tgcagtacga cgggcgggtt tctgtgtctt tttggaacag tgggaatcaa tgtttcttct |
| 2161 | ggttaaggct gctgcatgca tcaaacactt ggcatttatt gtaattttta aaaaaatatc |
| 2221 | accttttcta attgggtgaa caaatttttg tcctgcatct gtccaactaa cttgcttttt |
| 2281 | aaacataacc ctatggtagt aatttatgta gaataaaggc attaaaaagc agatcatttg |
| 2341 | cgcgccccat gatttctggt acaatagtgc ttgttgtgac tttggcatgc actttgcaaa |
| 2401 | tgatgctgta agatttatac tcctgacaat tttgtttatt tgtatacaat acaaattatt |
| 2461 | gcacatttct aggccatcta agcaaaattt ctgtaactaa aaaagtacaa taaggattcc |
| 2521 | taattgtgta gaatcttaca gcatctttga taaacatctc agcagaagtg ccatttagtc |
| 2581 | tgtgtggaaa ataaagaaaa actaactctg gttatctctt tagggacatt taattgtaca |
| 2641 | gacaacataa tgtaacaata tctcagcatt atataatggc tgtaaattac ctttccttgt |
| 2701 | gccgactgga ggagcactgt atcatgctct gatgtgtttg ctggggcgtc ctgctccccg |
| 2761 | taggtgtcgt ctacgcagca ttgagtttat tgaaatgtcg ctttcactgg aagtgcggat |
| 2821 | gtttcggttc taattgccat ataaaagtag agctgtctct tgggtttatc agctttctca |
| 2881 | ttcataggca atacctcgat tttaaatgat tagtgagcca cttgtttctc aagtgtttgg |
| 2941 | ctagttctat taagaaatag ttgaatgtcg tgctgttcgg agcctcagaa gggggtgggg |
| 3001 | gaggggtgaa acagaatcta ttctggattg ggccagcctg aactaggtaa tactggcaat |
| 3061 | taagattctg tgaagattct atgcatatag tagtgtcatt tgggtgaaaa agagccgttt |
| 3121 | taacaatttt gagtacgttt ggctattcta tagcctcttt cttccttccc caaagacgtt |
| 3181 | ctacttgcct aactctcaaa ctgttggggt gatacattcc tttgggacca gttaaaatac |
| 3241 | agcgttaggg tcggtgacat atgtgactgg ctctggttca gtattctctt atcttaggtc |
| 3301 | actgtagtct ctcttgtatg gttgcccaaa attataatgt taaataatct aaagtgagtt |
| 3361 | cagacaagta aaatcaaggg aaatagaagg tgaattctat aacttctgta agttgcttga |
| 3421 | tattaaaagg ttgagaggca ggttggcccc agactgtata cttttctgac actttacctg |
| 3481 | ggaagaaaac atgtacaaag gtcagggtgg aggcatgact gaggaggtcg gcacagcctg |
| 3541 | catgtgtgtg ctctctctgc cttacgcctc agtctgtggt tggaagactt tgtactggtg |
| 3601 | aatggcaata tgcaggtggg gcagtgtcaa aagcagtttg atttattaat ctccaaataa |
| 3661 | caataaatct ccagctttta cacttgaaaa aaa |

SEQ ID NO: 12 Mouse UBQLN1 Isoform 1 Amino Acid Sequence (NP_081118.4)

| | |
|---|---|
| 1 | maesaesggp pgaqdsaadg gpaepkimkv tvktpkekee favpenssvq qfkeeiskrf |
| 61 | kshidqlvli fagkilkdqd tlsqhgihdg ltvhlviktq nrpqdnsaqq tnapgstvts |
| 121 | spapdsnpts gsaanssfgv gglgglagls slglnttnfs elqsqmqrql lsnpemmvqi |
| 181 | menpfvqsml snpdlmrqli manpqmqqli qrnpeishml nnpdimrqtl elarnpammq |
| 241 | emmrnqdral snlesipggy nalrrmytdi qepmlnaaqe qfggnpfasl vsssssaegt |
| 301 | qpsrtenrdp lpnpwapqts qsspasgttg sttntmstsg gtatstpagq stsgpslvpg |
| 361 | agasmfntpg mqsllqqite npqlmqnmls apymrsmlqs lsqnpdlaaq mmlnnplfag |
| 421 | npqlqeqmrq qlptflqqmq npdtlsamsn pramgallqi qqglqtlate apglipgftp |
| 481 | glaagnsggs sgtnapstap sedtnpqggt aepghqqfiq gmlgalagvn pqlqspevrf |
| 541 | qqqleqlsam gflnreanlq aliatggdin aaierllgsq ps |

SEQ ID NO: 13 Mouse UBQLN1 Variant 2 cDNA Sequence (NM_152234.2,
CDS region from position 218-1882)

| | |
|---|---|
| 1 | agagtgccct gcgcggcgcc ggcgacgagc gtgggggagg tgcgggagga ggaagcggcg |
| 61 | gcgacgcgga gagtccccgc gtccacacgg cagccgatcg cccacccggg gcctgcaccc |
| 121 | gcagcccgcg tccacgcacc ctcggcggcc ccgcagcctg tcacgcctcg cggacccggc |
| 181 | cgcagcccgc agtgtcgcgc tcgcctgagt caccgccatg gccgagagcg cagagagcgg |
| 241 | cggcccaccg ggcgcgcagg acagtgcggc cgacggcggc cccgcagagc ccaagatcat |
| 301 | gaaggtcacg gtgaagacgc ctaaagagaa ggaggagttt gccgtgcccg agaacagctc |
| 361 | ggtccagcag ttcaaagagg aaatctctaa acgtttcaaa tctcatattg accaacttgt |
| 421 | gttgatattt gctggaaaaa ttttaaaaga tcaagatact ctgagtcagc atgggattca |
| 481 | tgatggactt acggttcacc ttgtcatcaa aacacagaac aggccgcaag ataattcagc |
| 541 | tcagcaaaca aatgcccctg gaagcactgt gaccagctca ccagctcccg atagcaaccc |
| 601 | cacgtctggt tctgctgcta acagctcctt tggcgtaggc ggacttggag gacttgcagg |
| 661 | tcttagcagc ttgggtttaa acaccaccaa cttctctgaa ctccagagcc aaatgcagcg |
| 721 | gcaacttttg tctaaccctg aaatgatggt ccagatcatg gaaaatccct ttgtccagag |
| 781 | catgctctca aatcctgacc tgatgcggca attgattatg gccaatccac agatgcaaca |
| 841 | gttgatacag agaaatccag agattagtca tatgcttaat aatccagata taatgagaca |
| 901 | aacactggaa cttgcgagaa atccagcgat gatgcaggaa atgatgagaa accaggaccg |
| 961 | agccttgagc aacctagaaa gtattcctgg gggctataat gccttacggc gcatgtacac |
| 1021 | cgatatccag gagcctatgc tgaatgctgc tcaagaacag tttggtggta atccatttgc |
| 1081 | gtccttagtg agcagttcgt cctcagcaga agggactcag ccttctcgaa cagaaaatag |
| 1141 | ggatccgctc cccaacccgt gggctccgca gacttcccag agctcaccag cctccggtac |
| 1201 | cactggcagc accactaaca ccatgagcac ctctggtggc actgccacca gtacccctgc |
| 1261 | tggacagagc acctcgggcc caagcttggt gcctggcgca ggagctagta tgttcaacac |
| 1321 | ccccggaatg cagagcctgc tgcagcagat aactgagaac ccacagctca tgcagaacat |
| 1381 | gctgtccgcc ccatacatga ggagcatgct gcagtccctg agccagaacc ccgaccttgc |
| 1441 | tgcgcagatg cagaaccccg acacgctctc agcgatgtcg aatcctagag ccatgcaggc |
| 1501 | gctgctgcag atccagcagg gcttgcagac gctggccaca gaagctcctg gcctcatccc |
| 1561 | agggtttact cctggcttgg cagcaggcaa ttctggaggc tcttccggaa ccaatgcacc |
| 1621 | tagcactgca cctagtgaag atacgaaccc ccaagggggc actgctgagc caggccacca |
| 1681 | gcagtttatc cagcagatgc tgcaggccct tgcgggggtg aatcctcagc tgcagagtcc |
| 1741 | agaagtcaga tttcagcaac aactggaaca actcagtgcc atgggattct tgaaccgtga |
| 1801 | agcaaacctg caagctctga tagccacagg gggcgacatc aatgcagcaa tcgaaaggtt |
| 1861 | gctgggctcc cagccgtcat agcagcatct ctctatcctg agaaaatgta atttattttt |
| 1921 | gataacggct cttaaatctt taaaataaca tgctttattt cattgggatt ctgtgctgtt |
| 1981 | ataaaggaaa ccaataggaa gtatctggag gcagagtgca gtacgacggg cgggtttctg |
| 2041 | tgtctttttg gaacagtggg aatcaatgtt tcttctggtt aaggctgctg catgcatcaa |
| 2101 | acacttggca tttattgtaa tttttaaaaa aatatcacct tttctaattg ggtgaacaaa |
| 2161 | tttttgtcct gcatctgtcc aactaacttg ctttttaaac ataaccctat ggtagtaatt |
| 2221 | tatgtagaat aaaggcatta aaaagcagat catttgcgcg ccccatgatt tctggtacaa |
| 2281 | tagtgcttgt tgtgactttg gcatgcactt tgcaaatgat gctgtaagat ttatactcct |
| 2341 | gacaattttg tttatttgta tacaatacaa attattgcac atttctaggc catctaagca |
| 2401 | aaatttctgt aactaaaaaa gtacaataag gattcctaat tgtgtagaat cttacagcat |
| 2461 | ctttgataaa catctcagca gaagtgccat ttagtctgtg tggaaaataa agaaaaacta |
| 2521 | actctggtta tctctttagg gacatttaat tgtacagaca acataatgta acaatatctc |
| 2581 | agcattatat aatggctgta aattaccttt ccttgtgccg actggaggag cactgtatca |
| 2641 | tgctctgatg tgtttgctgg ggcgtcctgc tccccgtagg tgtcgtctac gcagcattga |
| 2701 | gtttattgaa atgtcgcttt cactggaagt gcggatgttt cggttctaat tgccatataa |
| 2761 | aagtagagct gtctcttggg tttatcagct ttctcattca taggcaatac ctcgatttta |
| 2821 | aatgattagt gagccacttg tttctcaagt gtttggctag ttctattaag aaatagttga |
| 2881 | atgtcgtgct gttcggagcc tcagaagggg gtgggggagg ggtgaaacag aatctattct |
| 2941 | ggattgggcc agcctgaact aggtaatact ggcaattaag attctgtgaa gattctatgc |
| 3001 | atatagtagt gtcatttggg tgaaaaagag ccgttttaac aattttgagt acgtttggct |
| 3061 | attctatagc ctctttcttc cttccccaaa gacgttctac ttgcctaact ctcaaactgt |
| 3121 | tggggtgata cattcctttg ggaccagtta aaatacagcg ttagggtcgg tgacatatgt |
| 3181 | gactggctct ggttcagtat tctcttatct taggtcactg tagtctctct tgtatggttg |
| 3241 | cccaaaatta taatgttaaa taatctaaag tgagttcaga caagtaaaat caagggaaat |
| 3301 | agaaggtgaa ttctataact tctgtaagtt gcttgatatt aaaaggttga gaggcaggtt |
| 3361 | ggccccagac tgtatacttt tctgacactt tacctgggaa gaaaacatgt acaaaggtca |
| 3421 | gggtggaggc atgactgagg aggtcggcac agcctgcatg tgtgtgctct ctctgcctta |
| 3481 | cgcctcagtc tgtggttgga agactttgta ctggtgaatg gcaatatgca ggtggggcag |
| 3541 | tgtcaaaagc agtttgattt attaatctcc aaataacaat aaatctccag cttttacact |
| 3601 | tgaaaaaaa |

SEQ ID NO: 14 Mouse UBQLN1 Isoform 2 Amino Acid Sequence (NP_689420.1)

| | |
|---|---|
| 1 | maesaesggp pgaqdsaadg gpaepkimkv tvktpkekee favpenssvq qfkeeiskrf |
| 61 | kshidqlvli fagkilkdqd tlsqhgihdg ltvhlviktq nrpqdnsaqq tnapgstvts |
| 121 | spapdsnpts gsaanssfgv gglgglagls slglnttnfs elqsqmqrql lsnpemmvqi |
| 181 | menpfvqsml snpdlmrgli manpqmqqli qrnpeishml nnpdimrqtl elarnpammq |
| 241 | emmrnqdral snlesipggy nalrrmytdi qepmlnaage qfggnpfasl vsssssaegt |
| 301 | qpsrtenrdp lpnpwapqts qsspasgttg sttntmstsg gtatstpagq stsgpslvpg |
| 361 | agasmfntpg mqsllqqite npqlmqnmls apymrsmlqs lsqnpdlaaq mqnpdtlsam |
| 421 | snpramqall qiqqglqtla teapglipgf tpglaagnsg gssgtnapst apsedtnpqg |
| 481 | gtaepghqqf iqqmlqalag vnpqlqspev rfqqqleqls amgflnrean lqaliatggd |
| 541 | inaaierllg sqps |

SEQ ID NO: 15 Human HSP90B1 cDNA Sequence (NM 003299.2, CDS region
from position 183-2594)

| | |
|---|---|
| 1 | gattggtggg ttcatgtttc ccgtcccccg cccgcgggaa gtgggggtga aaagcggccc |
| 61 | gacctgcttg cggtgtagtg ggcggaccgc gcggctggag gtgtgaggat ccgaacccag |
| 121 | gggtgggggg tggaggcggc tcctgcgatc gaaggggact tgagactcac cggccgcacg |
| 181 | ccatgagggc cctgtgggtg ctgggcctct gctgcgtcct gctgaccttc gggtcggtca |
| 241 | gagctgacga tgaagttgat gtggatggta cagtagaaga ggatctgggt aaaagtagag |
| 301 | aaggatcaag gacggatgat gaagtagtac agagagagga agaagctatt cagttggatg |
| 361 | gattaaatgc atcacaaata agagaactta gagagaagtc ggaaaagttt gccttccaag |
| 421 | ccgaagttaa cagaatgatg aaacttatca tcaattcatt gtataaaaat aaagagattt |
| 481 | tcctgagaga actgatttca aatgcttctg atgctttaga taagataagg ctaatatcac |
| 541 | tgactgatga aaatgctctt tctggaaatg aggaactaac agtcaaaatt aagtgtgata |
| 601 | aggagaagaa cctgctgcat gtcacagaca ccggtgtagg aatgaccaga gaagagttgg |
| 661 | ttaaaaacct tggtaccata gccaaatctg ggacaagcga gtttttaaac aaaatgactg |
| 721 | aagcacagga agatggccag tcaacttctg aattgattgg ccagtttggt gtcggtttct |
| 781 | attccgcctt ccttgtagca gataaggtta ttgtcacttc aaaacacaac aacgataccc |
| 841 | agcacatctg ggagtctgac tccaatgaat tttctgtaat tgctgaccca agaggaaaca |
| 901 | ctctaggacg gggaacgaca attacccttg tcttaaaaga agaagcatct gattaccttg |
| 961 | aattggatac aattaaaaat ctcgtcaaaa aatattcaca gttcataaac tttcctattt |
| 1021 | atgtatggag cagcaagact gaaactgttg aggagcccat ggaggaagaa gaagcagcca |
| 1081 | aagaagagaa agaagaatct gatgatgaag ctgcagtaga ggaagaagaa gaagaaaaga |
| 1141 | aaccaaagac taaaaaagtt gaaaaaactg tctgggactg ggaacttatg aatgatatca |
| 1201 | aaccaatatg gcagagacca tcaaaagaag tagaagaaga tgaatacaaa gctttctaca |
| 1261 | aatcattttc aaaggaaagt gatgacccca tggcttatat tcactttact gctgaagggg |
| 1321 | aagttacctt caaatcaatt ttatttgtac ccacatctgc tccacgtggt ctgtttgacg |
| 1381 | aatatggatc taaaaagagc gattacatta agctctatgt gcgccgtgta ttcatcacag |
| 1441 | acgacttcca tgatatgatg cctaaatacc tcaattttgt caagggtgtg gtggactcag |
| 1501 | atgatctccc cttgaatgtt tcccgcgaga ctcttcagca acataaactg cttaaggtga |
| 1561 | ttaggaagaa gcttgttcgt aaaacgctgg acatgatcaa gaagattgct gatgataaat |
| 1621 | acaatgatac tttttggaaa gaatttggta ccaacatcaa gcttggtgtg attgaagacc |
| 1681 | actcgaatcg aacacgtctt gctaaacttc ttaggttcca gtcttctcat catccaactg |
| 1741 | acattactag cctagaccag tatgtggaaa gaatgaagga aaaacaagac aaaatctact |
| 1801 | tcatggctgg gtccagcaga aaagaggctg aatcttctcc atttgttgag cgacttctga |
| 1861 | aaaagggcta tgaagttatt tacctcacag aacctgtgga tgaatactgt attcaggccc |
| 1921 | ttcccgaatt tgatgggaag aggttccaga atgttgccaa ggaaggagtg aagttcgatg |
| 1981 | aaagtgagaa aactaaggag agtcgtgaag cagttgagaa agaatttgag cctctgctga |
| 2041 | attggatgaa agataaagcc cttaaggaca agattgaaaa ggctgtggtg tctcagcgcc |
| 2101 | tgacagaatc tccgtgtgct ttggtggcca gccagtacgg atggtctggc aacatggaga |
| 2161 | gaatcatgaa agcacaagcg taccaaacgg gcaaggacat ctctacaaat tactatgcga |
| 2221 | gtcagaagaa aacatttgaa attaatccca gacacccgct gatcagagac atgcttcgac |
| 2281 | gaattaagga agatgaagat gataaaacag ttttggatct tgctgtggtt ttgtttgaaa |
| 2341 | cagcaacgct tcggtcaggg tatcttttac cagacactaa agcatatgga gatagaatag |
| 2401 | aaagaatgct tcgcctcagt ttgaacattg accctgatgc aaaggtggaa gaagagcccg |
| 2461 | aagaagaacc tgaagagaca gcagaagaca caacagaaga cacagagcaa gacgaagatg |
| 2521 | aagaaatgga tgtgggaaca gatgaagaag aagaaacagc aaaggaatct acagctgaaa |
| 2581 | aagatgaatt gtaaattata ctctcaccat ttggatcctg tgtggagagg gaatgtgaaa |
| 2641 | tttacatcat ttctttttgg gagagacttg ttttggatgc cccctaatcc ccttctcccc |
| 2701 | tgcactgtaa aatgtgggat tatgggtcac aggaaaaagt gggtttttta gttgaatttt |
| 2761 | ttttaacatt cctcatgaat gtaaatttgt actatttaac tgactattct tgatgtaaaa |
| 2821 | tcttgtcatg tgtataaaaa taaaaaagat cccaaatact caaaaaaaaa aaaaaaaaa |

SEO ID NO: 16 Human HSP90B1 Amino Acid Sequence (NP_003290.1)

| | |
|---|---|
| 1 | mralwvlglc cvlltfgsvr addevdvdgt veedlgksre gsrtddevvq reeealqldg |
| 61 | lnasqlrelr eksekfafqa evnrmmklii nslyknkeif lrelisnasd aldkirlisl |
| 121 | tdenalsgne eltvkikcdk eknllhvtdt gvgmtreelv knlgtiaksg tseflnkmte |
| 181 | aqedgqstse ligqfgvgfy saflvadkvi vtskhnndtq hiwesdsnef sviadprgnt |
| 241 | lgrgttitlv lkeeasdyle ldtiknlvkk ysqfinfpiy vwssktetve epmeeeeaak |
| 301 | eekeesddea aveeeeeekk pktkkvektv wdwelmndik piwqrpskev eedeykafyk |
| 361 | sfskesddpm ayihftaege vtfksilfvp tsaprglfde ygskksdyik lyvrrvfitd |
| 421 | dfhdmmpkyl nfvkgvvdsd dlplnvsret lqqhkllkvi rkklvrktld mikkiaddky |
| 481 | ndtfwkefgt niklgviedh snrtrlakll rfqsshhptd itsldqyver mkekqdkiyf |
| 541 | magssrkeae sspfverllk kgyeviylte pvdeyciqal pefdgkrfqn vakegvkfde |
| 601 | sektkesrea vekefeplln wmkdkalkdk iekavvsqrl tespcalvas qyqwsgnmer |
| 661 | imkaqayqtg kdistnyyas qkktfeinpr hplirdmlrr ikededdktv ldlavvlfet |
| 721 | atlrsgyllp dtkaygdrie rmlrlslnid pdakveeepe eepeetaedt tedtegdede |
| 781 | emdvgtdeee etakestaek del |

SEQ ID NO: 17 Mouse HSP90B1 cDNA Sequence (NM_011631.1, CDS region
from position 90-2498)

| | |
|---|---|
| 1 | tggaggtgtg aggagcttag actcgggatt gggggggtgg aggcggctcc tgagaccgaa |
| 61 | aaggacttgc gactcgccgg ccacgcacca tgagggtcct gtgggtgttg ggcctctgct |
| 121 | gtgtcctgct gaccttcggg ttcgtcagag ctgatgatga agtcgacgtg gatggcacag |
| 181 | tggaagagga cctgggtaaa agccgagaag gctcaaggac agatgatgaa gttgtgcaga |
| 241 | gagaggaaga agctattcag ttggatgggt taaacgcatc acagataaga gaacttagag |
| 301 | aaaaatctga aaagttcgcc ttccaagctg aagtgaacag gatgatgaaa cttatcatca |
| 361 | attctttgta taaaaataaa gagattttcc tgagagaact gatttcaaat gcttctgatg |
| 421 | ctttagacaa gataaggctc atctccctaa ctgatgaaaa tgcactcgct ggaaatgagg |
| 481 | agttaacggt caagattaag tgtgacaaag agaaaaacct gctgcatgtc acagacacgg |
| 541 | gtgtaggaat gactagagag gagttggtta aaaatctcgg caccatagcc aaatctggaa |
| 601 | caagcgagtt tttaaacaaa atgacagaag ctcaagaaga tggtcagtca acctctgaac |
| 661 | tgattggcca gtttggtgtc ggtttttatt ctgccttcct tgtagcagat aaggtcattg |
| 721 | tcacatcgaa acacaacaat gatacccagc acatctggga atcagactcc aatgaattct |
| 781 | ctgtaattgc tgacccaaga ggaaacacac taggtcgtgg aacaacaatt actcttgtct |
| 841 | taaaagaaga agcatctgat taccttgaat tggacacaat taaaaatctt gtcaggaagt |
| 901 | actctcagtt catcaacttt cccatctacg tgtggagtag caagacagag actgttgagg |
| 961 | agcccttgga agaagatgaa gcagcaaaag aagagaaaga agaatctgat gatgaagctg |
| 1021 | cagtagagga ggaagaagaa gaaaagaaac caaaaactaa gaaagttgaa aaaactgtgt |
| 1081 | gggattggga acttatgaat gatatcaaac caatatggca gagaccatcc aaagaagtag |
| 1141 | aagaagacga atacaaagct ttctacaaat cattttcaaa ggaaagtgat gaccccatgg |
| 1201 | cttatatcca cttcactgca gaaggggagg tcaccttcaa gtcgattttg tttgtaccca |
| 1261 | catctgcacc tcgaggtctg tttgatgaat atggatctaa gaagagtgat tatattaagc |
| 1321 | tgtatgtacg ccgcgtattc atcacagatg acttccatga tatgatgccc aaatacctta |
| 1381 | attttgtcaa aggtgttgtg gattccgatg atctccccct caatgtttcc cgtgagactc |
| 1441 | ttcagcaaca taaattgctc aaggtgatta ggaagaagct tgtccgaaaa actctggaca |
| 1501 | tgatcaagaa gattgctgat gagaagtata acgacacttt ctggaaggag ttcggcacga |
| 1561 | atatcaagct tggtgtgatt gaagaccact caaatcgaac acggcttgct aaacttctta |
| 1621 | ggttccagtc ttctcaccat tcaactgaca ttactagttt agaccagtat gtggaaagaa |
| 1681 | tgaaggaaaa acaggacaaa atctacttca tggctgggtc aagcagaaag gaggcggaat |
| 1741 | cttctccatt tgttgagaga cttctgaaga agggctatga agtcatttat ctcacagagc |
| 1801 | ctgtggatga atactgcatt caggctcttc ccgagtttga tgggaagagg tttcagaatg |
| 1861 | ttgccaaaga aggggtgaag tttgatgaga gtgagaaaac taaagaaagt cgggaagcaa |
| 1921 | cagagaagga gtttgaacct ctgctcaact ggatgaaaga taaggccctc aaggacaaga |
| 1981 | tagaaaaggc tgtggtgtcg cagcgcctca cagagtctcc ctgtgctctt gtggccagtc |
| 2041 | agtatggatg gtctggcaac atggagagga tcatgaaggc acaagcatac cagacgggca |
| 2101 | aggacatctc tacaaattac tatgccagtc aaaagaaaac gttcgaaatc aatcctagac |
| 2161 | acccactgat cagagacatg ttgcggcgga ttaaggaaga tgaagatgac aagacagtca |
| 2221 | tggatcttgc tgtagttttg tttgaaacgg caacacttcg gtcaggatat cttctaccag |
| 2281 | acaccaaggc gtatggagat agaatagaaa gaatgcttcg cctcagttta aacattgacc |
| 2341 | ctgaagcaca ggtggaggaa gaaccagaag aagagcctga agacacctca gaagacgcag |
| 2401 | aagactcaga gcaggatgag ggagaagaga tggatgcagg gacagaagaa gaagaggagg |
| 2461 | aaacagaaaa ggaatctaca gagaaggatg aattgtaaat tatactctcg ctatgaatcc |
| 2521 | cgtgtggaga gggaatgtga agttttgaag tcatttcttt tgagagactt gttttggatg |
| 2581 | cttccccaag cctccttctc ccctgcactg taaaatgttg ggattatggg tcacaggaag |
| 2641 | aagtggtttt tttagttgaa ttttttttaa cattcctcct gaatgtaaat ttgtactatt |
| 2701 | taactgacta ttggtgtaaa atcttgtcat gtgtataaaa ataaaaaaag atcccaaat |

SEQ ID NO: 18 Mouse HSP90B1 Amino Acid Sequence (NP_035761.1)

| | |
|---|---|
| 1 | mrvlwvlglc cvlltfgfvr addevdvdgt veedlgksre gsrtddevvq reeeaiqldg |
| 61 | lnasqirelr eksekfafqa evnrmmklii nslyknkeif lrelisnasd aldkirlisl |
| 121 | tdenalagne eltvkikcdk eknllhvtdt gvgmtreelv knlgtiaksg tseflnkmte |
| 181 | aqedgqstse ligqfgvgfy saflvadkvi vtskhnndtq hiwesdsnef sviadprgnt |
| 241 | lgrgttitiv lkeeasdyle ldtiknlvrk ysqfinfpiy vwssktetve epleedeaak |
| 301 | eekeesddea aveeeeeekk pktkkvektv wdwelmndik piwqrpskev eedeykafyk |
| 361 | sfskesddpm ayihftaege vtfksilfvp tsaprglfde ygskksdyik lyvrrvfitd |
| 421 | dfhdmmpkyl nfvkgvvdsd dlplnvsret lqqhkllkvi rkklvrktld mikkiadeky |
| 481 | ndtfwkefgt niklgviedh snrtrlakll rfqsshhstd itsldqyver mkekqdkiyf |
| 541 | magssrkeae sspfverllk kgyeviylte pvdeyciqal pefdgkrfqn vakegvkfde |
| 601 | sektkesrea tekefeplln wmkdkalkdk iekavvsqrl tespcalvas qygwsgnmer |
| 661 | imkaqayqtg kdistnyyas qkktfeinpr hplirdmlrr ikededdktv mdlavvlfet |
| 721 | atlrsgyllp dtkaygdrie rmlrlslnid peaqveeepe eepedtseda edseqdegee |
| 781 | mdagteeeee etekestekd el |

Included in Table 1 are RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 1, or a portion thereof. Such nucleic acid molecules can have a function of the full-length nucleic acid as described further herein.
Included in Table 1 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1, or a portion thereof. Such polypeptides can have a function of the full-length polypeptide as described further herein.

### II. Subjects

In one embodiment, the subject for whom predicted likelihood of efficacy of an inhibitor of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and an immunotherapy combination treatment is determined, is a mammal (e.g., mouse, rat, primate, non-human mammal, domestic animal, such as a dog, cat, cow, horse, and the like), and is preferably a human. In another embodiment, the subject is an animal model of cancer. For example, the animal model can be an orthotopic xenograft animal model of a human-derived cancer.

In another embodiment of the methods of the present invention, the subject has not undergone treatment, such as chemotherapy, radiation therapy, targeted therapy, and/or immunotherapies. In still another embodiment, the subject has undergone treatment, such as chemotherapy, radiation therapy, targeted therapy, and/or immunotherapies.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

The methods of the present invention can be used to determine the responsiveness to biomarmer inhibitors and immunotherapy combination treatment of many different cancers in subjects such as those described herein.

### III. Sample Collection, Preparation and Separation

In some embodiments, biomarker amount and/or activity measurement(s) in a sample from a subject is compared to a predetermined control (standard) sample. The sample from the subject is typically from a diseased tissue, such as cancer cells or tissues. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects. In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount and/or activity measurement(s) used to, by way of example only, evaluate a subject that may be selected for treatment (e.g., based on the number of genomic mutations and/or the number of genomic mutations causing non-functional proteins for DNA repair genes), evaluate a response to an inhibitor of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and an immunotherapy combination treatment, and/or evaluate a response to an inhibitor of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and an immunotherapy combination treatment with one or more additional anti-cancer therapies. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements.

In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of anti-cancer therapy. Post-treatment biomarker measurement can be made at any time after initiation of anti-cancer therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of anti-cancer therapy, and even longer toward indefinitely for continued monitoring. Treatment can comprise anti-cancer therapy, such as a therapeutic regimen comprising an inhibitor of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and immunotherapy combination treatment alone or in combination with other anti-cancer agents, such as with immune checkpoint inhibitors.

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of biomarker amount and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### IV. Biomarker Nucleic Acids and Polypeptides

One aspect of the present invention pertains to the use of isolated nucleic acid molecules that correspond to biomarker nucleic acids that encode a biomarker polypeptide or a portion of such a polypeptide. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A biomarker nucleic acid molecule of the present invention can be isolated using standard molecular biology techniques and the sequence information in the database records described herein. Using all or a portion of such nucleic acid sequences, nucleic acid molecules of the present invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the present invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis.

Furthermore, oligonucleotides corresponding to all or a portion of a nucleic acid molecule of the present invention can be prepared by standard synthetic techniques, e.g., using an automated DNA synthesizer.

Moreover, a nucleic acid molecule of the present invention can comprise only a portion of a nucleic acid sequence, wherein the full length nucleic acid sequence comprises a marker of the present invention or which encodes a polypeptide corresponding to a marker of the present invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a biomarker nucleic acid sequence. Probes based on the sequence of a biomarker nucleic acid molecule can be used to detect transcripts or genomic sequences corresponding to one or more markers of the present invention. The probe comprises a label group attached thereto, e.g., a radioisotope, a fluorescent compound, an enzyme, or an enzyme co-factor.

A biomarker nucleic acid molecules that differ, due to degeneracy of the genetic code, from the nucleotide sequence of nucleic acid molecules encoding a protein which corresponds to the biomarker, and thus encode the same protein, are also contemplated.

In addition, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. For example, biomarker alleles can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

As used herein, the terms “gene” and “recombinant gene” refer to nucleic acid molecules comprising an open reading frame encoding a polypeptide corresponding to a marker of the present invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the present invention.

In another embodiment, a biomarker nucleic acid molecule is at least 7, 15, 20, 25, 30, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2200, 2400, 2600, 2800, 3000, 3500, 4000, 4500, or more nucleotides in length and hybridizes under stringent conditions to a nucleic acid molecule corresponding to a marker of the present invention or to a nucleic acid molecule encoding a protein corresponding to a marker of the present invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule of the present invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the present invention pertains to nucleic acid molecules encoding a polypeptide of the present invention that contain changes in amino acid residues that are not essential for activity. Such polypeptides differ in amino acid sequence from the naturally-occurring proteins which correspond to the markers of the present invention, yet retain biological activity. In one embodiment, a biomarker protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of a biomarker protein described herein.

An isolated nucleic acid molecule encoding a variant protein can be created by introducing one or more nucleotide substitutions, additions or deletions into the nucleotide sequence of nucleic acids of the present invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In some embodiments, the present invention further contemplates the use of anti-biomarker antisense nucleic acid molecules, i.e., molecules which are complementary to a sense nucleic acid of the present invention, e.g., complementary to the coding strand of a double-stranded cDNA molecule corresponding to a marker of the present invention or complementary to an mRNA sequence corresponding to a marker of the present invention. Accordingly, an antisense nucleic acid molecule of the present invention can hydrogen bond to (i.e. anneal with) a sense nucleic acid of the present invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the present invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the present invention are typically administered to a subject or generated in situ such that they hybridize with or bind to cellular mRNA and/or genomic DNA encoding a polypeptide corresponding to a selected marker of the present invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the present invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the present invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The present invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the present invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the present invention can be used to select a catalytic RNA having a specific ribonuclease activity from a pool of RNA molecules (see, e.g., Bartel and Szostak, 1993, Science 261:1411-1418).

The present invention also encompasses nucleic acid molecules which form triple helical structures. For example, expression of a biomarker protein can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

Another aspect of the present invention pertains to the use of biomarker proteins and biologically active portions thereof. In one embodiment, the native polypeptide corresponding to a marker can be isolated from cells or tissue sources by an appropriate purification scheme using standard protein purification techniques. In another embodiment, polypeptides corresponding to a marker of the present invention are produced by recombinant DNA techniques. Alternative to recombinant expression, a polypeptide corresponding to a marker of the present invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a biomarker polypeptide include polypeptides comprising amino acid sequences sufficiently identical to or derived from a biomarker protein amino acid sequence described herein, but which includes fewer amino acids than the full length protein, and exhibit at least one activity of the corresponding full-length protein. Typically, biologically active portions comprise a domain or motif with at least one activity of the corresponding protein. A biologically active portion of a protein of the present invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the present invention.

Preferred polypeptides have an amino acid sequence of a biomarker protein encoded by a nucleic acid molecule described herein. Other useful proteins are substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the present invention. BLAST protein searches can be performed with the XBLAST program, score=50, wordlength=3 to obtain amino acid sequences homologous to a protein molecules of the present invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The present invention also provides chimeric or fusion proteins corresponding to a biomarker protein. As used herein, a “chimeric protein” or “fusion protein” comprises all or part (preferably a biologically active part) of a polypeptide corresponding to a marker of the present invention operably linked to a heterologous polypeptide (i.e., a polypeptide other than the polypeptide corresponding to the marker). Within the fusion protein, the term “operably linked” is intended to indicate that the polypeptide of the present invention and the heterologous polypeptide are fused in-frame to each other. The heterologous polypeptide can be fused to the amino-terminus or the carboxyl-terminus of the polypeptide of the present invention.

One useful fusion protein is a GST fusion protein in which a polypeptide corresponding to a marker of the present invention is fused to the carboxyl terminus of GST sequences. Such fusion proteins can facilitate the purification of a recombinant polypeptide of the present invention.

In another embodiment, the fusion protein contains a heterologous signal sequence, immunoglobulin fusion protein, toxin, or other useful protein sequence. Chimeric and fusion proteins of the present invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the present invention can be cloned into such an expression vector such that the fusion moiety is linked in-frame to the polypeptide of the present invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the present invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the biomarker polypeptides described herein. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a biomarker protein which function as either agonists (mimetics) or as antagonists can be identified by screening combinatorial libraries of mutants, e.g., truncation mutants, of the protein of the present invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the present invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the present invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the present invention (Arkin and Yourvan, 1992, Proc. Natl. Acad. Sci. USA 89:7811-7815; Delgrave et al., 1993, Protein Engineering 6(3):327-331).

An isolated polypeptide or a fragment thereof (or a nucleic acid encoding such a polypeptide) corresponding to one or more biomarkers of the invention, including the biomarkers listed in Table 1 or fragments thereof, can be used as an immunogen to generate antibodies that bind to said immunogen, using standard techniques for polyclonal and monoclonal antibody preparation according to well-known methods in the art. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

In some embodiments, the immunotherapy utilizes an inhibitor of at least one immune checkpoint, such as an antibody binds substantially specifically to an immune checkpoint, such as PD-1, and inhibits or blocks its immunoinhibitory function, such as by interrupting its interaction with a binding partner of the immune checkpoint, such as PD-L1 and/or PD-L2 binding partners of PD-1. In one embodiment, an antibody, especially an intrbody, binds substantially specifically to one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and inhibits or blocks its biological function, such as by interrupting its interaction with a substrate. In another embodiment, an antibody, especially an intrbody, binds substantially specifically to a binding partner of one or more biomarkers listed in Table 1, such as substrates of one or more biomarkers described herein, and inhibits or blocks its biological function, such as by interrupting its interaction to the one or more biomarkers.

For example, a polypeptide immunogen typically is used to prepare antibodies by immunizing a suitable subject (e.g., rabbit, goat, mouse or other mammal) with the immunogen. A preferred animal is a mouse deficient in the desired target antigen. For example, a PD-1 knockout mouse if the desired antibody is an anti-PD-1 antibody, may be used. This results in a wider spectrum of antibody recognition possibilities as antibodies reactive to common mouse and human epitopes are not removed by tolerance mechanisms. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497) (see also Brown et al. (1981) J. Immunol. 127:539-46; Brown et al. (1980)J. Biol. Chem. 255:4980-83; Yeh et al. (1976) Proc. Natl. Acad. Sci. 76:2927-31; Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well-known (see generally Kenneth, R. H. in Monoclonal Antibodies: A New Dimension In Biological Analyses, Plenum Publishing Corp., New York, N.Y. (1980); Lerner, E. A. (1981) Yale J Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically. In some embodiments, the immunization is performed in a cell or animal host that has a knockout of a target antigen of interest (e.g., does not produce the antigen prior to immunization).

Any of the many well-known protocols used for fusing lymphocytes and immortalized cell lines can be applied for the purpose of generating a monoclonal antibody against one or more biomarkers of the invention, including the biomarkers listed in Table 1, or a fragment thereof (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

As an alternative to preparing monoclonal antibody-secreting hybridomas, a monoclonal specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurJZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Since it is well-known in the art that antibody heavy and light chain CDR3 domains play a particularly important role in the binding specificity/affinity of an antibody for an antigen, the recombinant monoclonal antibodies of the present invention prepared as set forth above preferably comprise the heavy and light chain CDR3s of variable regions of the antibodies described herein and well-known in the art. Similarly, the antibodies can further comprise the CDR2s of variable regions of said antibodies. The antibodies can further comprise the CDR1s of variable regions of said antibodies. In other embodiments, the antibodies can comprise any combinations of the CDRs.

The CDR1, 2, and/or 3 regions of the engineered antibodies described above can comprise the exact amino acid sequence(s) as those of variable regions of the present invention described herein. However, the ordinarily skilled artisan will appreciate that some deviation from the exact CDR sequences may be possible while still retaining the ability of the antibody, especially an introbody, to bind a desired target, such as one or more biomarkers listed in Table 1, and/or a binding partner thereof, either alone or in combination with an immunotherapy, such as one or more biomarkers listed in Table 1, biomarker binding partners/substrates, or an immunotherapy effectively (e.g., conservative sequence modifications). Accordingly, in another embodiment, the engineered antibody may be composed of one or more CDRs that are, for example, 50%, 60%, 70%, 80%, 85%, 90%, 91%, 92%, 93%, 94%, 95%, 96%, 97%, 98%, 99%, or 99.5% identical to one or more CDRs of the present invention described herein or otherwise publicly available.

For example, the structural features of non-human or human antibodies (e.g., a rat anti-mouse/anti-human antibody) can be used to create structurally related human antibodies, especially introbodies, that retain at least one functional property of the antibodies of the present invention, such as binding to one or more biomarkers listed in Table 1, biomarker binding partners/substrates, and/or an immune checkpoint. Another functional property includes inhibiting binding of the original known, non-human or human antibodies in a competition ELISA assay.

Antibodies, immunoglobulins, and polypeptides of the invention can be used in an isolated (e.g., purified) form or contained in a vector, such as a membrane or lipid vesicle (e.g. a liposome). Moreover, amino acid sequence modification(s) of the antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Similarly, modifications and changes may be made in the structure of the antibodies described herein, and in the DNA sequences encoding them, and still obtain a functional molecule that encodes an antibody and polypeptide with desirable characteristics. For example, antibody glycosylation patterns can be modulated to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr et al. (1987) and by Edge et al. (1981). Enzymatic cleavage of carbohydrate moieties on antibodies can be achieved by the use of a variety of endo- and exo-glycosidases as described by Thotakura et al. (1987).

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins of the present invention with heterologous agents can be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present invention features antibodies conjugated to a therapeutic moiety, such as a cytotoxin, a drug, and/or a radioisotope. When conjugated to a cytotoxin, these antibody conjugates are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). An antibody of the present invention can be conjugated to a radioisotope, e.g., radioactive iodine, to generate cytotoxic radiopharmaceuticals for treating a related disorder, such as a cancer.

Conjugated antibodies, in addition to therapeutic utility, can be useful for diagnostically or prognostically to monitor polypeptide levels in tissue as part of a clinical testing procedure, e.g., to determine the efficacy of a given treatment regimen. Detection can be facilitated by coupling (i e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include ¹²⁵I, ¹³¹I, ³⁵S, or ³H. [0134] As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody, as well as indirect labeling of the antibody by reactivity with a detectable substance.

The antibody conjugates of the present invention can be used to modify a given biological response. The therapeutic moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, Pseudomonas exotoxin, or diphtheria toxin; a protein such as tumor necrosis factor or interferon-.gamma.; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other cytokines or growth factors.

In one embodiment, an antibody for use in the instant invention is a bispecific or multispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a polypeptide or a fragment thereof of one or more biomarkers of the invention, including one or more biomarkers listed in Table 1, or a fragment thereof. In one embodiment, the bispecific antibody could specifically bind to both a polypeptide or a fragment thereof and its natural binding partner(s) or a fragment(s) thereof.

Techniques for modulating antibodies, such as humanization, conjugation, recombinant techniques, and the like are well-known in the art.

In another aspect of this invention, peptides or peptide mimetics can be used to antagonize the activity of one or more biomarkers of the invention, including one or more biomarkers listed in Table 1, or a fragment(s) thereof. In one embodiment, variants of one or more biomarkers listed in Table 1 which function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. USA 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes one or more biomarkers of the invention, including one or more biomarkers listed in Table 1, or a fragment thereof. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences described herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well-known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides described herein can be used therapeutically to treat disease, e.g., by altering costimulation in a patient.

Peptidomimetics (Fauchere (1986) Adv. Drug Res. 15:29; Veber and Freidinger (1985) TINS p. 392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH₂S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190)J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivatization (e.g., labeling) of peptidomimetics should not substantially interfere with the desired biological or pharmacological activity of the peptidomimetic.

Also encompassed by the present invention are small molecules which can modulate (either enhance or inhibit) interactions, e.g., between biomarkers described herein or listed in Table 1 and their natural binding partners. The small molecules of the present invention can be obtained using any of the numerous approaches in combinatorial library methods known in the art, including: spatially addressable parallel solid phase or solution phase libraries; synthetic library methods requiring deconvolution; the ‘one-bead one-compound’library method; and synthetic library methods using affinity chromatography selection. (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.). Compounds can be screened in cell based or non-cell based assays. Compounds can be screened in pools (e.g. multiple compounds in each testing sample) or as individual compounds.

Chimeric or fusion proteins can be prepared for the inhibitors of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and/or agents for the immunotherapies described herein, such as inhibitors to the biomarkers of the invention, including the biomarkers listed in Table 1, or fragments thereof. As used herein, a “chimeric protein” or “fusion protein” comprises one or more biomarkers of the invention, including one or more biomarkers listed in Table 1, or a fragment thereof, operatively linked to another polypeptide having an amino acid sequence corresponding to a protein which is not substantially homologous to the respective biomarker. In a preferred embodiment, the fusion protein comprises at least one biologically active portion of one or more biomarkers of the invention, including one or more biomarkers listed in Table 1, or fragments thereof. Within the fusion protein, the term “operatively linked” is intended to indicate that the biomarker sequences and the non-biomarker sequences are fused in-frame to each other in such a way as to preserve functions exhibited when expressed independently of the fusion. The “another” sequences can be fused to the N-terminus or C-terminus of the biomarker sequences, respectively.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. In another preferred embodiment, the first peptide consists of a portion of a biologically active molecule (e.g. the extracellular portion of the polypeptide or the ligand binding portion). The second peptide can include an immunoglobulin constant region, for example, a human Cγ1 domain or Cy4 domain (e.g., the hinge, CH2 and CH3 regions of human IgCγ 1, or human IgCγ4, see e.g., Capon et al. U.S. Pat. Nos. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well-known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992).

The fusion proteins of the invention can be used as immunogens to produce antibodies in a subject. Such antibodies may be used to purify the respective natural polypeptides from which the fusion proteins were generated, or in screening assays to identify polypeptides which inhibit the interactions between one or more biomarkers polypeptide or a fragment thereof and its natural binding partner(s) or a fragment(s) thereof.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g., bind) under cellular conditions, with cellular nucleic acids (e.g., small non-coding RNAS such as miRNAs, pre-miRNAs, pri-miRNAs, miRNA*, anti-miRNA, a miRNA binding site, a variant and/or functional variant thereof, cellular mRNAs or a fragments thereof). In one embodiment, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, one or more biomarkers of the invention, including one or more biomarkers listed in Table 1, or fragment(s) thereof. In one embodiment, the small nucleic acids or antisense oligonucleotides or derivatives thereof are small RNAs (e.g., microRNAs) or complements of small RNAs. In another embodiment, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21, 20, 19, 18, 17, 16, 15, or 10 nucleotides in length. In another embodiment, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 2-5, 5-10, 10-20, 10-30 or more nucleic acids comprising or capable of expressing small nucleic acids or antisense oligonucleotides or derivatives thereof.

In one embodiment, binding may be by conventional base pair complementarity, or, for example, in the case of binding to DNA duplexes, through specific interactions in the major groove of the double helix. In general, “antisense” refers to the range of techniques generally employed in the art, and includes any process that relies on specific binding to oligonucleotide sequences.

It is well-known in the art that modifications can be made to the sequence of a miRNA or a pre-miRNA without disrupting miRNA activity. As used herein, the term “functional variant” of a miRNA sequence refers to an oligonucleotide sequence that varies from the natural miRNA sequence, but retains one or more functional characteristics of the miRNA (e.g. cancer cell proliferation inhibition, induction of cancer cell apoptosis, enhancement of cancer cell susceptibility to chemotherapeutic agents, specific miRNA target inhibition). In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant is capable of hybridizing to one or more target sequences of the miRNA.

miRNAs and their corresponding stem-loop sequences described herein may be found in miRBase, an online searchable database of miRNA sequences and annotation, found on the world wide web at microrna.sanger.ac.uk. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

In some embodiments, miRNA sequences of the invention may be associated with a second RNA sequence that may be located on the same RNA molecule or on a separate RNA molecule as the miRNA sequence. In such cases, the miRNA sequence may be referred to as the active strand, while the second RNA sequence, which is at least partially complementary to the miRNA sequence, may be referred to as the complementary strand. The active and complementary strands are hybridized to create a double-stranded RNA that is similar to a naturally occurring miRNA precursor. The activity of a miRNA may be optimized by maximizing uptake of the active strand and minimizing uptake of the complementary strand by the miRNA protein complex that regulates gene translation. This can be done through modification and/or design of the complementary strand.

In some embodiments, the complementary strand is modified so that a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules known in the art, including NH2, NHCOCH3, and biotin.

In another embodiment, the uptake of the complementary strand by the miRNA pathway is reduced by incorporating nucleotides with sugar modifications in the first 2-6 nucleotides of the complementary strand. It should be noted that such sugar modifications can be combined with the 5′ terminal modifications described above to further enhance miRNA activities.

In some embodiments, the complementary strand is designed so that nucleotides in the 3′ end of the complementary strand are not complementary to the active strand. This results in double-strand hybrid RNAs that are stable at the 3′ end of the active strand but relatively unstable at the 5′ end of the active strand. This difference in stability enhances the uptake of the active strand by the miRNA pathway, while reducing uptake of the complementary strand, thereby enhancing miRNA activity.

Small nucleic acid and/or antisense constructs of the methods and compositions presented herein can be delivered, for example, as an expression plasmid which, when transcribed in the cell, produces RNA which is complementary to at least a unique portion of cellular nucleic acids (e.g., small RNAs, mRNA, and/or genomic DNA). Alternatively, the small nucleic acid molecules can produce RNA which encodes mRNA, miRNA, pre-miRNA, pri-miRNA, miRNA*, anti-miRNA, or a miRNA binding site, or a variant thereof. For example, selection of plasmids suitable for expressing the miRNAs, methods for inserting nucleic acid sequences into the plasmid, and methods of delivering the recombinant plasmid to the cells of interest are within the skill in the art. See, for example, Zeng et al. (2002) Mol. Cell 9:1327-1333; Tuschl (2002), Nat. Biotechnol. 20:446-448; Brummelkamp et al. (2002) Science 296:550-553; Miyagishi et al. (2002) Nat. Biotechnol. 20:497-500; Paddison et al. (2002) Genes Dev. 16:948-958; Lee et al. (2002) Nat. Biotechnol. 20:500-505; and Paul et al. (2002) Nat. Biotechnol. 20:505-508, the entire disclosures of which are herein incorporated by reference.

Alternatively, small nucleic acids and/or antisense constructs are oligonucleotide probes that are generated ex vivo and which, when introduced into the cell, results in hybridization with cellular nucleic acids. Such oligonucleotide probes are preferably modified oligonucleotides that are resistant to endogenous nucleases, e.g., exonucleases and/or endonucleases, and are therefore stable in vivo. Exemplary nucleic acid molecules for use as small nucleic acids and/or antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches may involve the design of oligonucleotides (either DNA or RNA) that are complementary to cellular nucleic acids (e.g., complementary to biomarkers listed in Table 1). Absolute complementarity is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a nucleic acid (e.g., RNA) it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner (1994) Nature 372:333). Therefore, oligonucleotides complementary to either the 5′ or 3′ untranslated, non-coding regions of genes could be used in an antisense approach to inhibit translation of endogenous mRNAs. Oligonucleotides complementary to the 5′ untranslated region of the mRNA may include the complement of the AUG start codon. Antisense oligonucleotides complementary to mRNA coding regions are less efficient inhibitors of translation but could also be used in accordance with the methods and compositions presented herein. Whether designed to hybridize to the 5′, 3′ or coding region of cellular mRNAs, small nucleic acids and/or antisense nucleic acids should be at least six nucleotides in length, and can be less than about 1000, 900, 800, 700, 600, 500, 400, 300, 200, 100, 50, 40, 30, 25, 24, 23, 22, 21, 20, 19, 18, 17, 16, 15, or 10 nucleotides in length.

Regardless of the choice of target sequence, it is preferred that in vitro studies are first performed to quantitate the ability of the antisense oligonucleotide to inhibit gene expression. In one embodiment these studies utilize controls that distinguish between antisense gene inhibition and nonspecific biological effects of oligonucleotides. In another embodiment these studies compare levels of the target nucleic acid or protein with that of an internal control nucleic acid or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Small nucleic acids and/or antisense oligonucleotides can be DNA or RNA or chimeric mixtures or derivatives or modified versions thereof, single-stranded or double-stranded. Small nucleic acids and/or antisense oligonucleotides can be modified at the base moiety, sugar moiety, or phosphate backbone, for example, to improve stability of the molecule, hybridization, etc., and may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. U.S.A. 84:648-652; PCT Publication No. WO88/09810) or the blood-brain barrier (see, e.g., PCT Publication No. WO89/10134), hybridization-triggered cleavage agents. (See, e.g., Krol et al. (1988) BioTech. 6:958-976) or intercalating agents. (See, e.g., Zon (1988) Pharm. Res. 5:539-549). To this end, small nucleic acids and/or antisense oligonucleotides may be conjugated to another molecule, e.g., a peptide, hybridization triggered cross-linking agent, transport agent, hybridization-triggered cleavage agent, etc.

Small nucleic acids and/or antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Small nucleic acids and/or antisense oligonucleotides may also comprise at least one modified sugar moiety selected from the group including but not limited to arabinose, 2-fluoroarabinose, xylulose, and hexose.

In certain embodiments, a compound comprises an oligonucleotide (e.g., a miRNA or miRNA encoding oligonucleotide) conjugated to one or more moieties which enhance the activity, cellular distribution or cellular uptake of the resulting oligonucleotide. In certain such embodiments, the moiety is a cholesterol moiety (e.g., antagomirs) or a lipid moiety or liposome conjugate. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to the oligonucleotide. In certain embodiments, a conjugate group is attached to the oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain such embodiments, the compound comprises the oligonucleotide having one or more stabilizing groups that are attached to one or both termini of the oligonucleotide to enhance properties such as, for example, nuclease stability. Included in stabilizing groups are cap structures. These terminal modifications protect the oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

Small nucleic acids and/or antisense oligonucleotides can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, small nucleic acids and/or antisense oligonucleotides comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In a further embodiment, small nucleic acids and/or antisense oligonucleotides are α-anomeric oligonucleotides. An a-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-O-methylribonucleotide (Inoue et al. (1987) Nucl. Acids Res. 15:6131-6148), or a chimeric RNA-DNA analogue (Inoue et al. (1987) FEBS Lett. 215:327-330).

Small nucleic acids and/or antisense oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451), etc. For example, an isolated miRNA can be chemically synthesized or recombinantly produced using methods known in the art. In some instances, miRNA are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), Cruachem (Glasgow, UK), and Exiqon (Vedbaek, Denmark).

Small nucleic acids and/or antisense oligonucleotides can be delivered to cells in vivo. A number of methods have been developed for delivering small nucleic acids and/or antisense oligonucleotides DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

In one embodiment, small nucleic acids and/or antisense oligonucleotides may comprise or be generated from double stranded small interfering RNAs (siRNAs), in which sequences fully complementary to cellular nucleic acids (e.g. mRNAs) sequences mediate degradation or in which sequences incompletely complementary to cellular nucleic acids (e.g., mRNAs) mediate translational repression when expressed within cells, or piwiRNAs. In another embodiment, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene. in vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nat. Biotechnol. 20:1006; and Brummelkamp et al. (2002) Science 296:550). Vectors that can be used are commercially available, e.g., from OligoEngine under the name pSuper RNAi System™ Ribozyme molecules designed to catalytically cleave cellular mRNA transcripts can also be used to prevent translation of cellular mRNAs and expression of cellular polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site specific recognition sequences can be used to destroy cellular mRNAs, the use of hammerhead ribozymes is preferred.

Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well-known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al. (1984) Science 224:574-578; Zaug et al. (1986) Science 231:470-475; Zaug et al. (1986) Nature 324:429-433; WO 88/04300; and Been et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Small nucleic acids (e.g., miRNAs, pre-miRNAs, pri-miRNAs, miRNA*, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well-known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp, 2nd edition (July 1999).

The modulatory agents described herein (e.g., antibodies, small molecules, peptides, fusion proteins, or small nucleic acids) can be incorporated into pharmaceutical compositions and administered to a subject in vivo. The compositions may contain a single such molecule or agent or any combination of agents described herein. “Single active agents” described herein can be combined with other pharmacologically active compounds (“second active agents”) known in the art according to the methods and compositions provided herein.

The production and use of biomarker nucleic acid and/or biomarker polypeptide molecules described herein can be facilitated by using standard recombinant techniques. In some embodiments, such techniques use vectors, preferably expression vectors, containing a nucleic acid encoding a biomarker polypeptide or a portion of such a polypeptide. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present invention is intended to include such other forms of expression vectors, such as viral vectors (e.g., replication defective retroviruses, adenoviruses and adeno-associated viruses), which serve equivalent functions.

The recombinant expression vectors of the present invention comprise a nucleic acid of the present invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the present invention can be introduced into host cells to thereby produce proteins or peptides, including fusion proteins or peptides, encoded by nucleic acids as described herein.

The recombinant expression vectors for use in the present invention can be designed for expression of a polypeptide corresponding to a marker of the present invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol. 185, Academic Press, San Diego, Calif., 1991). Target biomarker nucleic acid expression from the pTrc vector relies on host RNA polymerase transcription from a hybrid trp-lac fusion promoter. Target biomarker nucleic acid expression from the pET 11d vector relies on transcription from a T7 gn10-lac fusion promoter mediated by a co-expressed viral RNA polymerase (T7 gn1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the present invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the present invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The present invention further provides a recombinant expression vector comprising a DNA molecule cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the present invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes (see Weintraub et al., 1986, Trends in Genetics, Vol. 1(1)).

Another aspect of the present invention pertains to host cells into which a recombinant expression vector of the present invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

### V. Analyzing Biomarker Nucleic Acids and Polypeptides

Biomarker nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

a. Methods for Detection of Copy Number

Methods of evaluating the copy number of a biomarker nucleic acid are well-known to those of skill in the art. The presence or absence of chromosomal gain or loss can be evaluated simply by a determination of copy number of the regions or markers identified herein.

In one embodiment, a biological sample is tested for the presence of copy number changes in genomic loci containing the genomic marker. A copy number of at least 3, 4, 5, 6, 7, 8, 9, or 10 is predictive of poorer outcome of inhibitors of one or more biomarkers listed in Table 1 and immunotherapy combination treatments.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984) EMBO J. 3: 1227-1234; Pinkel (1988) Proc. Natl. Acad. Sci. USA 85: 9138-9142; EPO Pub. No. 430, 402; Methods in Molecular Biology, Vol. 33: In situ Hybridization Protocols, Choo, ed., Humana Press, Totowa, N.J. (1994), etc.) In another embodiment, the hybridization protocol of Pinkel, et al. (1998) Nature Genetics 20: 207-211, or of Kallioniemi (1992) Proc. Natl Acad Sci USA 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the present invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) and major copy proportion (MCP) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93; Li et al., (2008) MBC Bioinform. 9, 204-219) may also be used to identify regions of amplification or deletion.

b. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc. Natl. Acad. Sci. U.S.A. 86: 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Other known amplification methods which can be utilized herein include but are not limited to the so-called “NASBA” or “3 SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos. 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

c. Methods for Detection of Biomarker Protein Expression

The activity or level of a biomarker protein can be detected and/or quantified by detecting or quantifying the expressed polypeptide. The polypeptide can be detected and quantified by any of a number of means well-known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to inhibitors of one or more biomarkers listed in Table 1 and immunotherapy combination treatments. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a Kd of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0, 120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

d. Methods for Detection of Biomarker Structural Alterations

The following illustrative methods can be used to identify the presence of a structural alteration in a biomarker nucleic acid and/or biomarker polypeptide molecule in order to, for example, identify STUB1, UBQLN1, HSP90B1, or other biomarkers used in the immunotherapies described herein that are overexpressed, overfunctional, and the like.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

### 3. Anti-Cancer Therapies

The efficacy of inhibitors of one or more biomarkers listed in Table 1 and immunotherapy combination treatment is predicted according to biomarker amount and/or activity associated with a cancer in a subject according to the methods described herein. In one embodiment, such inhibitors and immunotherapy combination treatments (e.g., one or more inhibitors of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and immunotherapy combination treatment in combination with one or more additional anti-cancer therapies, such as another immune checkpoint inhibitor) can be administered, particularly if a subject has first been indicated as being a likely responder to the inhibitor of one or more biomarkers and immunotherapy combination treatment. In another embodiment, such biomarker inhibitor and immunotherapy combination treatment can be avoided once a subject is indicated as not being a likely responder to biomarker inhibitor(s) and immunotherapy combination treatment and an alternative treatment regimen, such as targeted and/or untargeted anti-cancer therapies can be administered. Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with anti-immune checkpoint therapy. In addition, any representative embodiment of an agent to modulate a particular target can be adapted to any other target described herein by the ordinarily skilled artisn (e.g., direct and indirect PD-1 inhibitors described herein can be applied to other immune checkpoint inhibitors and/or the one or more biomarkers listed in Table 1, such as monospecific antibodies, bispecific antibodies, non-activiting forms, small molecules, peptides, interfering nucleic acids, and the like).

The term “targeted therapy” refers to administration of agents that selectively interact with a chosen biomolecule to thereby treat cancer. One example includes immunotherapies such as immune checkpoint inhibitors, which are well-known in the art. For example, anti-PD-1 pathway agents, such as therapeutic monoclonal blocking antibodies, which are well-known in the art and described above, can be used to target tumor microenvironments and cells expressing unwanted components of the PD-1 pathway, such as PD-1, PD-L1, and/or PD-L2.

For example, the term “PD-1 pathway” refers to the PD-1 receptor and its ligands, PD-L1 and PD-L2. “PD-1 pathway inhibitors” block or otherwise reduce the interaction between PD-1 and one or both of its ligands such that the immunoinhibitory signaling otherwise generated by the interaction is blocked or otherwise reduced. Anti-immune checkpoint inhibitors can be direct or indirect. Direct anti-immune checkpoint inhibitors block or otherwise reduce the interaction between an immune checkpoint and at least one of its ligands. For example, PD-1 inhibitors can block PD-1 binding with one or both of its ligands. Direct PD-1 combination inhibitors are well-known in the art, especially since the natural binding partners of PD-1 (e.g., PD-L1 and PD-L2), PD-L1 (e.g., PD-1 and B7-1), and PD-L2 (e.g., PD-1 and RGMb) are known.

For example, agents which directly block the interaction between PD-1 and PD-L1, PD-1 and PD-L2, PD-1 and both PD-L1 and PD-L2, such as a bispecific antibody, can prevent inhibitory signaling and upregulate an immune response (i.e., as a PD-1 pathway inhibitor). Alternatively, agents that indirectly block the interaction between PD-1 and one or both of its ligands can prevent inhibitory signaling and upregulate an immune response. For example, B7-1 or a soluble form thereof, by binding to a PD-L1 polypeptide indirectly reduces the effective concentration of PD-L1 polypeptide available to bind to PD-1. Exemplary agents include monospecific or bispecific blocking antibodies against PD-1, PD-L1, and/or PD-L2 that block the interaction between the receptor and ligand(s); a non-activating form of PD-1, PD-L1, and/or PD-L2 (e.g., a dominant negative or soluble polypeptide), small molecules or peptides that block the interaction between PD-1, PD-L1, and/or PD-L2; fusion proteins (e.g. the extracellular portion of PD-1, PD-L1, and/or PD-L2, fused to the Fc portion of an antibody or immunoglobulin) that bind to PD-1, PD-L1, and/or PD-L2 and inhibit the interaction between the receptor and ligand(s); a non-activating form of a natural PD-1, PD-L2, and/or PD-L2 ligand, and a soluble form of a natural PD-1, PD-L2, and/or PD-L2 ligand.

Indirect anti-immune checkpoint inhibitors block or otherwise reduce the immunoinhibitory signaling generated by the interaction between the immune checkpoint and at least one of its ligands. For example, an inhibitor can block the interaction between PD-1 and one or both of its ligands without necessarily directly blocking the interaction between PD-1 and one or both of its ligands. For example, indirect inhibitors include intrabodies that bind the intracellular portion of PD-1 and/or PD-L1 required to signal to block or otherwise reduce the immunoinhibitory signaling. Similarly, nucleic acids that reduce the expression of PD-1, PD-L1, and/or PD-L2 can indirectly inhibit the interaction between PD-1 and one or both of its ligands by removing the availability of components for interaction. Such nucleic acid molecules can block PD-L1, PD-L2, and/or PD-L2 transcription or translation.

Similarly, agents which directly block the interaction between one or more biomarkers listed in Table 1 and their binding partners/substrates, and the like, can remove the inhibition to biomarker-regulated signaling and its downstream immune responses, such as increasing sensitivity to interferon signaling. Alternatively, agents that indirectly block the interaction between the one or more biomarkers and their binding partners/substrates can remove the inhibition to biomarker-regulated signaling and its downstream immune responses. For example, a truncated or dominant negative form of one or more biomarkers listed in Table 1, such as biomarker fragments without ubiquitin-proteasome pathway regulating activity, by binding to a biomarker substrate indirectly reduces the effective concentration of such substrate available to bind to the biomarker in cell. Exemplary agents include monospecific or bispecific blocking antibodies, especially intrbodies, against one or more biomarkers listed in Table 1 and/or biomarker substrate(s) that block the interaction between the biomarker and its substrate(s); a non-active form of one or more biomarkers listed in Table 1 and/or biomarker substrate(s) (e.g., a dominant negative polypeptide), small molecules or peptides that block the interaction between one or more biomarkers listed in Table 1 and its substrate(s) or the catalytic activity of one or more biomarkers listed in Table 1; and a non-activating form of a natural biomarker and/or its substrate(s).

Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In one embodiment, immunotherapy comprises adoptive cell-based immunotherapies. Well-known adoptive cell-based immunotherapeutic modalities, including, without limitation, Irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, patient-specific neoantigen vaccines, and the like.

In another embodiment, immunotherapy comprises non-cell-based immunotherapies. In one embodiment, compositions comprising antigens with or without vaccine-enhancing adjuvants are used. Such compositions exist in many well-known forms, such as peptide compositions, oncolytic viruses, recombinant antigen comprising fusion proteins, and the like. In still another embodiment, immunomodulatory interleukins, such as IL-2, IL-6, IL-7, IL-12, IL-17, IL-23, and the like, as well as modulators thereof (e.g., blocking antibodies or more potent or longer lasting forms) are used. In yet another embodiment, immunomodulatory cytokines, such as interferons, G-CSF, imiquimod, TNFalpha, and the like, as well as modulators thereof (e.g., blocking antibodies or more potent or longer lasting forms) are used. In another embodiment, immunomodulatory chemokines, such as CCL3, CCL26, and CXCL7, and the like, as well as modulators thereof (e.g., blocking antibodies or more potent or longer lasting forms) are used. In another embodiment, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

In still another embodiment, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C(indole-3-carbinol)/DIM(di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet another embodiment, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, agents and therapies other than immunotherapy or in combination thereof can be used with in combination with biomarker inhibitor/immunotherapies to stimulate an immune response to thereby treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. No. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et.al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, surgical intervention can occur to physically remove cancerous cells and/or tissues.

In still another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In yet another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early non-small cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with therapies may vary according to the particular therapeutic agent or combination thereof. An appropriate treatment time for a particular cancer therapeutic agent will be appreciated by the skilled artisan. The present invention contemplates the continued assessment of optimal treatment schedules for each cancer therapeutic agent, where the phenotype of the cancer of the subject as determined by the methods of the present invention is a factor in determining optimal treatment doses and schedules.

Any means for the introduction of a polynucleotide into mammals, human or non-human, or cells thereof may be adapted to the practice of this invention for the delivery of the various constructs of the present invention into the intended recipient. In one embodiment of the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well-known and any can be selected for a particular application. In one embodiment of the present invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide of the present invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

### 4. Clincal Efficacy

Clinical efficacy can be measured by any method known in the art. For example, the response to a therapy, such as inhibitors of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and immunotherapy combination treatment, relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to immunotherapies, such as anti-immune checkpoint therapies, are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any immunotherapy, such as anti-immune checkpoint therapy. The outcome measurement may be pathologic response to therapy given in the neoadjuvant setting. Alternatively, outcome measures, such as overall survival and disease-free survival can be monitored over a period of time for subjects following immunotherapies for whom biomarker measurement values are known. In certain embodiments, the same doses of immunotherapy agents, if any, are administered to each subject. In related embodiments, the doses administered are standard doses known in the art for those agents used in immunotherapies. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of an immunotherapy can be determined using methods such as those described in the Examples section.

### 5. Further Uses and Methods of the Present Invention

The compositions described herein can be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Screening Methods

One aspect of the present invention relates to screening assays, including non-cell based assays and xenograft animal model assays. In one embodiment, the assays provide a method for identifying whether a cancer is likely to respond to inhibitors of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and immunotherapy combination treatments, such as in a human by using a xenograft animal model assay, and/or whether an agent can inhibit the growth of or kill a cancer cell that is unlikely to respond to biomarker inhibitor and immunotherapy combination treatments.

In one embodiment, the present invention relates to assays for screening test agents which bind to, or modulate the biological activity of, at least one biomarker described herein (e.g., in the tables, figures, examples, or otherwise in the specification). In one embodiment, a method for identifying such an agent entails determining the ability of the agent to modulate, e.g. inhibit, the at least one biomarker described herein.

In one embodiment, an assay is a cell-free or cell-based assay, comprising contacting at least one biomarker described herein, with a test agent, and determining the ability of the test agent to modulate (e.g., inhibit) the enzymatic activity of the biomarker, such as by measuring direct binding of substrates or by measuring indirect parameters as described below.

For example, in a direct binding assay, biomarker protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies described herein can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to modulate the interaction between the biomarker and a substrate or a biomarker and its natural binding partner can be accomplished by determining the ability of the test agent to modulate the activity of a polypeptide or other product that functions downstream or upstream of its position within the signaling pathway (e.g., feedback loops). Such feedback loops are well-known in the art (see, for example, Chen and Guillemin (2009) Int. J. Tryptophan Res. 2:1-19).

The present invention further pertains to novel agents identified by the above-described screening assays. Accordingly, it is within the scope of this invention to further use an agent identified as described herein, such as in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

b. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the amount and/or activity level of a biomarker described herein in the context of a biological sample (e.g., blood, serum, cells, or tissue) to thereby determine whether an individual afflicted with a cancer is likely to respond to biomarker inhibitor and immunotherapy combination treatments, such as in a cancer. Such assays can be used for prognostic or predictive purpose alone, or can be coupled with a therapeutic intervention to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those in the tables, figures, examples, and otherwise described in the specification.

Another aspect of the present invention pertains to monitoring the influence of agents (e.g., drugs, compounds, and small nucleic acid-based molecules) on the expression or activity of a biomarker described herein. These and other agents are described in further detail in the following sections.

The skilled artisan will also appreciated that, in certain embodiments, the methods of the present invention implement a computer program and computer system. For example, a computer program can be used to perform the algorithms described herein. A computer system can also store and manipulate data generated by the methods of the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the non-cancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

c. Diagnostic Assays

The present invention provides, in part, methods, systems, and code for accurately classifying whether a biological sample is associated with a cancer that is likely to respond to biomarker inhibitor and immunotherapy combination treatments. In some embodiments, the present invention is useful for classifying a sample (e.g., from a subject) as associated with or at risk for responding to or not responding to biomarker inhibitor and immunotherapy combination treatments using a statistical algorithm and/or empirical data (e.g., the amount or activity of a biomarker described herein, such as in the tables, figures, examples, and otherwise described in the specification).

An exemplary method for detecting the amount or activity of a biomarker described herein, and thus useful for classifying whether a sample is likely or unlikely to respond to biomarker inhibitor and immunotherapy combination treatments involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely immunotherapy responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In another embodiment, the diagnosis of a subject is followed by administering to the individual a therapeutically effective amount of a defined treatment based upon the diagnosis.

In one embodiment, the methods further involve obtaining a control biological sample (e.g., biological sample from a subject who does not have a cancer or whose cancer is susceptible to biomarker inhibitor and immunotherapy combination treatments), a biological sample from the subject during remission, or a biological sample from the subject during treatment for developing a cancer progressing despite biomarker inhibitor and immunotherapy combination treatments.

d. Prognostic Assays

The diagnostic methods described herein can furthermore be utilized to identify subjects having or at risk of developing a cancer that is likely or unlikely to be responsive to biomarker inhibitor and immunotherapy combination treatments. The assays described herein, such as the preceding diagnostic assays or the following assays, can be utilized to identify a subject having or at risk of developing a disorder associated with a misregulation of the amount or activity of at least one biomarker described herein, such as in cancer. Alternatively, the prognostic assays can be utilized to identify a subject having or at risk for developing a disorder associated with a misregulation of the at least one biomarker described herein, such as in cancer. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with the aberrant biomarker expression or activity.

e. Treatment Methods

The therapeutic compositions described herein, such as the combination of biomarker inhibitor and immunotherapy, can be used in a variety of in vitro and in vivo therapeutic applications using the formulations and/or combinations described herein. In one embodiment, the therapeutic agents can be used to treat cancers determined to be responsive thereto. For example, single or multiple agents that inhibit or block both a biomarker inhibitor and a immunotherapy can be used to treat cancers in subjects identified as likely responders thereto.

Modulatory methods of the present invention involve contacting a cell, such as an immune cell with an agent that inhibits or blocks the expression and/or activity of one or more biomarkers listed in Table 1, such as one or more regulators of ubiquitin proteasome pathway (e.g., STIP1 homology and U-Box containing protein 1 (STUB1), ubiquilin-1 (UBQLN1), and heat shock protein 90 kDa beta member 1 (HSP90B1)), and an immunotherapy, such as an immune checkpoint inhibitor (e.g., PD-1). Exemplary agents useful in such methods are described above. Such agents can be administered in vitro or ex vivo (e.g., by contacting the cell with the agent) or, alternatively, in vivo (e.g., by administering the agent to a subject). As such, the present invention provides methods useful for treating an individual afflicted with a condition that would benefit from an increased immune response, such as an infection or a cancer like colorectal cancer.

Agents that upregulate immune responses can be in the form of enhancing an existing immune response or eliciting an initial immune response. Thus, enhancing an immune response using the subject compositions and methods is useful for treating cancer, but can also be useful for treating an infectious disease (e.g., bacteria, viruses, or parasites), a parasitic infection, and an immunosuppressive disease.

Exemplary infectious disorders include viral skin diseases, such as Herpes or shingles, in which case such an agent can be delivered topically to the skin. In addition, systemic viral diseases, such as influenza, the common cold, and encephalitis might be alleviated by systemic administration of such agents. In one preferred embodiment, agents that upregulate the immune response described herein are useful for modulating the arginase/iNOS balance during Trypanosoma cruzi infection in order to facilitate a protective immune response against the parasite.

Immune responses can also be enhanced in an infected patient through an ex vivo approach, for instance, by removing immune cells from the patient, contacting immune cells in vitro with an agent described herein and reintroducing the in vitro stimulated immune cells into the patient.

In certain instances, it may be desirable to further administer other agents that upregulate immune responses, for example, forms of other B7 family members that transduce signals via costimulatory receptors, in order to further augment the immune response. Such additional agents and therapies are described further below.

Agents that upregulate an immune response can be used prophylactically in vaccines against various polypeptides (e.g., polypeptides derived from pathogens). Immunity against a pathogen (e.g., a virus) can be induced by vaccinating with a viral protein along with an agent that upregulates an immune response, in an appropriate adjuvant.

In another embodiment, upregulation or enhancement of an immune response function, as described herein, is useful in the induction of tumor immunity.

In another embodiment, the immune response can be stimulated by the methods described herein, such that preexisting tolerance, clonal deletion, and/or exhaustion (e.g., T cell exhaustion) is overcome. For example, immune responses against antigens to which a subject cannot mount a significant immune response, e.g., to an autologous antigen, such as a tumor specific antigens can be induced by administering appropriate agents described herein that upregulate the immune response. In one embodiment, an autologous antigen, such as a tumor-specific antigen, can be coadministered. In another embodiment, the subject agents can be used as adjuvants to boost responses to foreign antigens in the process of active immunization.

In one embodiment, immune cells are obtained from a subject and cultured ex vivo in the presence of an agent as described herein, to expand the population of immune cells and/or to enhance immune cell activation. In a further embodiment the immune cells are then administered to a subject. Immune cells can be stimulated in vitro by, for example, providing to the immune cells a primary activation signal and a costimulatory signal, as is known in the art. Various agents can also be used to costimulate proliferation of immune cells. In one embodiment immune cells are cultured ex vivo according to the method described in PCT Application No. WO 94/29436. The costimulatory polypeptide can be soluble, attached to a cell membrane, or attached to a solid surface, such as a bead.

### 6. Administration of Agents

The immune modulating agents of the invention are administered to subjects in a biologically compatible form suitable for pharmaceutical administration in vivo, to enhance immune cell mediated immune responses. By “biologically compatible form suitable for administration in vivo” is meant a form to be administered in which any toxic effects are outweighed by the therapeutic effects. The term “subject” is intended to include living organisms in which an immune response can be elicited, e.g., mammals. Examples of subjects include humans, dogs, cats, mice, rats, and transgenic species thereof. Administration of an agent as described herein can be in any pharmacological form including a therapeutically active amount of an agent alone or in combination with a pharmaceutically acceptable carrier.

Administration of a therapeutically active amount of the therapeutic composition of the present invention is defined as an amount effective, at dosages and for periods of time necessary, to achieve the desired result. For example, a therapeutically active amount of an agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

Inhibiting or blocking expression and/or activity of one or more biomarkers listed in Table 1, alone or in combination with an immunotherapy, can be accomplished by combination therapy with the modulatory agents described herein. Combination therapy describes a therapy in which one or more biomarkers listed in Table 1 is inhibited or blocked with an immunotherapy simultaneously. This may be achieved by administration of the modulatory agent described herein with the immunotherapy simultaneously (e.g., in a combination dosage form or by simultaneous administration of single agents) or by administration of single inhibitory agent for one or more biomarkers listed in Table 1 and the immunotherapy, according to a schedule that results in effective amounts of each modulatory agent present in the patient at the same time.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) biomarker expression and/or activity, or expression and/or activity of the complex, or composition comprising an agent that modulates (e.g., inhibits) biomarker expression and/or activity, or expression and/or activity of the complex, which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity, or expression and/or activity of the complex encompassed by the present invention. These salts can be prepared in situ during the final isolation and purification of the therapeutic agents, or by separately reacting a purified therapeutic agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the therapeutic agents, or by separately reacting the purified therapeutic agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a therapeutic agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a therapeutic agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well-known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the therapeutic agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules of the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In one embodiment, an agent of the invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays.

### 7. Kits

The present invention also encompasses kits for detecting and/or modulating biomarkers described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### EXAMPLES

### Example 1: Materials and Methods for Examples 2-4

a. In Vivo CRISPR Screening in B16 Tumor Cells

A Cas9-expressing version of the B16 melanoma cell line was created and confirmed that it could edit DNA efficiently with CRISPR using sgRNAs targeting the PD-L1 gene. For screening the B16-Cas9 cell line, a library of 9,992 optimized sgRNAs was created to target 2,398 genes, selected from the GO term categories: kinase, phosphatase, cell surface, plasma membrane, antigen processing and presentation, immune system process, and chromatin remodeling. The transcript abundance of the genes in these categories were then filtered to include only those that were expressed >RPKM (log₂)=0.9. These genes were then ranked for expression in the B16 cell line using RNAseq to select for the top 2,398 expressed genes. The library was divided into 4 sub-pools, each containing one sgRNA per gene and 100 non-targeting control sgRNAs. The 4 sub-pools were screened individually and sgRNAs were delivered to B16-Cas9 cells via lentiviral infection at an infection rate of 30%. Transduced B16 cells were purified using a hCD19 reporter by positive magnetic selection (Miltenyi Biotech, Cambridge, Mass.) and then expanded in vitro before being implanted into animals. For each sub-pool, B16 cells were implanted into 10 TCRα^{-/-} mice, 10 WT mice treated with GVAX, and 10 WT mice treated with GVAX and PD-1 blockade (see below for treatment protocols). B16 cells transduced with libraries were also grown in vitro at approximately 2000× library coverage for the same time period as the animal experiment. Mice were sacrificed 12-14 days after tumor implantation tumor genomic DNA was prepared from whole tumor tissue using the Qiagen DNA Blood Midi kit (Qiagen, Hilden, Germany). PCR was used to amplify the sgRNA region and sequencing to determine sgRNA abundance was performed on an Illumina HiSeq system (Illumina, San Diego, Calif.). Significantly enriched or depleted sgRNAs from any comparison of conditions were identified using the STARS algorithm (Doench et al. (2014) Nat. Biotechnol. 32:1262-1267).

b. Animal Treatment and Tumor Challenges

The designs of these animal studies and procedures were approved by the Dana Farber Cancer Institute IACUC committee. Dana Farber's specific-pathogen free facility was used for the storage and care of all mice. Seven-week old wild-type female C57BL/6J mice were obtained from Jackson laboratories (Bar Harbor, Me.). A colony of B6.129S2-Tcra^{tm1Mom}/J (Tcra) T cell-deficient mice were bred on site. Mice were aged matched to be 7-12 weeks old at the time of tumor inoculation. For screening, 2.0×10⁶ library-transduced B16-Cas9 cells resuspended in Hanks Balanced Salt Solution (Gibco, Thermo Fisher Scientific, Waltham, Mass.) were mixed 1:1 by volume with Matrigel® (Corning, Corning, N.Y.) and subcutaneously injected into the right flank on day 0. Mice were vaccinated with 1.0×10⁶ GM-CSF-secreting B16 (GVAX) cells that had been irradiated with 3500 Gy on days 1 and 4 to elicit an anti-tumor immune response. Subsequently, mice were treated with 100 μg of rat monoclonal anti-PD1 antibody (clone: 29F.1A12) on days 9 and 12 via intraperitoneal injection. For validation assays, 1.0×10⁶ tumor cells were subcutaneously injected into the right flank without matrigel. Tumors were measured every 3 days beginning on day 6 after challenge until time of death. Measurements were taken manually by collecting the longest dimension (length) and the longest perpendicular dimension (width). Tumor volume was estimated with the formula: (L×W²)/2. C02 inhalation was used to euthanize mice on the day of sacrifice.

c. Creation of CRISPR Edited Tumor Cell Lines

Transient transfection of Cas9-sgRNA plasmid (pX459, Addgene, Cambridge, Mass.) was used to edit B16 and Braf/Pten melanoma cell lines. pX459 was digested with the enzyme Bpil (Thermo Fisher Scientific) as per the manufacturer's instructions and sgRNA oligos were cloned in using standard molecular cloning. For B16 cells, 5×10⁵ cells were plated in a well of a 6-well plate and were transfected the following day using 2 μg of pX459 plasmid DNA and Turbofect™ (3:1 ratio, Thermo Fisher Scientific). Twenty-four hours after transfection, transfectants were selected in puromycin (6 μg/mL, Thermo Fisher Scientific). For Braf/Pten melanoma cells, 5×10⁵ cells were plated in a well of a 6-well plate and were transfected the following day using 4 μg of pX459 plasmid DNA and Turbofect™ (3:1 ratio). After selection, cells were grown for 14 days in vitro before being implanted into mice.

d. In vivo competition assays

B16 cells were engineered to express GFP or TdTomato by lentiviral transduction to differentiate populations. Cas9-target sgRNA-transfected cells and Cas9-control sgRNA-transfected cells were mixed and then grown for at least two passages in vitro before implantation into mice. Mixes were analyzed by flow cytometry on the day of tumor inoculation. Mice were euthanized 15-21 days after tumor inoculation for tumor harvest. Tumors were macerated on ice and incubated in collagenase P (2 mg/mL, Sigma, St. Louis, Mo.) and DNase I (50 μg/mL, Sigma) supplemented DMEM for 10 minutes at 37° C. After incubation, tumor cells were passed through 70 m filters to remove undigested tumor. Tumor cells were washed with ice-cold MACS media and stained with Near-IR LIVE/DEAD® (1:1000, BD Biosciences, Franklin Lakes, N.J.) for 10 minutes on ice. Tumor cells were then washed and resuspended in ice-cold PBS with 2% FBS. An Accuri™ C6 flow cytometry system (BD Biosiences) was used to analyze final GFP/TdTomato tumor cell ratios.

e. Analysis Tumor-Infiltrating Lymphocytes by Flow Cytometry

Mice were injected subcutaneously (s.c.) with 1.0×10⁶ CRISPR/Cas9 modified B16 cells and treated with GVAX+anti-PD-1 mAb as described above. Tumors were harvested on day 12-13, weighed, mechanically diced, incubated with collagenase P (2 mg/mL, Sigma Aldrich) and DNAse I (50 μg/mL, Sigma Aldrich) for 10 minutes, and pipetted into a single-cell suspension. After filtering through a 70 μm filter, cells were blocked with anti-mouse CD16/32 antibody (BioLegend, San Diego, Calif.) and stained with indicated antibodies for 30 minutes on ice. Dead cells were excluded using Aqua Live/Dead® (1:1000, ThermoFisher Scientific) added concurrently with surface antibodies. After washing, cells were fixed with Foxp3/transcription factor staining buffer set (eBiosciences, San Diego, Calif.) as per manufacturer's instructions, blocked with mouse and rat serum, then stained with intracellular antibodies. AccuCount Fluorescent particles (Spherotech, Lake Forest, Ill.) were added for cell quantification prior to analysis on an LSR Fortessa™ cell analyzer (BD Biosciences) using single-color compensation controls and fluorescence-minus-one thresholds to set gate margins. Comparisons between groups performed using Student's t test.

f. Flow Cytometry Analysis of B16 Tumor Cells

B16 cells were trypsinized and washed in PBS+2% FBS, stained with antibodies for cell surface proteins as per the manufacturer's instructions and then analyzed on an Accuri™ C6 flow cytometry system (BD Biosciences).

g. RNAseq Analysis of Tumor Cells

Null or control sgRNA-transfected B16 cells were stimulated with IFNγ (100 ng/mL, Cell Signaling Technology), TNFα (10 ng/mL, Peprotech) or both for 48 hours. RNA was extracted from cell pellets using the Qiagen RNeasy Mini kit according to manufacturer's instructions. First-strand Illumina-barcoded libraries were generated using the NEB RNA Ultra™ Directional kit according to manufacturer's instructions, including a 12-cycle PCR enrichment. Libraries were sequenced on an Illumina NextSeq™ 500 instrument using paired-end 37 bp reads. Data were trimmed for quality using the Trimmomatic pipeline with the following parameters: LEADING: 15 TRAILING: 15 SLIDINGWINDOW:4:15 MINLEN: 16. Data were aligned to mouse reference genome mm10 using the Bowtie 2 aligning sequencing tool (available at the World Wide Web website of Johns Hopkins University). HTSeq was used to map aligned reads to genes and to generate a gene count matrix and it is available at the World Wide Web address of www-huber.embl.de/users/anders/HTSeq/doc/overview.html. Normalized counts and differential expression analysis was performed using the DESeq2 R package. The gene set enrichment analysis was performed as described previously in Subramanian et al. (2005) Proc Natl Acad Sci USA 102:15545-15550. Principle Components Analysis (PCA) was performed on the normalized gene counts including all genes that passed a minimal expression filter. Signature scores for the individual samples were generated using FastProject (available at the World Wide Web address of bmcbioinformatics.biomedcentral.com/articles/10.1186/s12859-016-1176-5) and the Hallmark gene signature collection (Liberzon et al. (2015) Cell Sys. 1:417-425). Pearson correlation coefficients were calculated between the Hallmark gene signatures and PC1 and PC2. Selected signatures were plotted on a normalized PCA projection of the dataset.

h. Western Blotting

Whole cell lysates were prepared in lysis buffer (60 mM Tris HCl, 2% SDS, 10% glycerol, complete EDTA-free protease-inhibitor (Roche, Basel, Switzerland), and 500 U/mL benzonase nuclease (Novagen, Merck, Darmstadt, Germany)). Samples were boiled at 100° C. and clarified by centrifugation. Protein concentration was measured with a BCA protein assay kit (Pierce, Dallas, Tex.). Fifty to one hundred and fifty micrograms of protein was loaded on 4-12% Bolt® Bis-Tris Plus gels (Life Technologies, Carlsbad, Calif.) in MES buffer (Life Technologies). Protein was transferred to 0.45 μm nitrocellulose membranes (Bio-Rad, Hercules, Calif.). Membranes were blocked in Tris-buffered saline plus 0.1% Tween 20 (TBS-T) containing 5% non-fat dry milk for 1 hour at room temperature followed by overnight incubation with primary antibody at 4° C. Membranes were washed with TBS-T and incubated with HRP-conjugated secondary antibodies for 1 hour at room temperature. HRP was activated with Supersignal® West Dura Extended Duration Substrate (Pierce) and visualized with a chemiluminscent detection system using Fuji ImageQuant™ LAS4000 (GE Healthcare Life Sciences, Pittsburgh, Pa.). Blots were then analyzed using ImageJ and Adobe® Photoshop® software.

i. Antibodies

For Western blotting, primary antibodies against P3-ACTIN (Abcam, Cambridge, UK, Cat. #8227), and FLAG (clone M2, Sigma Aldrich) were used. Peroxidase-conjugated secondaries against Rabbit-IgG (Cat. #111-035-046) and Mouse-IgG (Cat. #115-035-174) were purchased from Jackson Laboratories (Bar Harbor, Me.).

For flow cytometry, the following anti-mouse (m) fluorochrome-conjugated antibodies were used: CD47 (clone miap301, BioLegend), Granzyme B (clone GB11, BioLegend), and PD-1 (clone RPMI-30, BioLegend).

i. Crispr Sgrna Sequences.

TABLE 2

| Gene Name/sg# | sgRNA Sequence |
|---|---|
| Cd274 sgRNA 1 | GCCTGCTGTCACTTGCTACG |
| | (SEQ ID NO: 19) |
| Cd274 sgRNA 2 | AATCAACCAGAGAATTTCCG |
| | (SEQ ID NO: 20) |
| Cd274 sgRNA 3 | GGTCCAGCTCCCGTTCTACA |
| | (SEQ ID NO: 21) |
| Cd274 sgRNA 4 | GTATGGCAGCAACGTCACGA |
| | (SEQ ID NO: 22) |
| Cd47 sgRNA 1 | TATAGAGCTGAAAAACCGCA |
| | (SEQ ID NO: 23) |
| Cd47 sgRNA 2 | CCACATTACGGACGATGCAA |
| | (SEQ ID NO: 24) |
| Cd47 sgRNA 3 | TCTTACGAGGAGGAGAAAGG |
| | (SEQ ID NO: 25) |
| Cd47 sgRNA 4 | GCAAGTGTAGTTTCCCACCA |
| | (SEQ ID NO: 26) |
| control sgRNA 1 | GCGAGGTATTCGGCTCCGCG |
| | (SEQ ID NO: 27) |
| control sgRNA 2 | GCTTTCACGGAGGTTCGACG |
| | (SEQ ID NO: 28) |
| control sgRNA 3 | ATGTTGCAGTTCGGCTCGAT |
| | (SEQ ID NO: 29) |
| control sgRNA 4 | ACGTGTAAGGCGAACGCCTT |
| | (SEQ ID NO: 30) |
| control sgRNA 5 | ATTGTTCGACCGTCTACGGG |
| | (SEQ ID NO: 31) |
| Stub1 sgRNA 1 | GCATTGCTAAGAAGAAGCGC |
| | (SEQ ID NO: 32) |

Examples 2-4 disclose the development of a pooled loss-of-function in vivo genetic screening approach that uses CRISPR-Cas9 genome editing to discover genes that increase sensitivity or cause resistance to immunotherapy in a mouse transplantable tumor model. About 2,400 genes expressed by tumor cells were screened in the B16 murine melanoma model to identify those that increase or decrease sensitivity to immunotherapy with tumor vaccination and PD-1 checkpoint blockade. The screen identified known immune evasion molecules PD-L1 and CD47, as tumor cells bearing sgRNAs for these targets were significantly depleted in animals treated with immunotherapy. In contrast, loss of function of any of the genes that sense or signal in response to interferon-γ (IFNγ) rendered cells resistant to immunotherapy with PD-1 checkpoint blockade and vaccination, recapitulating resistance mutations identified in melanoma patients (Zaretsky et al. (2016) N. Engl. J. Med. 375:819-829; Gao et al. (2016) Cell 167:397-404.e9). It was discovered that deletion of one or more biomarkers listed in Table 1, such as one or more regulators of ubiquitin proteasome pathway (e.g., Stub1, Ubqln1, and Hsp90b1) significantly increased sensitivity of tumor cells to immunotherapy. These findings reveal that therapeutic strategies, such as Stub1 inhibition, that sensitize tumor cells to the effects of IFNγ are capable of increasing the efficacy of cancer immunotherapy. Moreover, this screening approach can discover new immunotherapy targets and prioritize their combination with existing immunotherapies.

### Example 2: A Pooled Loss-of-Function In Vivo Genetic Screen Recovers Known Immune Evasion Molecules Expressed by Tumors

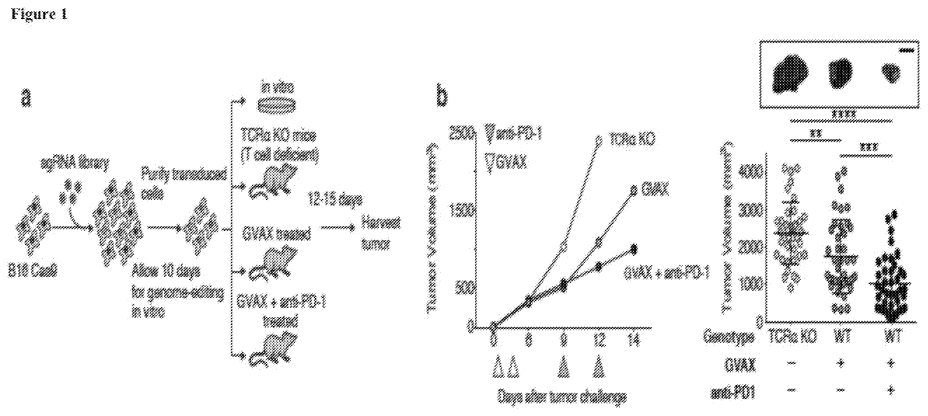

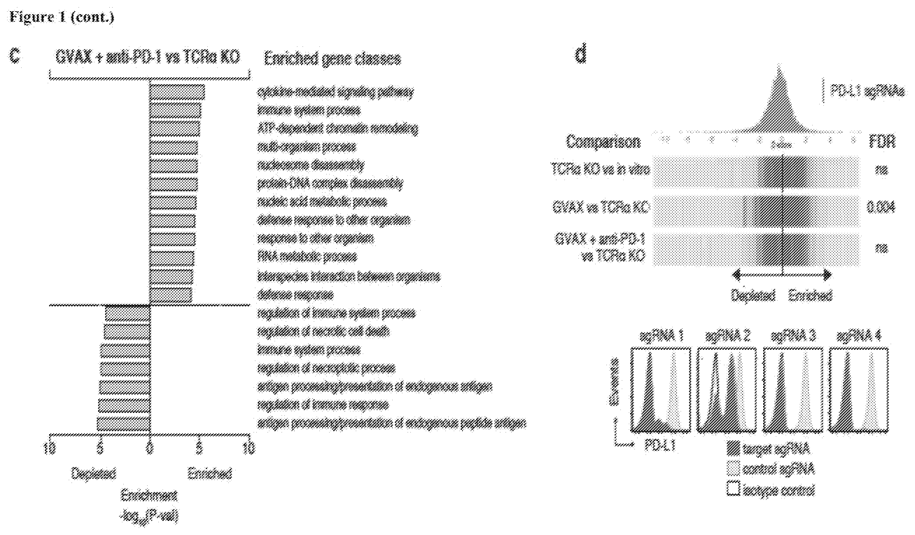

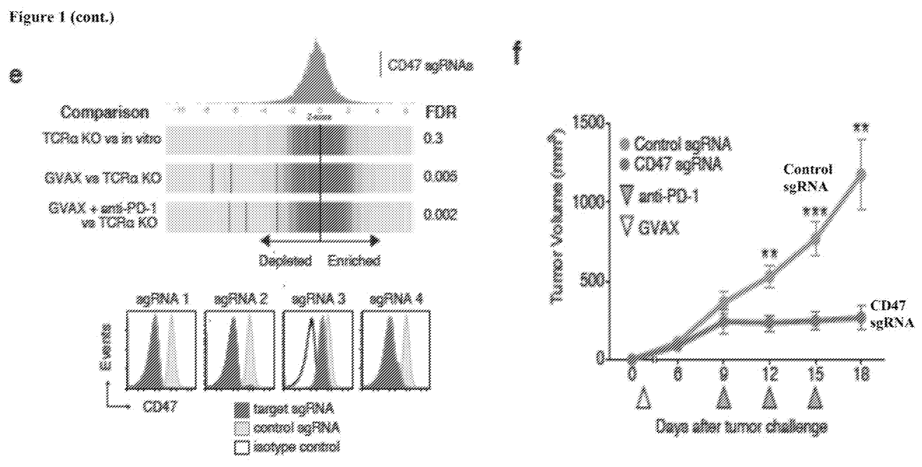

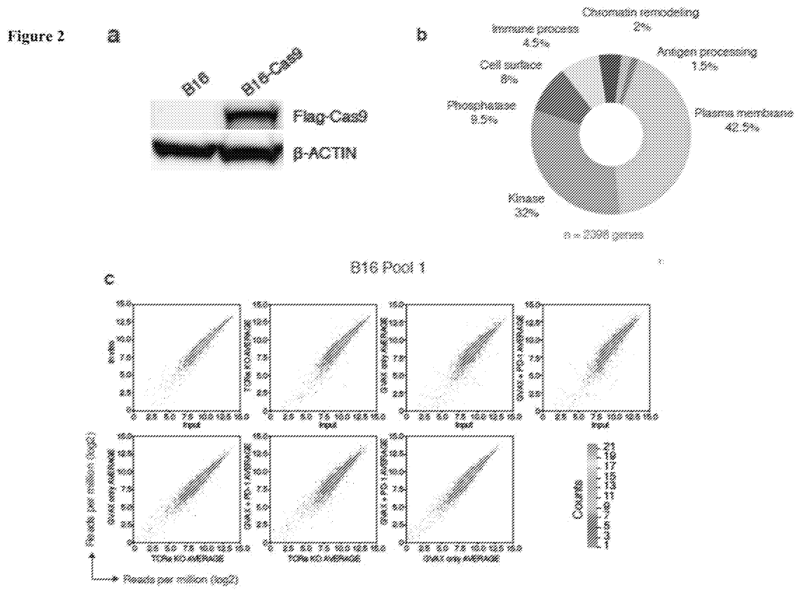

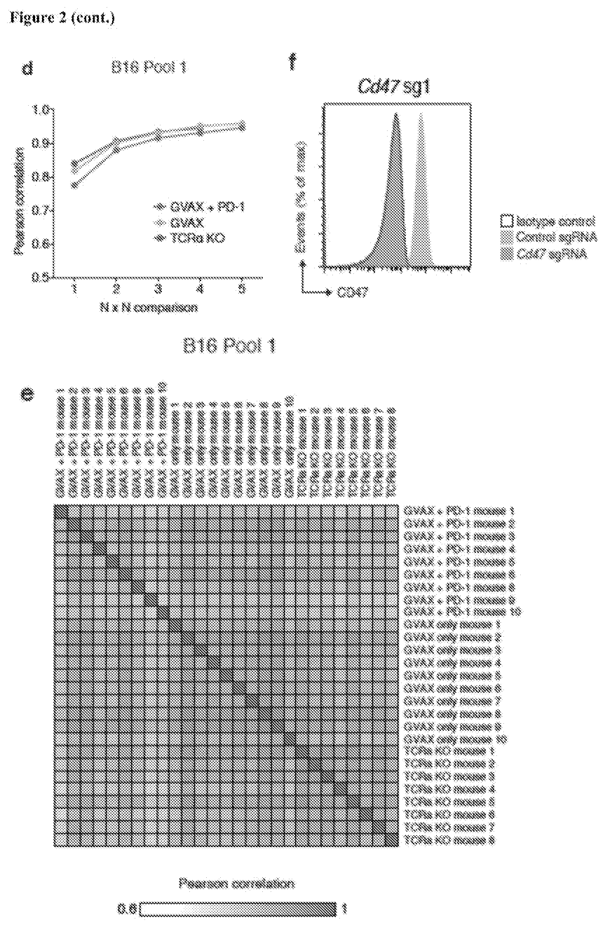

In order to systematically identify new cancer immunotherapy targets and resistance mechanisms, a pooled genetic screening approach was developed to identify genes that increase or decrease the fitness of tumor cells growing in vivo in animals treated with immunotherapy (FIG. 1A). First, a B16 melanoma cell line was engineered to express Cas9 (FIG. 2A), confirmed of efficient DNA editing using sgRNAs targeting PD-L1 (FIG. 1D). Next, a library of lentiviral vectors was created to encode 9,992 sgRNAs targeting 2,398 genes from relevant functional classes that were expressed at detectable levels in the tumor cell line (FIG. 2B). After transduction and in vitro passage to allow gene editing to take place, the tumor cells were transplanted into animals that were then treated with either a GM-CSF-secreting, irradiated tumor cell vaccine (GVAX) or GVAX plus PD-1 blockade using a monoclonal antibody for PD-1, in order to apply immune selective pressure on the tumor cells (FIG. 1B) (see Dranoff (2003) Oncogene 22:3188-3192; Dranoff et al. (1993) Proc. Natl. Acad. Sci. U.S.A. 90:3539-3543; and Duraiswamy et al. (2013) Cancer Res. 73:3591-3603). In parallel, the library-transduced tumor cells were transplanted into TCRα^{-/-} mice, which lack CD4⁺ and CD8⁺ T cells and were therefore unable to apply adaptive immune selective pressure on the tumors. This allowed to distinguish the effect of immune selective pressure on library representation from nonspecific effects on tumor cell viability. After 12-14 days, tumors were harvested (FIG. 1B), with all sgRNAs recovered from each animal with good inter-animal reproducibility (FIGS. 2C-2E).

The library representation in tumors from immunotherapy-treated wild-type (WT) animals were compared with that found in tumors growing in TCRα^{-/-} mice, in which deletion of genes that result in resistance to immunotherapy would be expected to increase tumor sgRNA representation in WT animals, while deletion of genes that result in increased sensitivity of tumors to immunotherapy would decrease sgRNA representation. Analysis of sgRNAs enriched by immune selective pressure revealed those targeting genes involved in cytokine-mediating signaling and immune-system processes (FIG. 1C). sgRNAs depleted by immunotherapy included those targeting genes involved in antigen processing, necroptosis, and regulation of immune responses (FIG. 1C). These results indicate that the genetic screening approach used here identified genes expressed by tumors cells that play a role in interaction with the immune system.

Inspection of the list of genes targeted by sgRNAs depleted from tumors treated with immunotherapy revealed the known immune evasion molecule PD-L1, indicating that loss of PD-L1 increased the sensitivity of tumor cells to immune attack. sgRNAs targeting PD-L1 were not depleted from tumors in TCRα^{-/-} mice relative to cells growing in vitro, presumably due to the absence of T cell-mediated selective pressure (FIG. 1D), but were significantly depleted in WT mice treated with GVAX relative to TCRα^{-/-} mice (FDR=0.004). However, the depletion of PD-L1-targeting sgRNAs seen in GVAX-treated tumors was not observed in tumors treated with GVAX and anti-PD-1, indicating that loss of PD-L1 does not confer a selective disadvantage to tumors when PD-L1:PD-1 interactions are blocked (FIG. 1D).

It was also found that sgRNAs targeting CD47, which enables immune evasion by impairing engulfment of tumors cells by phagocytes (as in Liu et al. (2015) Nat. Med. 21:1209-1215; Weiskopf et al. (2016) J. Clin. Invest. 126:2610-2620; and Tseng et al. (2013) Proc. Natl. Acad. Sci. U.S.A. 110: 11103-11108), were markedly depleted in tumors treated with either GVAX or with GVAX plus PD-1 blockade (FDR=0.005, 0.002 respectively) (FIG. 1E). To confirm that CD47 null tumors were more susceptible to GVAX and PD-1 blockade, CD47 null B16 melanoma cells were generated using transient transfection of a Cas9-sgRNA plasmid (as in Ran et al. (2013) Nat. Protoc. 8:2281-2308) (FIG. 2F). It was found that loss of CD47 significantly improved control of tumor growth mediated by GVAX plus anti-PD-1 immunotherapy (FIG. 1F, p<0.01).

Using the pooled loss-of-function in vivo genetic screen for identifying immune evasion molecules expressed by tumors described above, genes that increase or decrease the fitness of MC38 colon cancer cells growing in vivo in animal treated with immunotherapy.

Thus, in vivo genetic screening recovered genes known to confer tumor evasion properties on cancer cells.

### Example 3: Discovery of Novel Gene Targets to Increase the Efficacy of Immunotherapy

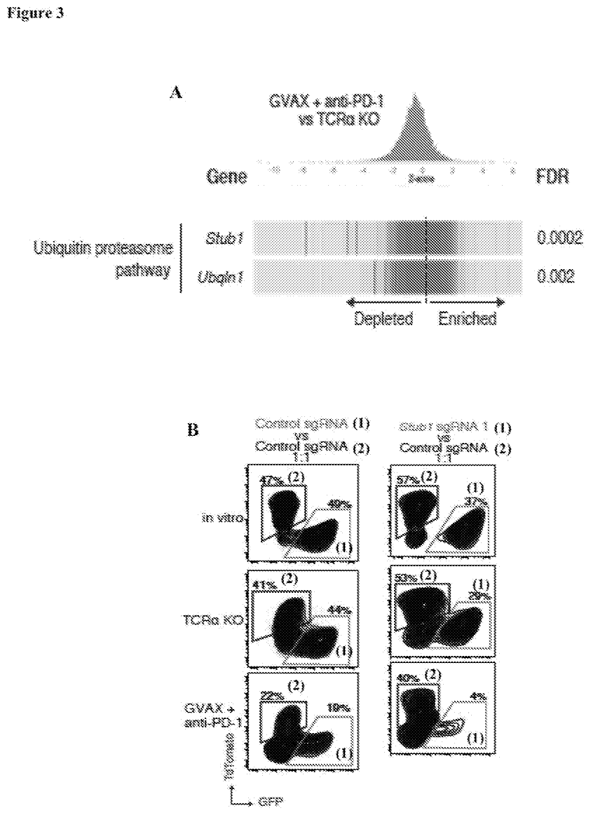

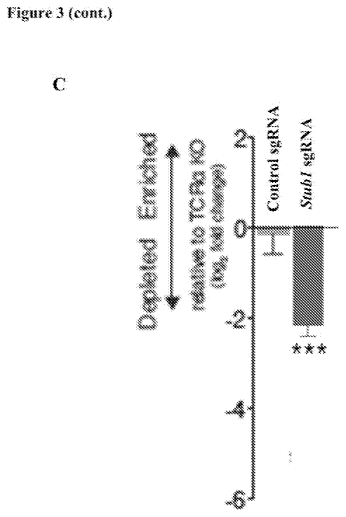

Deletion of novel candidate immunotherapy targets was found to increase sensitivity of tumor cells to immunotherapy. sgRNAs targeting genes involved in ubiquitin proteasome pathway (e.g., Stub1 and Ubqln1) were markedly depleted in mice treated with GVAX and PD-1 blockade (FIG. 3A) relative to growth in TCRα^{-/-} mice. In many cases, multiple members of the same pathway (e.g., Hsp90b1) or even the same multi-protein complex were depleted under immune selective pressure, underscoring the importance of these diverse biological pathways.

### Example 4: In Vivo Validation of STUB1 as a Target for Combination with Immunotherapy

Representative genes as regulators of ubiquitin proteasome pathway, e.g., Stub1, were selected to validate based on their highest cumulative score as ranked by the STARS algorithm (Doench et al. (2014) Nat. Biotechnol. 32:1262-1267). In vivo competition assays showed that tumor cells deleted for Stub1 were strongly selected against in WT animals treated with immunotherapy but grew at equivalent rates to control tumor cells in vitro and in TCRα^{-/-} mice (FIGS. 3B and 3C; p<0.01, Student's t test). This indicates that regulators of ubiquitin proteasome pathway, e.g., Stub1, are synthetically lethal with an anti-tumor immune response, rendering tumor cells more sensitive to immunotherapy but not altering their cell growth or survival in the absence of T cells.

One of the central challenges in cancer biology is to identify the genes that underlie the hallmarks of cancer. To date, functional genomic approaches using genome editing have largely focused on identifying genes required by tumor cells for the cancer hallmarks of growth, metastasis and drug resistance (Ebert et al. (2008) Nature 451:335-339; Hart et al. (2015) Cell 163:1515-1526; Yu et al. (2016) Nat. Biotechnol. 34:419-423; Chen et al. (2015) Cell 160:1246-1260). This study extends this approach to the interrogation of the interaction of the tumor cell with the immune system, which can be broadly applied to multiple tumor models and immunotherapy modalities to systematically define genes that govern interactions between cancer cells and the immune system.

In particular and as described above, CHIP/STUB1 is a co-chaperone/ubiquitin ligase expressed on the ER membrane that plays a crucial role in the cellular protein quality control system. STUB1 targets misfolded cytosolic proteins via ubiquination of chaperone substrates for proteasomal degradation in order to confer protection against apoptosis and cellular stress (Connell et al. (2001) Nat. Cell Biol. 3:93-96; Demand et al. (2001) Curr. Biol. 11:1569-1577; McDonough and Patterson (2003) Cell Stress Chaperones 8:303-308; Qian et al. (2006) Nature 440:551-555). In vivo screening using mouse transplantable tumor models treated with anti-PD-1 immunotherapy have shown STUB1 to be a new target that could increase the sensitivity of tumor cells to immunotherapy and synergize with PD-1 pathway blockade.

STUB1 expression on tumors has been variably associated with good and bad prognosis in different cancers (Yonezawa et al. (2017) J. Biol. Chem. doi: 10.1074/jbc.M117.785675; Shang et al. (2017) Oncogene doi: 10.1038/onc.2017.31; Sun et al. (2014) J. Cancer Res. Clin. Oncol. 140:189-197; Paul and Ghosh (2014) Biomed Res Int. 2014:918183; Esser et al. (2005) J. Bio. Chem. 280:27443-27448), making it non-obvious that inhibition of STUB1 function would have beneficial effect on the immune response to cancer. It was hypothesized that targeting STUB1 in tumor cells can lead to an increased accumulation of unfolded proteins in the cytosol, causing increased apoptosis and cellular stress. Furthermore, STUB1 has been shown to be associated with UBQLN1 and HSP90B1, which were both depleted targets in screens by the inventors, further suggesting that the inhibition of other STUB i-associated pathways could lead to increased tumor sensitivity with immunotherapy. Moreover, STUB1 is also known to be a negative regulator of the RIPK1-RIPK3 necrosome, suggesting that the knock down of STUB1 could lead to increased TNF-mediated necroptosis (Seo et al. (2016) Nat. Cell Biol. 18:291-302). These data suggest that blocking the ubiquitin ligase activity of STUB1, such as by using a small molecule, would increase the efficacy of existing immunotherapies, such as blockade of the PD-1 pathway.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a subject afflicted with a cancer comprising administering to the subject a therapeutically effective amount of an agent that inhibits the copy number, the expression level, and/or the activity of one or more biomarkers listed in Table 1 or a fragment thereof, in combination with an immunotherapy.

2. The method of claim 1, wherein the agent decreases the copy number, the expression level, and/or the activity of one or more regulators of ubiquitin proteasome pathway, preferably STIP1 homology and U-Box containing protein 1 (STUB1), ubiquilin-1 (UBQLN1), and/or heat shock protein 90 kDa beta member 1 (HSP90B1).

3. The method of claim 1 or 2, wherein the agent selectively decreases the biological activity and/or the substrate binding activity of STUB1, preferably the ubiquitin ligase activity of STUB1.

4. The method of any one of claims 1-3, wherein the agent is a small molecule inhibitor, CRISPR single-guide RNA (sgRNA), RNA interfering agent, antisense oligonucleotide, peptide or peptidomimetic inhibitor, aptamer, or intrabody.

5. The method of claim 4, wherein the RNA interfering agent is a small interfering RNA (siRNA), CRISPR RNA (crRNA), a small hairpin RNA (shRNA), a microRNA (miRNA), or a piwi-interacting RNA (piRNA).

6. The method of claim 5, wherein the RNA interfering agent is a CRISPR single-guide RNA (sgRNA).

7. The method of claim 6, wherein the sgRNA comprises a nucleic acid sequence selected from the group consisting of nucleic acid sequence listed in Table 2.

8. The method of claim 4, wherein the agent comprises an intrabody, or an antigen binding fragment thereof, which specifically binds to the one or more biomarkers and/or a substrate of the one or more biomarkers.

9. The method of claim 8, wherein the intrabody, or antigen binding fragment thereof, is murine, chimeric, humanized, composite, or human.

10. The method of claim 8 or 9, wherein the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabodies fragments.

11. The method of any one of claims 8-10, wherein the intrabody, or antigen binding fragment thereof, is conjugated to a cytotoxic agent.

12. The method of claim 11, wherein the cytotoxic agent is selected from the group consisting of a chemotherapeutic agent, a biologic agent, a toxin, and a radioactive isotope.

13. The method of any one of claims 1-12, wherein the agent increases the sensitivity of the cancer cells to an immunotherapy.

14. The method of any one of claims 1-13, wherein the immunotherapy and/or a cancer therapy is administered before, after, or concurrently with the agent.

15. The method of claim 13 or 14, wherein the immunotherapy comprises an anti-cancer vaccine and/or virus.

16. The method of any one of claims 13-15, wherein the immunotherapy is cell-based.

17. The method of any one of claims 13-16, wherein the immunotherapy inhibits an immune checkpoint.

18. The method of claim 17, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR.

19. The method of claim 18, wherein the immune checkpoint is selected from the group consisting of PD-1, PD-L1, and PD-L2, optionally wherein the immune checkpoint is PD-1.

20. The method of any one of claims 1-19, wherein the one or more biomarkers comprise a nucleic acid sequence having at least 95% identity to a nucleic acid sequence listed in Table 1 and/or an amino acid sequence having at least 95% identity to an amino acid sequence listed in Table 1.

21. The method of any one of claims 1-20, wherein the one or more biomarkers are human, mouse, chimeric, or a fusion.

22. The method of any one of claims 1-21, wherein the agent reduces the number of proliferating cells in the cancer and/or reduces the volume or size of a tumor comprising the cancer cells.

23. The method of any one of claims 1-22, wherein the one or more biomarkers comprise an amino acid sequence listed in Table 1, optionally wherein the amino acid sequence is selected from the group consisting of SEQ ID Nos: 2, 4, 6, 8, 10, 12, 14, 16, and 18.

24. The method of any one of claims 1-23, wherein the one or more biomarkers are encoded by a nucleic acid sequence listed in Table 1, optionally wherein the nucleic acid sequence is selected from the group consisting of SEQ ID Nos: 1, 3, 5, 7, 9, 11, 13, 15, and 17.

25. The method of any one of claims 1-24, wherein the cancer is melanoma.

26. The method of any one of claims 1-25, wherein the subject is an animal model of the cancer, preferably a mouse model, or a human.

27. The method of any one of claims 1-26, further comprising administering to the subject at least one additional cancer therapy or regimen, optionally wherein the at least one additional cancer therapy or regimen is administered before, after, or concurrently with the agent and/or the immunotherapy.

28. The method of any one of claims 1-27, wherein the agent is administered in a pharmaceutically acceptable formulation.

29. A method of killing cancer cells comprising contacting the cancer cells with an agent that inhibits the copy number, the expression level, and/or the activity of one or more biomarkers listed in Table 1 or a fragment thereof, in combination with an immunotherapy.

30. The method of claim 29, wherein the agent decreases the copy number, the expression level, and/or the activity of one or more regulators of ubiquitin proteasome pathway, preferably STIP1 homology and U-Box containing protein 1 (STUB1), ubiquilin-1 (UBQLN1), and/or heat shock protein 90 kDa beta member 1 (HSP90B1).

31. The method of claim 29 or 30, wherein the agent selectively decreases the biological activity and/or the substrate binding activity of STUB1, preferably the ubiquitin ligase activity of STUB1.

32. The method of any one of claims 29-31, wherein the agent is a small molecule inhibitor, CRISPR single-guide RNA (sgRNA), RNA interfering agent, antisense oligonucleotide, peptide or peptidomimetic inhibitor, aptamer, or intrabody.

33. The method of claim 32, wherein the RNA interfering agent is a small interfering RNA (siRNA), CRISPR RNA (crRNA), a small hairpin RNA (shRNA), a microRNA (miRNA), or a piwi-interacting RNA (piRNA).

34. The method of claim 33, wherein the RNA interfering agent is a CRISPR single-guide RNA (sgRNA).

35. The method of claim 34, wherein the sgRNA comprises a nucleic acid sequence selected from the group consisting of nucleic acid sequence listed in Table 2.

36. The method of claim 32, wherein the agent comprises an intrabody, or an antigen binding fragment thereof, which specifically binds to the one or more biomarkers and/or a substrate of the one or more biomarkers.

37. The method of claim 36, wherein the intrabody, or antigen binding fragment thereof,
is murine, chimeric, humanized, composite, or human.

38. The method of claim 36 or 37, wherein the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabodies fragments.

39. The method of any one of claims 36-38, wherein the intrabody, or antigen binding fragment thereof, is conjugated to a cytotoxic agent.

40. The method of claim 39, wherein the cytotoxic agent is selected from the group consisting of a chemotherapeutic agent, a biologic agent, a toxin, and a radioactive isotope.

41. The method of any one of claims 29-40, wherein the agent increases the sensitivity of the cancer cells to an immunotherapy.

42. The method of any one of claims 29-41, wherein the cancer cells are contacted with an immunotherapy and/or a cancer therapy before, after, or concurrently with the agent.

43. The method of claim 41 or 42, wherein the immunotherapy comprises an anti-cancer vaccine and/or virus.

44. The method of any one of claims 41-43, wherein the immunotherapy is cell-based.

45. The method of any one of claims 41-44, wherein the immunotherapy inhibits an immune checkpoint.

46. The method of claim 45, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR.

47. The method of claim 46, wherein the immune checkpoint is selected from the group consisting of PD-1, PD-L1, and PD-L2, optionally wherein the immune checkpoint is PD-1.

48. The method of any one of claims 29-47, wherein the one or more biomarker comprise a nucleic acid sequence having at least 95% identity to a nucleic acid sequence listed in Table 1 and/or an amino acid sequence having at least 95% identity to an amino acid sequence listed in Table 1.

49. The method of any one of claims 29-48, wherein the one or more biomarkers are human, mouse, chimeric, or a fusion.

50. The method of any one of claims 29-49, wherein the agent reduces the number of proliferating cells in the cancer and/or reduces the volume or size of a tumor comprising the cancer cells.

51. The method of any one of claims 29-50, wherein the one or more biomarkers comprise an amino acid sequence listed in Table 1, optionally wherein the amino acid sequence is selected from the group consisting of SEQ ID Nos: 2, 4, 6, 8, 10, 12, 14, 16, and 18.

52. The method of any one of claims 29-51, wherein the one or more biomarkers are encoded by a nucleic acid sequence listed in Table 1, optionally wherein the nucleic acid sequence is selected from the group consisting of SEQ ID Nos: 1, 3, 5, 7, 9, 11, 13, 15, and 17.

53. The method of any one of claims 29-52, wherein the cancer is melanoma.

54. The method of any one of claims 29-53, wherein the subject is an animal model of the cancer, preferably a mouse model, or a human.

55. The method of any one of claims 29-54, further comprising administering to the subject at least one additional cancer therapy or regimen, optionally wherein the at least one additional cancer therapy or regimen is administered before, after, or concurrently with the agent and/or the immunotherapy.

56. The method of any one of claims 29-55, wherein the agent is administered in a pharmaceutically acceptable formulation.

57. A method of determining whether a subject afflicted with a cancer or at risk for developing a cancer would benefit from inhibiting the copy number, amount, and/or activity of at least one biomarker listed in Table 1, the method comprising:
a) obtaining a biological sample from the subject;
b) determining the copy number, amount, and/or activity of at least one biomarker listed in Table 1;
c) determining the copy number, amount, and/or activity of the at least one biomarker in a control; and
d) comparing the copy number, amount, and/or activity of the at least one biomarker detected in steps b) and c);
wherein the presence of, or a significant increase in, the copy number, amount, and/or activity of, the at least one biomarker listed in Table 1 in the subject sample relative to the control copy number, amount, and/or activity of the at least one biomarker indicates that the subject afflicted with the cancer or at risk for developing the cancer would benefit from inhibiting the copy number, amount, and/or activity of the at least one biomarker listed in Table 1.

58. The method of claim 57, further comprising recommending, prescribing, or administering an agent that inhibits the at least one biomarker listed in Table 1 if the cancer is determined to benefit from the agent, optionally further administering at least one additional cancer therapy that is administered before, after, or concurrently with the agent.

59. The method of claim 57, further comprising recommending, prescribing, or administering cancer therapy other than an agent that inhibits the at least one biomarker listed in Table 1 if the cancer is determined to not benefit from the agent.

60. The method of claim 58 or 59, wherein the cancer therapy is selected from the group consisting of immunotherapy, targeted therapy, chemotherapy, radiation therapy, hormonal therapy, an anti-cancer vaccine, an anti-cancer virus, and a checkpoint inhibitor.

61. The method of any one of claims 57-60, wherein the control sample is determined from a cancerous or non-cancerous sample from either the patient or a member of the same species to which the patient belongs.

62. The method of any one of claims 57-61, wherein the control sample comprises cells.

63. A method for predicting the clinical outcome of a subject afflicted with a cancer expressing one or more biomarkers listed in Table 1 or a fragment thereof to treatment with an immunotherapy, the method comprising:
a) determining the copy number, amount, and/or activity of at least one biomarker listed in Table 1 in a subject sample;
b) determining the copy number, amount, and/or activity of the at least one biomarker in a control having a good clinical outcome; and
c) comparing the copy number, amount, and/or activity of the at least one biomarker in the subject sample and in the control;
wherein the presence of, or a significant increase in, the copy number, amount, and/or activity of, the at least one biomarker listed in Table 1 in the subject sample as compared to the copy number, amount and/or activity in the control, is an indication that the subject has a poor clinical outcome.

64. A method for monitoring the progression of a cancer in a subject, wherein the subject is administered a therapeutically effective amount of an agent that inhibits the copy number, amount, and/or activity of at least one biomarker listed in Table 1 and an immunotherapy, the method comprising:
a) detecting in a subject sample at a first point in time the copy number, amount, and/or activity of at least one biomarker listed in Table 1;
b) repeating step a) at a subsequent point in time; and
c) comparing the amount or activity of at least one biomarker listed in Table 1 detected in steps a) and b) to monitor the progression of the cancer in the subject.

65. A method of assessing the efficacy of an agent that inhibits the copy number, amount, and/or activity of at least one biomarker listed in Table 1 and an immunotherapy for treating a cancer in a subject, comprising:
a) detecting in a subject sample at a first point in time the copy number, amount, and/or or activity of at least one biomarker listed in Table 1;
b) repeating step a) during at least one subsequent point in time after administration of the agent and the immunotherapy; and
c) comparing the copy number, amount, and/or activity detected in steps a) and b), wherein the absence of, or a significant decrease in, the copy number, amount, and/or activity of, the at least one biomarker listed in Table 1, in the subsequent sample as compared to the copy number, amount, and/or activity in the sample at the first point in time, indicates that the agent and immunotherapy treats the cancer in the subject.

66. The method of claim 64 or 65, wherein between the first point in time and the subsequent point in time, the subject has undergone treatment, completed treatment, and/or is in remission for the cancer.

67. The method of claim 66, wherein the cancer treatment is selected from the group consisting of immunotherapy, targeted therapy, chemotherapy, radiation therapy, hormonal therapy, an anti-cancer vaccine, an anti-cancer virus, and a checkpoint inhibitor.

68. The method of any one of claims 64-67, wherein the first and/or at least one subsequent sample is selected from the group consisting of ex vivo and in vivo samples.

69. The method of any one of claims 64-68, wherein the first and/or at least one subsequent sample is a portion of a single sample or pooled samples obtained from the subject.

70. The method of any one of claims 57-69, wherein the sample comprises cells, serum, peritumoral tissue, and/or intratumoral tissue obtained from the subject.

71. The method of any one of claims 57-70, wherein the one or more biomarkers listed in Table 1 comprise STUB1, UBQLN1, and/or HSP90B1.

72. The method of any one of claims 57-71, wherein the cancer is melanoma.

73. The method of any one of claims 57-72, wherein the cancer is in a subject and the subject is a mammal.

74. The method of claim 73, wherein the mammal is a mouse or a human.

75. The method of claim 74, wherein the mammal is a human.


SEQUENCE LISTING (32 sequences)

SEQ ID NO: 1 - DNA, 1650 bp, Homo sapiens
acaggctgttgcgggagcgcgccctcagcgaagcaagtgaggcatctcactgggaaagtcgaatgtgtgtggcggccgccgccgaggcgggttccgaagagacctcagcagggcaggccagggcctacgcgaacgcccacccttaagagcgcggggacagggaactggagcgttcctcccagcccccgacgtcgcgggcccagtgtccccgtccaggctggttgggcgcacgcgcggccccactcgcccccacgcgtgcgtccccgctggtcccgcccccggccggaagttccggcggcggagctgggccgggcccgagcggatcgcgggctcgggctgcggggctccggctgcgggcgctgggccgcgaggcgcggagcttgggagcggagcccaggccgtgccgcgcggcgccatgaagggcaaggaggagaaggagggcggcgcacggctgggcgctggcggcggaagccccgagaagagcccgagcgcgcaggagctcaaggagcagggcaatcgtctgttcgtgggccgaaagtacccggaggcggcggcctgctacggccgcgcgatcacccggaacccgctggtggccgtgtattacaccaaccgggccttgtgctacctgaagatgcagcagcacgagcaggccctggccgactgccggcgcgccctggagctggacgggcagtctgtgaaggcgcacttcttcctggggcagtgccagctggagatggagagctatgatgaggccatcgccaatctgcagcgagcttacagcctggccaaggagcagcggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggactactgaggttccctgccctacctggcgtcctggtccaggggagccctgggcagaagcccccggcccctatacatagtttatgttcctggccaccccgaccgcttcccccaagttctgctgttggactctggactgtttcccctctcagcatcgcttttgctgggccgtgatcgtccccctttgtgggctggaaaagcaggtgagggtgggctgggctgaggccattgccgccactatctgtgtaataaaatccgtgagcacgaggtgggacgtgctggtgtgtgaaaaaaaaaaaaaaaaaaaaaaaaaaaaaaaaaaaaaaaaaaaaa

SEQ ID NO: 2 - protein, 303 aa, Homo sapiens
MKGKEEKEGGARLGAGGGSPEKSPSAQELKEQGNRLFVGRKYPEAAACYGRAITRNPLVAVYYTNRALCYLKMQQHEQALADCRRALELDGQSVKAHFFLGQCQLEMESYDEAIANLQRAYSLAKEQRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 3 - DNA, 1739 bp, Homo sapiens
acaggctgttgcgggagcgcgccctcagcgaagcaagtgaggcatctcactgggaaagtcgaatgtgtgtggcggccgccgccgaggcgggttccgaagagacctcagcagggcaggccagggcctacgcgaacgcccacccttaagagcgcggggacagggaactggagcgttcctcccagcccccgacgtcgcgggcccagtgtccccgtccaggctggttgggcgcacgcgcggccccactcgcccccacgcgtgcgtccccgctggtcccgcccccggccggaagttccggcggcggagctgggccgggcccgagcggatcgcgggctcgggctgcggggctccggctgcgggcgctgggccgcgaggcgcggagcttgggagcggagcccaggccgtgccgcgcggcgccatgaagggcaaggaggagaaggagggcggcgcacggctgggcgctggcggcggaagccccgagaagagcccgagcgcgcaggagctcaaggagcagggcaatcgtctgttcgtgggccgaaagtacccggaggcggcggcctgctacggccgcgcgatcccagagcgcagaagctgggacgggccgtgggtcagagtgggcacgctgagcctacgccctcatgcggctggcccggccttggtccctagacccggaacccgctggtggccgtgtattacaccaaccgggccttgtgctacctgaagatgcagcagcacgagcaggccctggccgactgccggcgcgccctggagctggacgggcagtctgtgaaggcgcacttcttcctggggcagtgccagctggagatggagagctatgatgaggccatcgccaatctgcagcgagcttacagcctggccaaggagcagcggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggactactgaggttccctgccctacctggcgtcctggtccaggggagccctgggcagaagcccccggcccctatacatagtttatgttcctggccaccccgaccgcttcccccaagttctgctgttggactctggactgtttcccctctcagcatcgcttttgctgggccgtgatcgtccccctttgtgggctggaaaagcaggtgagggtgggctgggctgaggccattgccgccactatctgtgtaataaaatccgtgagcacgaggtgggacgtgctggtgtgtgaaaaaaaaaaaaaaaaaaaaaaaaaaaaaaaaaaaaaaaaaaaaa

SEQ ID NO: 4 - protein, 231 aa, Homo sapiens
MQQHEQALADCRRALELDGQSVKAHFFLGQCQLEMESYDEAIANLQRAYSLAKEQRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 5 - DNA, 1672 bp, Mus musculus
ggccgcaggcagctcctcccgattcccgaggaagcccctcgagagcggtctgatctgcgggcctctcttctaccctcaattccgccttcctaccggcggtccagacgttcccaggctgtcgcgtgagcccgtgtgtgccctggtggtgcgctaggacccgggcctagtgaccggccctcggaaggccacttctcaatgaccgctgccacgttcgcccagcttggggccggagggcggggcttgagcgccgatgggcggagccttggtctgagcagggtaggtctcggttactgtagggcggggcctgtagagcgcggcggaagcttctcagagctaagggaactggctcaatccacgaggctccgcctttgctcacgtgtccccatgccagctccgccccacaccggaagttccggtggcggatcgccgaccgggcggagctgatcgctgcgcgggctgcgagatctaggtggccgggcgcggagcccaagccgtgccgcgcggcgccatgaagggcaaggaggaaaaggagggcggcgcgcggctgggcactggtggcggcggcagccctgataagagcccgagtgcgcaagagctcaaggagcagggaaaccggctcttcgtgggccgcaagtacccggaggcggcggcctgctacggccgcgccatcactcggaacccacttgtggcagtgtactacactaaccgggccctgtgctatctgaagatgcagcagcctgaacaggcacttgctgactgccggcgagccctggagctggacgggcagtctgtgaaggcgcacttcttcctggggcagtgccagctggagatggagagttatgatgaggccattgccaatctgcagcgagcctatagtttggccaaggagcagcgactcaactttggggatgatattcctagtgcccttcgcattgctaagaagaagcgctggaacagtatcgaggaacggcgcatccaccaggagagtgagctgcattcatatctcaccaggctcattgctgctgagcgagagagggaactggaggagtgtcagcggaaccacgagggtcatgaagatgatggccacatccgggcccagcaggcctgcattgaggccaagcacgataaatacatggcagatatggatgagctcttctctcaggtggacgagaaaagaaagaagcgagatatccctgactacttgtgtggcaagattagctttgagctgatgcgggaaccctgcattacacccagtggtatcacctatgaccgcaaggacattgaggagcacctgcagcgtgtgggccactttgaccctgtgacccggagccctctgacccaggaacagctcatccccaacttggccatgaaggaagtcattgacgctttcatctctgagaacggctgggtagaggactattgaggccccatgtcctgcctggcaccctggcccaggaggatctggagacggaagctccagtccctgtatagtttgtgtccctgggcctgcccccatcggccctgctgatgggttctgaactgctccccttctcagcataccccttgctggaccatgagcctcccttgtcccccttctgggctggagagtgggtgagggtgggctgaggttgctgctgctgccactgtcctgtaataaagtctgtgagcacta

SEQ ID NO: 6 - protein, 304 aa, Mus musculus
MKGKEEKEGGARLGTGGGGSPDKSPSAQELKEQGNRLFVGRKYPEAAACYGRAITRNPLVAVYYTNRALCYLKMQQPEQALADCRRALELDGQSVKAHFFLGQCQLEMESYDEAIANLQRAYSLAKEQRLNFGDDIPSALRIAKKKRWNSIEERRIHQESELHSYLTRLIAAERERELEECQRNHEGHEDDGHIRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 7 - DNA, 4177 bp, Homo sapiens
aatttcgctaaccgtgacgtcatctcggtgcgccgttacagcgcttaactggaaactgcgatttagtttttggtgaaaggagtggaaaaaaaccatgaactgacctaccccgccccattttgcttttagtccttcacactggcttcgcctgcactttgggcaatggagcgggaaaggaaaaggcacgcaggctgagctcaaagtcgcgccagccggggatgactcggcgacgccgccaggcgcgttacccggcgtgctccgcgcggcgccggcgaagggacgtgggggaaggggcagggaggaggaagcggtggctgctgcggatgtcggtgtgagcgagcggcgcctgaacacacggcggctgccgagcgcctgacccgggcctgcgccagagcctgcaccgagctccggggccccacacccgctacggtggccctgcgcccgttgctactgaggcggcgtgctctgcattcttcgctgtccaggcctgccggctctggtgtctgctggctcctccttgctcgcctgctccctcctgcttgcctgagtcaccgccgccgccgccgccacagccatggccgagagtggtgaaagcggcggtcctccgggctcccaggatagcgccgccggagccgaaggtgctggcgcccccgcggccgctgcctccgcggagcccaaaatcatgaaagtcaccgtgaagaccccgaaggaaaaggaggaattcgccgtgcccgagaatagctccgtccagcagtttaaggaagaaatctctaaacgttttaaatcacatactgaccaacttgtgttgatatttgctggaaaaattttgaaagatcaagataccttgagtcagcatggaattcatgatggacttactgttcaccttgtcattaaaacacaaaacaggcctcaggatcattcagctcagcaaacaaatacagctggaagcaatgttactacatcatcaactcctaatagtaactctacatctggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcagcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcatcccagggggatataatgctttaaggcgcatgtacacagatattcaggaaccaatgctgagtgctgcacaagagcagtttggtggtaatccatttgcttccttggtgagcaatacatcctctggtgaaggtagtcaaccttcccgtacagaaaatagagatccactacccaatccatgggctccacagacttcccagagttcatcagcttccagcggcactgccagcactgtgggtggcactactggtagtactgccagtggcacttctgggcagagtactactgcgccaaatttggtgcctggagtaggagctagtatgttcaacacaccaggaatgcagagcttgttgcaacaaataactgaaaacccacaactgatgcaaaacatgttgtctgccccctacatgagaagcatgatgcagtcactaagccagaatcctgaccttgctgcacagatgatgctgaataatcccctatttgctggaaatcctcagcttcaagaacaaatgagacaacagctcccaactttcctccaacaaatgcagaatcctgatacactatcagcaatgtcaaaccctagagcaatgcaggccttgttacagattcagcagggtttacagacattagcaacggaagccccgggcctcatcccagggtttactcctggcttgggggcattaggaagcactggaggctcttcgggaactaatggatctaacgccacacctagtgaaaacacaagtcccacagcaggaaccactgaacctggacatcagcagtttattcagcagatgctgcaggctcttgctggagtaaatcctcagctacagaatccagaagtcagatttcagcaacaactggaacaactcagtgcaatgggatttttgaaccgtgaagcaaacttgcaagctctaatagcaacaggaggtgatatcaatgcagctattgaaaggttactgggctcccagccatcatagcagcatttctgtatcttgaaaaaatgtaatttatttttgataacggctcttaaactttaaaatacctgctttatttcattttgactcttggaattctgtgctgttataaacaaacccaatatgatgcattttaaggtggagtacagtaagatgtgtgggtttttctgtatttttcttttctggaacagtgggaattaaggctactgcatgcatcacttctgcatttattgtaattttttaaaaacatcaccttttatagttgggtgaccagattttgtcctgcatctgtccagtttatttgctttttaaacattagcctatggtagtaatttatgtagaataaaagcattaaaaagaagcaaatcatttgcactctataatttgtggtacagtattgcttattgtgactttggcatgcatttttgcaaacaatgctgtaagatttatactactgataattttgttttatttgtatacaatatagagtatgcacatttgggactgcatttctggaaacatactgcaataggctctctgagcaaaacacctgtaactaaaaaagtgaagataagaaaatactcttaaagctgagtatttcctaattgtatagaatcttacagcatctttgacaaacatctcccagcaaaagtgccggttagtcaggtttgttgaaaatacagtagaaaagctgattctggttatctctttaaggacaattaattgtacagacacataatgtaacattgtctcaacattcattcacagattgactgtaaattaccttaatctttgtgcagactgaaggaacactgtagtataccccaaagtgcatttgcctaggacttctcagcttctcccataggtagtttaacaggcattaaaatttgtaattgaaatgttgctttcactgaaaaagtgtcttgatgtttcagttatttttaatcgccataaaaaaatagaactatcttttgggtttatctgttttctcatgcacaggcaatacacaaatttaaaatgagttgtgagccaattgtttctgaagtgttttggtagttctattaagaaatagttaaatattgtgcttttcagagcctcagagaaagggggacggggtgggggggtggggcagcggaatctgtcctggatggggccagcttaaataatactggcaaccaagattctgttaggatttctgtgcatatagtgtagtaaagaagtatcattcaggggtgaaaaacaaagagccgttttaatgatgttgagtacatttggctgttttatagcctttttcttccctcccccaaagaattctgtttgcctaactcccaaactgttggggtggtacattcctttaggaccaattaaaacataattgagggtcagtgatacatttggctgactctggttcagtattctcttaggtgattatattctctcatgtacagttacaggaaattaaaatgttaaagtaacctaaaatgaattcagaccaataaaatcaagggaaatacaagttgattgcattacttctgtatgttgcttgctattaaaaaggttaagaggccaggttacccaccagtccttgcactgttctgacactttccccaggaggaaaacaagtacaaaggttacggtggaggcataagtagaagagattgttaagaagggtattcatgtgtctttgctctttctgctttatgcctcagtttggtttaaaaacttctgtactggcaaatggtggtattcagtgtgggatagtgtcataactaatttgacaatttattaatcataaaataacaataaatctctagcttttacacttgaaaaaaaaaa

SEQ ID NO: 8 - protein, 589 aa, Homo sapiens
MAESGESGGPPGSQDSAAGAEGAGAPAAAASAEPKIMKVTVKTPKEKEEFAVPENSSVQQFKEEISKRFKSHTDQLVLIFAGKILKDQDTLSQHGIHDGLTVHLVIKTQNRPQDHSAQQTNTAGSNVTTSSTPNSNSTSGSATSNPFGLGGLGGLAGLSSLGLNTTNFSELQSQMQRQLLSNPEMMVQIMENPFVQSMLSNPDLMRQLIMANPQMQQLIQRNPEISHMLNNPDIMRQTLELARNPAMMQEMMRNQDRALSNLESIPGGYNALRRMYTDIQEPMLSAAQEQFGGNPFASLVSNTSSGEGSQPSRTENRDPLPNPWAPQTSQSSSASSGTASTVGGTTGSTASGTSGQSTTAPNLVPGVGASMFNTPGMQSLLQQITENPQLMQNMLSAPYMRSMMQSLSQNPDLAAQMMLNNPLFAGNPQLQEQMRQQLPTFLQQMQNPDTLSAMSNPRAMQALLQIQQGLQTLATEAPGLIPGFTPGLGALGSTGGSSGTNGSNATPSENTSPTAGTTEPGHQQFIQQMLQALAGVNPQLQNPEVRFQQQLEQLSAMGFLNREANLQALIATGGDINAAIERLLGSQPS

SEQ ID NO: 9 - DNA, 4093 bp, Homo sapiens
aatttcgctaaccgtgacgtcatctcggtgcgccgttacagcgcttaactggaaactgcgatttagtttttggtgaaaggagtggaaaaaaaccatgaactgacctaccccgccccattttgcttttagtccttcacactggcttcgcctgcactttgggcaatggagcgggaaaggaaaaggcacgcaggctgagctcaaagtcgcgccagccggggatgactcggcgacgccgccaggcgcgttacccggcgtgctccgcgcggcgccggcgaagggacgtgggggaaggggcagggaggaggaagcggtggctgctgcggatgtcggtgtgagcgagcggcgcctgaacacacggcggctgccgagcgcctgacccgggcctgcgccagagcctgcaccgagctccggggccccacacccgctacggtggccctgcgcccgttgctactgaggcggcgtgctctgcattcttcgctgtccaggcctgccggctctggtgtctgctggctcctccttgctcgcctgctccctcctgcttgcctgagtcaccgccgccgccgccgccacagccatggccgagagtggtgaaagcggcggtcctccgggctcccaggatagcgccgccggagccgaaggtgctggcgcccccgcggccgctgcctccgcggagcccaaaatcatgaaagtcaccgtgaagaccccgaaggaaaaggaggaattcgccgtgcccgagaatagctccgtccagcagtttaaggaagaaatctctaaacgttttaaatcacatactgaccaacttgtgttgatatttgctggaaaaattttgaaagatcaagataccttgagtcagcatggaattcatgatggacttactgttcaccttgtcattaaaacacaaaacaggcctcaggatcattcagctcagcaaacaaatacagctggaagcaatgttactacatcatcaactcctaatagtaactctacatctggttctgctactagcaacccttttggtttaggtggccttgggggacttgcaggtctgagtagcttgggtttgaatactaccaacttctctgaactacagagtcagatgcagcgacaacttttgtctaaccctgaaatgatggtccagatcatggaaaatccctttgttcagagcatgctctcaaatcctgacctgatgagacagttaattatggccaatccacaaatgcagcagttgatacagagaaatccagaaattagtcatatgttgaataatccagatataatgagacaaacgttggaacttgccaggaatccagcaatgatgcaggagatgatgaggaaccaggaccgagctttgagcaacctagaaagcatcccagggggatataatgctttaaggcgcatgtacacagatattcaggaaccaatgctgagtgctgcacaagagcagtttggtggtaatccatttgcttccttggtgagcaatacatcctctggtgaaggtagtcaaccttcccgtacagaaaatagagatccactacccaatccatgggctccacagacttcccagagttcatcagcttccagcggcactgccagcactgtgggtggcactactggtagtactgccagtggcacttctgggcagagtactactgcgccaaatttggtgcctggagtaggagctagtatgttcaacacaccaggaatgcagagcttgttgcaacaaataactgaaaacccacaactgatgcaaaacatgttgtctgccccctacatgagaagcatgatgcagtcactaagccagaatcctgaccttgctgcacagatgcagaatcctgatacactatcagcaatgtcaaaccctagagcaatgcaggccttgttacagattcagcagggtttacagacattagcaacggaagccccgggcctcatcccagggtttactcctggcttgggggcattaggaagcactggaggctcttcgggaactaatggatctaacgccacacctagtgaaaacacaagtcccacagcaggaaccactgaacctggacatcagcagtttattcagcagatgctgcaggctcttgctggagtaaatcctcagctacagaatccagaagtcagatttcagcaacaactggaacaactcagtgcaatgggatttttgaaccgtgaagcaaacttgcaagctctaatagcaacaggaggtgatatcaatgcagctattgaaaggttactgggctcccagccatcatagcagcatttctgtatcttgaaaaaatgtaatttatttttgataacggctcttaaactttaaaatacctgctttatttcattttgactcttggaattctgtgctgttataaacaaacccaatatgatgcattttaaggtggagtacagtaagatgtgtgggtttttctgtatttttcttttctggaacagtgggaattaaggctactgcatgcatcacttctgcatttattgtaattttttaaaaacatcaccttttatagttgggtgaccagattttgtcctgcatctgtccagtttatttgctttttaaacattagcctatggtagtaatttatgtagaataaaagcattaaaaagaagcaaatcatttgcactctataatttgtggtacagtattgcttattgtgactttggcatgcatttttgcaaacaatgctgtaagatttatactactgataattttgttttatttgtatacaatatagagtatgcacatttgggactgcatttctggaaacatactgcaataggctctctgagcaaaacacctgtaactaaaaaagtgaagataagaaaatactcttaaagctgagtatttcctaattgtatagaatcttacagcatctttgacaaacatctcccagcaaaagtgccggttagtcaggtttgttgaaaatacagtagaaaagctgattctggttatctctttaaggacaattaattgtacagacacataatgtaacattgtctcaacattcattcacagattgactgtaaattaccttaatctttgtgcagactgaaggaacactgtagtataccccaaagtgcatttgcctaggacttctcagcttctcccataggtagtttaacaggcattaaaatttgtaattgaaatgttgctttcactgaaaaagtgtcttgatgtttcagttatttttaatcgccataaaaaaatagaactatcttttgggtttatctgttttctcatgcacaggcaatacacaaatttaaaatgagttgtgagccaattgtttctgaagtgttttggtagttctattaagaaatagttaaatattgtgcttttcagagcctcagagaaagggggacggggtgggggggtggggcagcggaatctgtcctggatggggccagcttaaataatactggcaaccaagattctgttaggatttctgtgcatatagtgtagtaaagaagtatcattcaggggtgaaaaacaaagagccgttttaatgatgttgagtacatttggctgttttatagcctttttcttccctcccccaaagaattctgtttgcctaactcccaaactgttggggtggtacattcctttaggaccaattaaaacataattgagggtcagtgatacatttggctgactctggttcagtattctcttaggtgattatattctctcatgtacagttacaggaaattaaaatgttaaagtaacctaaaatgaattcagaccaataaaatcaagggaaatacaagttgattgcattacttctgtatgttgcttgctattaaaaaggttaagaggccaggttacccaccagtccttgcactgttctgacactttccccaggaggaaaacaagtacaaaggttacggtggaggcataagtagaagagattgttaagaagggtattcatgtgtctttgctctttctgctttatgcctcagtttggtttaaaaacttctgtactggcaaatggtggtattcagtgtgggatagtgtcataactaatttgacaatttattaatcataaaataacaataaatctctagcttttacacttgaaaaaaaaaa

SEQ ID NO: 10 - protein, 561 aa, Homo sapiens
MAESGESGGPPGSQDSAAGAEGAGAPAAAASAEPKIMKVTVKTPKEKEEFAVPENSSVQQFKEEISKRFKSHTDQLVLIFAGKILKDQDTLSQHGIHDGLTVHLVIKTQNRPQDHSAQQTNTAGSNVTTSSTPNSNSTSGSATSNPFGLGGLGGLAGLSSLGLNTTNFSELQSQMQRQLLSNPEMMVQIMENPFVQSMLSNPDLMRQLIMANPQMQQLIQRNPEISHMLNNPDIMRQTLELARNPAMMQEMMRNQDRALSNLESIPGGYNALRRMYTDIQEPMLSAAQEQFGGNPFASLVSNTSSGEGSQPSRTENRDPLPNPWAPQTSQSSSASSGTASTVGGTTGSTASGTSGQSTTAPNLVPGVGASMFNTPGMQSLLQQITENPQLMQNMLSAPYMRSMMQSLSQNPDLAAQMQNPDTLSAMSNPRAMQALLQIQQGLQTLATEAPGLIPGFTPGLGALGSTGGSSGTNGSNATPSENTSPTAGTTEPGHQQFIQQMLQALAGVNPQLQNPEVRFQQQLEQLSAMGFLNREANLQALIATGGDINAAIERLLGSQPS

SEQ ID NO: 11 - DNA, 3693 bp, Mus musculus
agagtgccctgcgcggcgccggcgacgagcgtgggggaggtgcgggaggaggaagcggcggcgacgcggagagtccccgcgtccacacggcagccgatcgcccacccggggcctgcacccgcagcccgcgtccacgcaccctcggcggccccgcagcctgtcacgcctcgcggacccggccgcagcccgcagtgtcgcgctcgcctgagtcaccgccatggccgagagcgcagagagcggcggcccaccgggcgcgcaggacagtgcggccgacggcggccccgcagagcccaagatcatgaaggtcacggtgaagacgcctaaagagaaggaggagtttgccgtgcccgagaacagctcggtccagcagttcaaagaggaaatctctaaacgtttcaaatctcatattgaccaacttgtgttgatatttgctggaaaaattttaaaagatcaagatactctgagtcagcatgggattcatgatggacttacggttcaccttgtcatcaaaacacagaacaggccgcaagataattcagctcagcaaacaaatgcccctggaagcactgtgaccagctcaccagctcccgatagcaaccccacgtctggttctgctgctaacagctcctttggcgtaggcggacttggaggacttgcaggtcttagcagcttgggtttaaacaccaccaacttctctgaactccagagccaaatgcagcggcaacttttgtctaaccctgaaatgatggtccagatcatggaaaatccctttgtccagagcatgctctcaaatcctgacctgatgcggcaattgattatggccaatccacagatgcaacagttgatacagagaaatccagagattagtcatatgcttaataatccagatataatgagacaaacactggaacttgcgagaaatccagcgatgatgcaggaaatgatgagaaaccaggaccgagccttgagcaacctagaaagtattcctgggggctataatgccttacggcgcatgtacaccgatatccaggagcctatgctgaatgctgctcaagaacagtttggtggtaatccatttgcgtccttagtgagcagttcgtcctcagcagaagggactcagccttctcgaacagaaaatagggatccgctccccaacccgtgggctccgcagacttcccagagctcaccagcctccggtaccactggcagcaccactaacaccatgagcacctctggtggcactgccaccagtacccctgctggacagagcacctcgggcccaagcttggtgcctggcgcaggagctagtatgttcaacacccccggaatgcagagcctgctgcagcagataactgagaacccacagctcatgcagaacatgctgtccgccccatacatgaggagcatgctgcagtccctgagccagaaccccgaccttgctgcgcagatgatgctgaataatcccctatttgctggaaatcctcagcttcaagaacaaatgagacaacagctcccaactttcctccaacaaatgcagaaccccgacacgctctcagcgatgtcgaatcctagagccatgcaggcgctgctgcagatccagcagggcttgcagacgctggccacagaagctcctggcctcatcccagggtttactcctggcttggcagcaggcaattctggaggctcttccggaaccaatgcacctagcactgcacctagtgaagatacgaacccccaagggggcactgctgagccaggccaccagcagtttatccagcagatgctgcaggcccttgcgggggtgaatcctcagctgcagagtccagaagtcagatttcagcaacaactggaacaactcagtgccatgggattcttgaaccgtgaagcaaacctgcaagctctgatagccacagggggcgacatcaatgcagcaatcgaaaggttgctgggctcccagccgtcatagcagcatctctctatcctgagaaaatgtaatttatttttgataacggctcttaaatctttaaaataacatgctttatttcattgggattctgtgctgttataaaggaaaccaataggaagtatctggaggcagagtgcagtacgacgggcgggtttctgtgtctttttggaacagtgggaatcaatgtttcttctggttaaggctgctgcatgcatcaaacacttggcatttattgtaatttttaaaaaaatatcaccttttctaattgggtgaacaaatttttgtcctgcatctgtccaactaacttgctttttaaacataaccctatggtagtaatttatgtagaataaaggcattaaaaagcagatcatttgcgcgccccatgatttctggtacaatagtgcttgttgtgactttggcatgcactttgcaaatgatgctgtaagatttatactcctgacaattttgtttatttgtatacaatacaaattattgcacatttctaggccatctaagcaaaatttctgtaactaaaaaagtacaataaggattcctaattgtgtagaatcttacagcatctttgataaacatctcagcagaagtgccatttagtctgtgtggaaaataaagaaaaactaactctggttatctctttagggacatttaattgtacagacaacataatgtaacaatatctcagcattatataatggctgtaaattacctttccttgtgccgactggaggagcactgtatcatgctctgatgtgtttgctggggcgtcctgctccccgtaggtgtcgtctacgcagcattgagtttattgaaatgtcgctttcactggaagtgcggatgtttcggttctaattgccatataaaagtagagctgtctcttgggtttatcagctttctcattcataggcaatacctcgattttaaatgattagtgagccacttgtttctcaagtgtttggctagttctattaagaaatagttgaatgtcgtgctgttcggagcctcagaagggggtgggggaggggtgaaacagaatctattctggattgggccagcctgaactaggtaatactggcaattaagattctgtgaagattctatgcatatagtagtgtcatttgggtgaaaaagagccgttttaacaattttgagtacgtttggctattctatagcctctttcttccttccccaaagacgttctacttgcctaactctcaaactgttggggtgatacattcctttgggaccagttaaaatacagcgttagggtcggtgacatatgtgactggctctggttcagtattctcttatcttaggtcactgtagtctctcttgtatggttgcccaaaattataatgttaaataatctaaagtgagttcagacaagtaaaatcaagggaaatagaaggtgaattctataacttctgtaagttgcttgatattaaaaggttgagaggcaggttggccccagactgtatacttttctgacactttacctgggaagaaaacatgtacaaaggtcagggtggaggcatgactgaggaggtcggcacagcctgcatgtgtgtgctctctctgccttacgcctcagtctgtggttggaagactttgtactggtgaatggcaatatgcaggtggggcagtgtcaaaagcagtttgatttattaatctccaaataacaataaatctccagcttttacacttgaaaaaaa

SEQ ID NO: 12 - protein, 582 aa, Mus musculus
MAESAESGGPPGAQDSAADGGPAEPKIMKVTVKTPKEKEEFAVPENSSVQQFKEEISKRFKSHIDQLVLIFAGKILKDQDTLSQHGIHDGLTVHLVIKTQNRPQDNSAQQTNAPGSTVTSSPAPDSNPTSGSAANSSFGVGGLGGLAGLSSLGLNTTNFSELQSQMQRQLLSNPEMMVQIMENPFVQSMLSNPDLMRQLIMANPQMQQLIQRNPEISHMLNNPDIMRQTLELARNPAMMQEMMRNQDRALSNLESIPGGYNALRRMYTDIQEPMLNAAQEQFGGNPFASLVSSSSSAEGTQPSRTENRDPLPNPWAPQTSQSSPASGTTGSTTNTMSTSGGTATSTPAGQSTSGPSLVPGAGASMFNTPGMQSLLQQITENPQLMQNMLSAPYMRSMLQSLSQNPDLAAQMMLNNPLFAGNPQLQEQMRQQLPTFLQQMQNPDTLSAMSNPRAMQALLQIQQGLQTLATEAPGLIPGFTPGLAAGNSGGSSGTNAPSTAPSEDTNPQGGTAEPGHQQFIQQMLQALAGVNPQLQSPEVRFQQQLEQLSAMGFLNREANLQALIATGGDINAAIERLLGSQPS

SEQ ID NO: 13 - DNA, 3609 bp, Mus musculus
agagtgccctgcgcggcgccggcgacgagcgtgggggaggtgcgggaggaggaagcggcggcgacgcggagagtccccgcgtccacacggcagccgatcgcccacccggggcctgcacccgcagcccgcgtccacgcaccctcggcggccccgcagcctgtcacgcctcgcggacccggccgcagcccgcagtgtcgcgctcgcctgagtcaccgccatggccgagagcgcagagagcggcggcccaccgggcgcgcaggacagtgcggccgacggcggccccgcagagcccaagatcatgaaggtcacggtgaagacgcctaaagagaaggaggagtttgccgtgcccgagaacagctcggtccagcagttcaaagaggaaatctctaaacgtttcaaatctcatattgaccaacttgtgttgatatttgctggaaaaattttaaaagatcaagatactctgagtcagcatgggattcatgatggacttacggttcaccttgtcatcaaaacacagaacaggccgcaagataattcagctcagcaaacaaatgcccctggaagcactgtgaccagctcaccagctcccgatagcaaccccacgtctggttctgctgctaacagctcctttggcgtaggcggacttggaggacttgcaggtcttagcagcttgggtttaaacaccaccaacttctctgaactccagagccaaatgcagcggcaacttttgtctaaccctgaaatgatggtccagatcatggaaaatccctttgtccagagcatgctctcaaatcctgacctgatgcggcaattgattatggccaatccacagatgcaacagttgatacagagaaatccagagattagtcatatgcttaataatccagatataatgagacaaacactggaacttgcgagaaatccagcgatgatgcaggaaatgatgagaaaccaggaccgagccttgagcaacctagaaagtattcctgggggctataatgccttacggcgcatgtacaccgatatccaggagcctatgctgaatgctgctcaagaacagtttggtggtaatccatttgcgtccttagtgagcagttcgtcctcagcagaagggactcagccttctcgaacagaaaatagggatccgctccccaacccgtgggctccgcagacttcccagagctcaccagcctccggtaccactggcagcaccactaacaccatgagcacctctggtggcactgccaccagtacccctgctggacagagcacctcgggcccaagcttggtgcctggcgcaggagctagtatgttcaacacccccggaatgcagagcctgctgcagcagataactgagaacccacagctcatgcagaacatgctgtccgccccatacatgaggagcatgctgcagtccctgagccagaaccccgaccttgctgcgcagatgcagaaccccgacacgctctcagcgatgtcgaatcctagagccatgcaggcgctgctgcagatccagcagggcttgcagacgctggccacagaagctcctggcctcatcccagggtttactcctggcttggcagcaggcaattctggaggctcttccggaaccaatgcacctagcactgcacctagtgaagatacgaacccccaagggggcactgctgagccaggccaccagcagtttatccagcagatgctgcaggcccttgcgggggtgaatcctcagctgcagagtccagaagtcagatttcagcaacaactggaacaactcagtgccatgggattcttgaaccgtgaagcaaacctgcaagctctgatagccacagggggcgacatcaatgcagcaatcgaaaggttgctgggctcccagccgtcatagcagcatctctctatcctgagaaaatgtaatttatttttgataacggctcttaaatctttaaaataacatgctttatttcattgggattctgtgctgttataaaggaaaccaataggaagtatctggaggcagagtgcagtacgacgggcgggtttctgtgtctttttggaacagtgggaatcaatgtttcttctggttaaggctgctgcatgcatcaaacacttggcatttattgtaatttttaaaaaaatatcaccttttctaattgggtgaacaaatttttgtcctgcatctgtccaactaacttgctttttaaacataaccctatggtagtaatttatgtagaataaaggcattaaaaagcagatcatttgcgcgccccatgatttctggtacaatagtgcttgttgtgactttggcatgcactttgcaaatgatgctgtaagatttatactcctgacaattttgtttatttgtatacaatacaaattattgcacatttctaggccatctaagcaaaatttctgtaactaaaaaagtacaataaggattcctaattgtgtagaatcttacagcatctttgataaacatctcagcagaagtgccatttagtctgtgtggaaaataaagaaaaactaactctggttatctctttagggacatttaattgtacagacaacataatgtaacaatatctcagcattatataatggctgtaaattacctttccttgtgccgactggaggagcactgtatcatgctctgatgtgtttgctggggcgtcctgctccccgtaggtgtcgtctacgcagcattgagtttattgaaatgtcgctttcactggaagtgcggatgtttcggttctaattgccatataaaagtagagctgtctcttgggtttatcagctttctcattcataggcaatacctcgattttaaatgattagtgagccacttgtttctcaagtgtttggctagttctattaagaaatagttgaatgtcgtgctgttcggagcctcagaagggggtgggggaggggtgaaacagaatctattctggattgggccagcctgaactaggtaatactggcaattaagattctgtgaagattctatgcatatagtagtgtcatttgggtgaaaaagagccgttttaacaattttgagtacgtttggctattctatagcctctttcttccttccccaaagacgttctacttgcctaactctcaaactgttggggtgatacattcctttgggaccagttaaaatacagcgttagggtcggtgacatatgtgactggctctggttcagtattctcttatcttaggtcactgtagtctctcttgtatggttgcccaaaattataatgttaaataatctaaagtgagttcagacaagtaaaatcaagggaaatagaaggtgaattctataacttctgtaagttgcttgatattaaaaggttgagaggcaggttggccccagactgtatacttttctgacactttacctgggaagaaaacatgtacaaaggtcagggtggaggcatgactgaggaggtcggcacagcctgcatgtgtgtgctctctctgccttacgcctcagtctgtggttggaagactttgtactggtgaatggcaatatgcaggtggggcagtgtcaaaagcagtttgatttattaatctccaaataacaataaatctccagcttttacacttgaaaaaaa

SEQ ID NO: 14 - protein, 554 aa, Mus musculus
MAESAESGGPPGAQDSAADGGPAEPKIMKVTVKTPKEKEEFAVPENSSVQQFKEEISKRFKSHIDQLVLIFAGKILKDQDTLSQHGIHDGLTVHLVIKTQNRPQDNSAQQTNAPGSTVTSSPAPDSNPTSGSAANSSFGVGGLGGLAGLSSLGLNTTNFSELQSQMQRQLLSNPEMMVQIMENPFVQSMLSNPDLMRQLIMANPQMQQLIQRNPEISHMLNNPDIMRQTLELARNPAMMQEMMRNQDRALSNLESIPGGYNALRRMYTDIQEPMLNAAQEQFGGNPFASLVSSSSSAEGTQPSRTENRDPLPNPWAPQTSQSSPASGTTGSTTNTMSTSGGTATSTPAGQSTSGPSLVPGAGASMFNTPGMQSLLQQITENPQLMQNMLSAPYMRSMLQSLSQNPDLAAQMQNPDTLSAMSNPRAMQALLQIQQGLQTLATEAPGLIPGFTPGLAAGNSGGSSGTNAPSTAPSEDTNPQGGTAEPGHQQFIQQMLQALAGVNPQLQSPEVRFQQQLEQLSAMGFLNREANLQALIATGGDINAAIERLLGSQPS

SEQ ID NO: 15 - DNA, 2879 bp, Homo sapiens
gattggtgggttcatgtttcccgtcccccgcccgcgggaagtgggggtgaaaagcggcccgacctgcttgcggtgtagtgggcggaccgcgcggctggaggtgtgaggatccgaacccaggggtggggggtggaggcggctcctgcgatcgaaggggacttgagactcaccggccgcacgccatgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaaaagatgaattgtaaattatactctcaccatttggatcctgtgtggagagggaatgtgaaatttacatcatttctttttgggagagacttgttttggatgccccctaatccccttctcccctgcactgtaaaatgtgggattatgggtcacaggaaaaagtgggttttttagttgaattttttttaacattcctcatgaatgtaaatttgtactatttaactgactattcttgatgtaaaatcttgtcatgtgtataaaaataaaaaagatcccaaatactcaaaaaaaaaaaaaaaaaa

SEQ ID NO: 16 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 17 - DNA, 2759 bp, Mus musculus
tggaggtgtgaggagcttagactcgggattgggggggtggaggcggctcctgagaccgaaaaggacttgcgactcgccggccacgcaccatgagggtcctgtgggtgttgggcctctgctgtgtcctgctgaccttcgggttcgtcagagctgatgatgaagtcgacgtggatggcacagtggaagaggacctgggtaaaagccgagaaggctcaaggacagatgatgaagttgtgcagagagaggaagaagctattcagttggatgggttaaacgcatcacagataagagaacttagagaaaaatctgaaaagttcgccttccaagctgaagtgaacaggatgatgaaacttatcatcaattctttgtataaaaataaagagattttcctgagagaactgatttcaaatgcttctgatgctttagacaagataaggctcatctccctaactgatgaaaatgcactcgctggaaatgaggagttaacggtcaagattaagtgtgacaaagagaaaaacctgctgcatgtcacagacacgggtgtaggaatgactagagaggagttggttaaaaatctcggcaccatagccaaatctggaacaagcgagtttttaaacaaaatgacagaagctcaagaagatggtcagtcaacctctgaactgattggccagtttggtgtcggtttttattctgccttccttgtagcagataaggtcattgtcacatcgaaacacaacaatgatacccagcacatctgggaatcagactccaatgaattctctgtaattgctgacccaagaggaaacacactaggtcgtggaacaacaattactcttgtcttaaaagaagaagcatctgattaccttgaattggacacaattaaaaatcttgtcaggaagtactctcagttcatcaactttcccatctacgtgtggagtagcaagacagagactgttgaggagcccttggaagaagatgaagcagcaaaagaagagaaagaagaatctgatgatgaagctgcagtagaggaggaagaagaagaaaagaaaccaaaaactaagaaagttgaaaaaactgtgtgggattgggaacttatgaatgatatcaaaccaatatggcagagaccatccaaagaagtagaagaagacgaatacaaagctttctacaaatcattttcaaaggaaagtgatgaccccatggcttatatccacttcactgcagaaggggaggtcaccttcaagtcgattttgtttgtacccacatctgcacctcgaggtctgtttgatgaatatggatctaagaagagtgattatattaagctgtatgtacgccgcgtattcatcacagatgacttccatgatatgatgcccaaataccttaattttgtcaaaggtgttgtggattccgatgatctccccctcaatgtttcccgtgagactcttcagcaacataaattgctcaaggtgattaggaagaagcttgtccgaaaaactctggacatgatcaagaagattgctgatgagaagtataacgacactttctggaaggagttcggcacgaatatcaagcttggtgtgattgaagaccactcaaatcgaacacggcttgctaaacttcttaggttccagtcttctcaccattcaactgacattactagtttagaccagtatgtggaaagaatgaaggaaaaacaggacaaaatctacttcatggctgggtcaagcagaaaggaggcggaatcttctccatttgttgagagacttctgaagaagggctatgaagtcatttatctcacagagcctgtggatgaatactgcattcaggctcttcccgagtttgatgggaagaggtttcagaatgttgccaaagaaggggtgaagtttgatgagagtgagaaaactaaagaaagtcgggaagcaacagagaaggagtttgaacctctgctcaactggatgaaagataaggccctcaaggacaagatagaaaaggctgtggtgtcgcagcgcctcacagagtctccctgtgctcttgtggccagtcagtatggatggtctggcaacatggagaggatcatgaaggcacaagcataccagacgggcaaggacatctctacaaattactatgccagtcaaaagaaaacgttcgaaatcaatcctagacacccactgatcagagacatgttgcggcggattaaggaagatgaagatgacaagacagtcatggatcttgctgtagttttgtttgaaacggcaacacttcggtcaggatatcttctaccagacaccaaggcgtatggagatagaatagaaagaatgcttcgcctcagtttaaacattgaccctgaagcacaggtggaggaagaaccagaagaagagcctgaagacacctcagaagacgcagaagactcagagcaggatgagggagaagagatggatgcagggacagaagaagaagaggaggaaacagaaaaggaatctacagagaaggatgaattgtaaattatactctcgctatgaatcccgtgtggagagggaatgtgaagttttgaagtcatttcttttgagagacttgttttggatgcttccccaagcctccttctcccctgcactgtaaaatgttgggattatgggtcacaggaagaagtggtttttttagttgaattttttttaacattcctcctgaatgtaaatttgtactatttaactgactattggtgtaaaatcttgtcatgtgtataaaaataaaaaaagatcccaaat

SEQ ID NO: 18 - protein, 802 aa, Mus musculus
MRVLWVLGLCCVLLTFGFVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALAGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVRKYSQFINFPIYVWSSKTETVEEPLEEDEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHSTDITSLDQYVERMKEKQDKIYFMAGSSRKEAESSPFVERLLKKGYEVIYLTEPVDEYCIQALPEFDGKRFQNVAKEGVKFDESEKTKESREATEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVMDLAVVLFETATLRSGYLLPDTKAYGDRIERMLRLSLNIDPEAQVEEEPEEEPEDTSEDAEDSEQDEGEEMDAGTEEEEEETEKESTEKDEL

SEQ ID NO: 19 - DNA, 20 bp, Artificial Sequence
  /note=“Description of Artificial Sequence Synthetic sgRNA targeting sequence”
gcctgctgtcacttgctacg

SEQ ID NO: 20 - DNA, 20 bp, Artificial Sequence
  /note=“Description of Artificial Sequence Synthetic sgRNA targeting sequence”
aatcaaccagagaatttccg

SEQ ID NO: 21 - DNA, 20 bp, Artificial Sequence
  /note=“Description of Artificial Sequence Synthetic sgRNA targeting sequence”
ggtccagctcccgttctaca

SEQ ID NO: 22 - DNA, 20 bp, Artificial Sequence
  /note=“Description of Artificial Sequence Synthetic sgRNA targeting sequence”
gtatggcagcaacgtcacga

SEQ ID NO: 23 - DNA, 20 bp, Artificial Sequence
  /note=“Description of Artificial Sequence Synthetic sgRNA targeting sequence”
tatagagctgaaaaaccgca

SEQ ID NO: 24 - DNA, 20 bp, Artificial Sequence
  /note=“Description of Artificial Sequence Synthetic sgRNA targeting sequence”
ccacattacggacgatgcaa

SEQ ID NO: 25 - DNA, 20 bp, Artificial Sequence
  /note=“Description of Artificial Sequence Synthetic sgRNA targeting sequence”
tcttacgaggaggagaaagg

SEQ ID NO: 26 - DNA, 20 bp, Artificial Sequence
  /note=“Description of Artificial Sequence Synthetic sgRNA targeting sequence”
gcaagtgtagtttcccacca

SEQ ID NO: 27 - DNA, 20 bp, Artificial Sequence
  /note=“Description of Artificial Sequence Synthetic sgRNA targeting sequence”
gcgaggtattcggctccgcg

SEQ ID NO: 28 - DNA, 20 bp, Artificial Sequence
  /note=“Description of Artificial Sequence Synthetic sgRNA targeting sequence”
gctttcacggaggttcgacg

SEQ ID NO: 29 - DNA, 20 bp, Artificial Sequence
  /note=“Description of Artificial Sequence Synthetic sgRNA targeting sequence”
atgttgcagttcggctcgat

SEQ ID NO: 30 - DNA, 20 bp, Artificial Sequence
  /note=“Description of Artificial Sequence Synthetic sgRNA targeting sequence”
acgtgtaaggcgaacgcctt

SEQ ID NO: 31 - DNA, 20 bp, Artificial Sequence
  /note=“Description of Artificial Sequence Synthetic sgRNA targeting sequence”
attgttcgaccgtctacggg

SEQ ID NO: 32 - DNA, 20 bp, Artificial Sequence
  /note=“Description of Artificial Sequence Synthetic sgRNA targeting sequence”
gcattgctaagaagaagcgc